SEQUENCE LISTING (43 sequences)

SEQ ID NO: 1 - protein, 332 aa, Homo sapiens
MGSRCALALAVLSALLCQVWSSGVFELKLQEFVNKKGLLGNRNCCRGGAGPPPCACRTFFRVCLKHYQASVSPEPPCTYGSAVTPVLGVDSFSLPDGGGADSAFSNPIRFPFGFTWPGTFSLIIEALHTDSPDDLATENPERLISRLATQRHLTVGEEWSQDLHSSGRTDLKYSYRFVCDEHYYGEGCSVFCRPRDDAFGHFTCGERGEKVCNPGWKGPYCTEPICLPGCDEQHGFCDKPGECKCRVGWQGRYCDECIRYPGCLHGTCQQPWQCNCQEGWGGLFCNQDLNYCTHHKPCKNGATCTNTGQGSYTCSCRPGYTGATCELGIDEC

SEQ ID NO: 2 - protein, 63 aa, Drosophila melanogaster
WKTNKSESQYTSLEYDFRVTCDLNYYGSGCAKFCRPRDDSFGHSTCSETGEIICLTGWQGDYC

SEQ ID NO: 3 - protein, 63 aa, Homo sapiens
WSQDLHSSGRTDLKYSYRFVCDEHYYGEGCSVFCRPRDDAFGHFTCGERGEKVCNPGWKGPYC

SEQ ID NO: 4 - protein, 63 aa, Mus musculus
WSQDLHSSGRTDLRYSYRFVCDEHYYGEGCSVFCRPRDDAFGHFTCGDRGEKMCDPGWKGQYC

SEQ ID NO: 5 - protein, 63 aa, Rattus sp.
WSQDLHSSGRTDLRYSYRFVCDEHYYGEGCSVFCRPRDDAFGHFTCGERGEKMCDPGWKGQYC

SEQ ID NO: 6 - protein, 63 aa, Mus musculus
WRTDEQNDTLTRLSYSYRVICSDNYYGESCSRLCKKRDDHFGHYECQPDGSLSCLPGWTGKYC

SEQ ID NO: 7 - protein, 63 aa, Homo sapiens
WLLDEQTSTLTRLRYSYRVICSDNYYGDNCSRLCKKRNDHFGHYVCQPDGNLSCLPGWTGEYC

SEQ ID NO: 8 - protein, 63 aa, Rattus sp.
WQTLKQNTGIAHFEYQIRVTCDDHYYGFGCNKFCRPRDDFFGHYACDQNGNKTCMEGWMGPEC

SEQ ID NO: 9 - protein, 63 aa, Mus musculus
WQTLKQNTGIAHFEYQIRVTCDDHYYGFGCNKFCRPRDDFFGHYACDQNGNKTCMEGWMGPDC

SEQ ID NO: 10 - protein, 63 aa, Homo sapiens
WQTLKQNTGVAHFEYQIRVTCDDYYYGFGCNKFCRPRDDFFGHYACDQNGNKTCMEGWMGREC

SEQ ID NO: 11 - protein, 63 aa, Gallus sp.
WQTLKHNTGAAHFEYQIRVTCAEHYYGFGCNKFCRPRDDFFTHHTCDQNGNKTCLEGWTGPEC

SEQ ID NO: 12 - protein, 63 aa, Gallus sp.
WKTLQFNGPVANFEVQIRVKCDENYYSALCNKFCGPRDDFVGHYTCDQNGNKACMEGWMGEEC

SEQ ID NO: 13 - protein, 63 aa, Mus musculus
WKSLHFSGHVAHLELQIRVRCDENYYSATCNKFCRPRNDFFGHYTCDQYGNKACMDGWMGKEC

SEQ ID NO: 14 - protein, 63 aa, Homo sapiens; identical to SEQ ID NO: 13

SEQ ID NO: 15 - protein, 63 aa, Rattus sp.; identical to SEQ ID NO: 13

SEQ ID NO: 16 - protein, 63 aa, Homo sapiens; identical to SEQ ID NO: 13

SEQ ID NO: 17 - protein, 63 aa, Drosophila melanogaster
WKTLDHIGRNARITYRVRVQCAVTYYNTTCTTFCRPRDDQFGHYACGSEGQKLCLNGWQGVNC

SEQ ID NO: 18 - protein, 723 aa, Homo sapiens [sequence omitted]

SEQ ID NO: 19 - protein, 618 aa, Homo sapiens
MVSPRMSGLLSQTVILALIFLPQTRPAGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYRARCEPPAVGTACTRLCRPRSAPSRCGPGLRPCAPLEDECEAPLVCRAGCSPEHGFCEQPGECRCLEGWTGPLCTVPVSTSSCLSPRGPSSATTGCLVPGPGPCDGNPCANGGSCSETPRSFECTCPRGFYGLRCEVSGVTCADGPCFNGGLCVGGADPDSAYICHCPPGFQGSNCEKRVDRCSLQPCRNGGLCLDLGHALRCRCRAGFAGPRCEHDLDDCAGRACANGGTCVEGGGAHRCSCALGFGGRDCRERADPCAARPCAHGGRCYAHFSGLVCACAPGYMGARCEFPVHPDGASALPAAPPGLRPGDPQRYLLPPALGLLVAAGVAGAALLLVHVRRRGHSQDAGSRLLAGTPEPSVHALPDALNNLRTQEGSGDGPSSSVDWNRPEDVDPQGIYVISAPSIYAREVATPLFPPLHTGRAGQRQHLLFPYPSSILSVK

SEQ ID NO: 20 - protein, 685 aa, Homo sapiens
MAAASRSASGWALLLLVALWQQRAAGSGVFQLQLQEFINERGVLASGRPCEPGCRTFFRVCLKHFQAVVSPGPCTFGTVSTPVLGTNSFAVRDDSSGGGRNPLQLPFNFTWPGTFSLIIEAWHAPGDDLRPEALPPDALISKIAIQGSLAVGQNWLLDEQTSTLTRLRYSYRVICSDNYYGDNCSRLCKKRNDHFGHYVCQPDGNLSCLPGWTGEYCQQPICLSGCHEQNGYCSKPAECLCRPGWQGRLCNECIPHNGCRHGTCSTPWQCTCDEGWGGLFCDQDLNYCTHHSPCKNGATCSNSGQRSYTCTCRPGYTGVDCELELSECDSNPCRNGGSCKDQEDGYHCLCPPGYYGLHCEHSTLSCADSPCFNGGSCRERNQGANYACECPPNFTGSNCEKKVDRCTSNPCANGGQCLNRGPSRMCRCRPGFTGTYCELHVSDCARNPCAHGGTCHDLENGLMCTCPAGFSGRRCEVRTSIDACASSPCFNRATCYTDLSTDTFVCNCPYGFVGSRCEFPVGLPPSFPWVAVSLGVGLAVLLVLLGMVAVAVRQLRLRRPDDGSREAMNNLSDFQKDNLIPAAQLKNTNQKKELEVDCGLDKSNCGKQQNHTLDYNLAPGPLGRGTMPGKFPHSDKSLGEKAPLRLHSEKPECRISAICSPRDSMYQSVCLISEERNECVIATEV

SEQ ID NO: 21 - protein, 1218 aa, Homo sapiens
MRSPRTRGRSGRPLSLLLALLCALRAKVCGASGQFELEILSMQNVNGELQNGNCCGGARNPGDRKCTRDECDTYFKVCLKEYQSRVTAGGPCSFGSGSTPVIGGNTFNLKASRGNDRNRIVLPFSFAWPRSYTLLVEAWDSSNDTVQPDSIIEKASHSGMINPSRQWQTLKQNTGVAHFEYQIRVTCDDYYYGFGCNKFCRPRDDFFGHYACDQNGNKTCMEGWMGPECNRAICRQGCSPKHGSCKLPGDCRCQYGWQGLYCDKCIPHPGCVHGICNEPWQCLCETNWGGQLCDKDLNYCGTHQPCLNGGTCSNTGPDKYQCSCPEGYSGPNCEIAEHACLSDPCHNRGSCKETSLGFECECSPGWTGPTCSTNIDDCSPNNCSHGGTCQDLVNGFKCVCPPQWTGKTCQLDANECEAKPCVNAKSCKNLIASYYCDCLPGWMGQNCDININDCLGQCQNDASCRDLVNGYRCICPPGYAGDHCERDIDECASNPCLNGGHCQNEINRFQCLCPTGFSGNLCQLDIDYCEPNPCQNGAQCYNRASDYFCKCPEDYEGKNCSHLKDHCRTTPCEVIDSCTVAMASNDTPEGVRYISSNVCGPHGKCKSQSGGKFTCDCNKGFTGTYCHENINDCESNPCRNGGTCIDGVNSYKCICSDGWEGAYCETNINDCSQNPCHNGGTCRDLVNDFYCDCKNGWKGKTCHSRDSQCDEATCNNGGTCYDEGDAFKCMCPGGWEGTTCNIARNSSCLPNPCHNGGTCVVNGESFTCVCKEGWEGPICAQNTNDCSPHPCYNSGTCVDGDNWYRCECAPGFAGPDCRININECQSSPCAFGATCVDEINGYRCVCPPGHSGAKCQEVSGRPCITMGSVIPDGAKWDDDCNTCQCLNGRIACSKVWCGPRPCLLHKGHSECPSGQSCIPILDDQCFVHPCTGVGECRSSSLQPVKTKCTSDSYYQDNCANITFTFNKEMMSPGLTTEHICSELRNLNILKNVSAEYSIYIACEPSPSANNEIHVAISAEDIRDDGNPIKEITDKIIDLVSKRDGNSSLIAAVAEVRVQRRPLKNRTDFLVPLLSSVLTVAWICCLVTAFYWCLRKRRKPGSHTHSASEDNTTNNVREQLNQIKNPIEKHGANTVPIKDYENKNSKMSKIRTHNSEVEEDDMDKHQQKARFAKQPAYTLVDREEKPPNGTPTKHPNWTNKQDNRDLESAQSLNRMEYIV

SEQ ID NO: 22 - protein, 1238 aa, Homo sapiens [sequence omitted]

SEQ ID NO: 23 - protein, 43 aa, Artificial Sequence
  Description of Artificial Sequence Synthetic consensus amino acid
CXXXXXXXXCXXXCXXXXXXXXXXXCXXXXXXXCXXXXXXXXC

SEQ ID NO: 24 - protein, 43 aa, Artificial Sequence; identical to SEQ ID NO: 23
  Description of Artificial Sequence Synthetic consensus amino acid

SEQ ID NO: 25 - protein, 43 aa, Artificial Sequence
  Description of Artificial Sequence Synthetic consensus amino acid
CXXXYYXXXCXXXCRPRBDXFGHXXCXXXGXXXCXXGWXGXXC

SEQ ID NO: 26 - protein, 175 aa, Artificial Sequence
  Description of Artificial Sequence Synthetic amino acid construct
XXXXCXXXXXXXXXXXXXXXXXXXXXXXXXXXXXXXXXXXXXXXXXXXXXXXXCXXXXXXXXXXXXCXXXXXXXXXXXXXXXXXXXXXXXXXXXXXXXXXXXXXXXXXXXXXXXXXXXXXXXXXXXXXXXXXXXXXXCXXXXXXCXXGXXXXXXXXXXXXXXXXXXXXXXGXXCX

SEQ ID NO: 27 - protein, 864 aa, Artificial Sequence
  Description of Artificial Sequence Synthetic fusion protein
MGSRCALALAVLSALLCQVWSSGVFELKLQEFVNKKGLLGNRNCCRGGAGPPPCACRTFFRVCLKHYQASVSPEPPCTYGSAVTPVLGVDSFSLPDGGGADSAFSNPIRFPFGFTWPGTFSLIIEALHTDSPDDLATENPERLISRLATQRHLTVGEEWSQDLHSSGRTDLKYSYRFVCDEHYYGEGCSVFCRPRDDAFGHFTCGERGEKVCNPGWKGPYCTEPICLPGCDEQHGFCDKPGECKCRVGWQGRYCDECIRYPGCLHGTCQQPWQCNCQEGWGGLFCNQDLNYCTHHKPCKNGATCTNTGQGSYTCSCRPGYTGATCELGIDECDPSPCKNGGSCTDLENSYSCTCPPGFYGKICELSAMTCADGPCFNGGRCSDSPDGGYSCRCPVGYSGFNCEKKIDYCSSSPCSNGAKCVDLGDAYLCRCQAGFSGRHCDDNVDDCASSPCANGGTCRDGVNDFSCTCPPGYTGRNCSAPVSRCEHAPCHNGATCHERGHGYVCECARGYGGPNCQFLLPELPPGPAVVDLTEKLEASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 28 - DNA, 20 bp, Artificial Sequence
  Description of Artificial Sequence Synthetic primer
gtaacccgttgaaccccatt

SEQ ID NO: 29 - DNA, 20 bp, Artificial Sequence
  Description of Artificial Sequence Synthetic primer
ccatccaatcggtagtagcg

SEQ ID NO: 30 - DNA, 20 bp, Artificial Sequence
  Description of Artificial Sequence Synthetic primer
ggtgctgataacagcggaat

SEQ ID NO: 31 - DNA, 20 bp, Artificial Sequence
  Description of Artificial Sequence Synthetic primer
atttttggaatccttcacgc

SEQ ID NO: 32 - DNA, 20 bp, Artificial Sequence
  Description of Artificial Sequence Synthetic primer
gtaacccgttgaaccccatt

SEQ ID NO: 33 - DNA, 20 bp, Artificial Sequence
  Description of Artificial Sequence Synthetic primer
ccatccaatcggtagtagcg

SEQ ID NO: 34 - DNA, 20 bp, Artificial Sequence
  Description of Artificial Sequence Synthetic primer
ccagggagatcctttgatga

SEQ ID NO: 35 - DNA, 20 bp, Artificial Sequence
  Description of Artificial Sequence Synthetic primer
cattcccagaggaattgcat

SEQ ID NO: 36 - DNA, 20 bp, Artificial Sequence
  Description of Artificial Sequence Synthetic primer
gtgattgcggggttgtatct

SEQ ID NO: 37 - DNA, 20 bp, Artificial Sequence
  Description of Artificial Sequence Synthetic primer
acatctcctcccatcagcag

SEQ ID NO: 38 - DNA, 20 bp, Artificial Sequence
  Description of Artificial Sequence Synthetic primer
gatcgtcctgcagtctctcc

SEQ ID NO: 39 - DNA, 20 bp, Artificial Sequence
  Description of Artificial Sequence Synthetic primer
agttctcccggaatcctttg

SEQ ID NO: 40 - DNA, 20 bp, Artificial Sequence
  Description of Artificial Sequence Synthetic primer
agagggtgcagcagagacac

SEQ ID NO: 41 - DNA, 20 bp, Artificial Sequence
  Description of Artificial Sequence Synthetic primer
catgaaaaattcgggagagg

SEQ ID NO: 42 - DNA, 20 bp, Artificial Sequence
  Description of Artificial Sequence Synthetic primer
ctggaggaggaacgctaatg

SEQ ID NO: 43 - DNA, 17 bp, Artificial Sequence
  Description of Artificial Sequence Synthetic primer
gttgaaggagctcttgg

(19) United States
(12) Patent Application Publication
(10) Pub. No.: US 2006/0128619 A1
(43) Pub. Date: Jun. 15, 2006

(54) Therapeutic use of modulators of notch

(76) Inventors: Brian Robert Champion, Cambridge (GB); Lesley Lynn Young, Cambridge (GB); Grahame James McKenzie, Cambridge (GB)

Correspondence Address: FROMMER LAWRENCE & HAUG, 745 FIFTH AVENUE- 10TH FL., NEW YORK, NY, 10151, US

(21) Appl. No.: 11/178,724

(22) Filed: Jul. 11, 2005

Related U.S. Application Data
(63) Continuation-in-part of application No. PCT/GB04/00021, filed on Jan. 9, 2004.

(30) Foreign Application Priority Data
Jan. 9, 2003 (GB) 0300428.0

(51) Int. Cl. A61K 48/00; A61K 38/17; C07K 14/705

(52) U.S. Cl. 514/12; 514/44; 530/350

(57) ABSTRACT

Provided is a method for modifying IL-4 expression in a cell using a modulator of Notch signalling. Also provided are methods for generating immune modulatory cytokine profiles with increased IL-4 expression and/or increased IL-10 expression and/or reduced IL-5, IL-13 and TNFα expression. In addition, a method for increasing a TH2 immune response and/or decreasing a TH1 immune response in a cell, using a modulator of Notch signalling, is provided. Methods of treatment are also disclosed.

### CROSS REFERENCE TO RELATED APPLICATIONS

This application is a continuation-in-part of International Application No. PCT/GB2004/000021, filed on Jan. 9, 2004, published as WO 2004/062686 on Jul. 29, 2004, and claiming priority to GB Application Serial No. 0300428.0, filed Jan. 9, 2003.

Reference is made to U.S. application Ser. No. 09/310,685, filed May 4, 1999; Ser. No. 09/870,902, filed May 31, 2001; Ser. No. 10/013,310, filed Dec. 7, 2001; Ser. No. 10/147,354, filed May 16, 2002; Ser. No. 10/357,321, filed Feb. 3, 2002; Ser. No. 10/682,230, filed Oct. 9, 2003; Ser. No. 10/720,896, filed Nov. 24, 2003; Ser. Nos. 10/763,362, 10/764,415 and 10/765,727, all filed Jan. 23, 2004; Ser. No. 10/812,144, filed Mar. 29, 2004; Ser. No. 10/845,834 and Ser. No. 10/846,989, both filed May 14, 2004; Ser. No. 10/877,563, filed Jun. 25, 2004; Ser. No. 10/899,422, filed Jul. 26, 2004; Ser. No. 10/958,784, filed Oct. 5, 2004; Ser. No. 11/050,328, filed Feb. 3, 2005; Ser. No. 11/058,066, filed Feb. 14, 2005; Ser. No. 11/071,796, filed Mar. 3, 2005; Ser. No. 11/078,735, filed Mar. 10, 2005 and Ser. No. 11/103,077, filed Apr. 11, 2005.

All of the foregoing applications, as well as all documents cited in the foregoing applications (“application documents”) and all documents cited or referenced in the application documents are incorporated herein by reference. Also, all documents cited in this application (“herein-cited documents”) and all documents cited or referenced in herein-cited documents are incorporated herein by reference. In addition, any manufacturer's instructions or catalogues for any products cited or mentioned in each of the application documents or herein-cited documents are incorporated by reference. Documents incorporated by reference into this text or any teachings therein can be used in the practice of this invention. Documents incorporated by reference into this text are not admitted to be prior art.

### FIELD OF THE INVENTION

The present invention relates inter alia to uses of modulators of Notch signalling in therapy and corresponding methods of treatment, in particular to modulate IL-4 expression and control Th1, Th2 and/or Treg responses.

### BACKGROUND OF THE INVENTION

Notch signal transduction plays a critical role in cell fate determination in vertebrate and invertebrate tissues. Notch is expressed at many stages of Drosophila embryonic and larval development and in many different cells implying a wide range of functions including an important role in neurogenesis and in the differentiation of mesodermal and endodermal cells. There are at least four mammalian Notch genes (Notch-1, Notch-2, Notch-3 and Notch-4). Notch-1, which most vertebrates, is widely expressed and is essential for early development. Recent evidence suggests that Notch signalling contributes to lineage commitment of immature T-cells in the thymus.

During maturation in the thymus, T-cells acquire the ability to distinguish self-antigens from those that are non-self, a process termed “self tolerance”. Tolerance to a non-self antigen, however, may be induced by immunisation under specific conditions with a peptide fragment comprising that antigen. In autoimmune diseases such as multiple sclerosis, rheumatoid arthritis or diabetes, there is a failure of the proper regulation of tolerance. Improved treatment methods for re-establishing tolerance are desirable for autoimmune diseases. Similarly in allergic conditions and for transplantation of an organ or tissue from a donor individual, induction of tolerance to particular foreign antigens or profiles of foreign antigens is desirable.

The expression on the cell surface of normal adult cells of the peripheral immune system of Notch and its ligands, Delta and Serrate, suggests a role for these proteins in T-cell acquired immunocompetence. T-cells express Notch-1 mRNA constitutively. Delta expression is limited to only a subset of T-cells in the peripheral lymphoid tissues. Serrate expression is restricted to a subset of antigen presenting cells (APCs). These observations reinforce the view that the Notch receptor ligand family continues to regulate cell fate decisions in the immune system beyond embryonic development with Notch signalling playing a central role in the induction of peripheral unresponsiveness (tolerance or anergy), linked suppression and infectious tolerance (Hoyne et al.).

Thus, as described in WO 98/20142, manipulation of the Notch signalling pathway can be used in immunotherapy and in the prevention and/or treatment of T-cell mediated diseases. In particular, allergy, autoimmunity, graft rejection, tumour induced aberrations to the T-cell system and infectious diseases caused, for example, by Plasmodium species, Microfilariae, Helminths, Mycobacteria, HIV, Cytomegalovirus, Pseudomonas, Toxoplasma, Echinococcus, Haemophilus influenza type B, measles, Hepatitis C or Toxicara, may be targeted.

It has also recently been shown that it is possible to generate a class of regulatory T cells which are able to transmit antigen-specific tolerance to other T cells, a process termed infectious tolerance (WO98/20142). The functional activity of these cells can be mimicked by over-expression of a Notch ligand protein on their cell surfaces or on the surface of antigen presenting cells. In particular, regulatory T cells can be generated by over-expression of a member of the Delta or Serrate family of Notch ligand proteins. Delta or Serrate induced T cells specific to one antigenic epitope are also able to transfer tolerance to T cells recognising other epitopes on the same or related antigens, a phenomenon termed “epitope spreading”.

Notch ligand expression also plays a role in cancer. Indeed, upregulated Notch ligand expression has been observed in some tumour cells. These tumour cells are capable of rendering T cells unresponsive to restimulation with a specific antigen, thus providing a possible explanation of how tumour cells prevent normal T cell responses. By downregulating Notch signalling in vivo in T cells, it may be possible to prevent tumour cells from inducing immunotolerance in those T cells that recognise tumour-specific antigens. In turn, this would allow the T cells to mount an immune response against the tumour cells (WO00/135990).

A description of the Notch signalling pathway and conditions affected by it may be found, for example, in our published PCT Applications as follows:

PCT/GB97/03058 (filed on 6 Nov. 1997 and published as WO 98/20142; claiming priority from GB 9623236.8 filed on 7 Nov. 1996, GB 9715674.9 filed on 24 Jul. 1997 and GB 9719350.2 filed on 11 Sep. 1997);

PCT/GB99/04233 (filed on 15 Dec. 1999 and published as WO 00/36089; claiming priority from GB 9827604.1 filed on 15 Dec. 1999);

PCT/GB00/04391 (filed on 17 Nov. 2000 and published as WO 0135990; claiming priority from GB 9927328.6 filed on 18 Nov. 1999);

PCT/GB01/03503 (filed on 3 Aug. 2001 and published as WO 02/12890; claiming priority from GB 0019242.7 filed on 4 Aug. 2000);

PCT/GB02/02438 (filed on 24 May 2002 and published as WO 02/096952; claiming priority from GB 0112818.0 filed on 25 May 2001);

PCT/GB02/03381 (filed on 25 Jul. 2002 and published as WO 03/012111; claiming priority from GB 0118155.1 filed on 25 Jul. 2001);

PCT/GB02/03397 (filed on 25 Jul. 2002 and published as WO 03/012441; claiming priority from GB0118153.6 filed on 25 Jul. 2001, GB0207930.9 filed on 5 Apr. 2002, GB 0212282.8 filed on 28 May 2002 and GB 0212283.6 filed on 28 May 2002);

PCT/GB02/03426 (filed on 25 Jul. 2002 and published as WO 03/011317; claiming priority from GB0118153.6 filed on 25 Jul. 2001, GB0207930.9 filed on 5 Apr. 2002, GB 0212282.8 filed on 28 May 2002 and GB 0212283.6 filed on 28 May 2002);

PCT/GB02/04390 (filed on 27 Sep. 2002 and published as WO 03/029293; claiming priority from GB 0123379.0 filed on 28 Sep. 2001);

PCT/GB02/05137 (filed on 13 Nov. 2002 and published as WO 03/041735; claiming priority from GB 0127267.3 filed on 14 Nov. 2001, PCT/GB02/03426 filed on 25 Jul. 2002, GB 0220849.4 filed on 7 Sep. 2002, GB 0220913.8 filed on 10 Sep. 2002 and PCT/GB02/004390 filed on 27 Sep. 2002);

PCT/GB02/05133 (filed on 13 Nov. 2002 and published as WO 03/042246; claiming priority from GB 0127271.5 filed on 14 Nov. 2001 and GB 0220913.8 filed on 10 Sep. 2002).

Each of PCT/GB97/03058 (WO 98/20142), PCT/GB99/04233 (WO 00/36089), PCT/GB00/04391 (WO 0135990), PCT/GB01/03503 (WO 02/12890), PCT/GB02/02438 (WO 02/096952), PCT/GB02/03381 (WO 03/012111), PCT/GB02/03397 (WO 03/012441), PCT/GB02/03426 (WO 03/011317), PCT/GB02/04390 (WO 03/029293), PCT/GB02/05137 (WO 03/041735) and PCT/GB02/05133 (WO 03/042246) is hereby incorporated herein by reference.

Reference is made also to Hoyne G. F. et al. (1999) Int Arch Allergy Immunol 118:122-124; Hoyne et al. (2000) Immunology 100:281-288; Hoyne G. F. et al. (2000) Intl Immunol 12:177-185; Hoyne, G. et al. (2001) Imunological Reviews 182:215-227; each of which is also incorporated herein by reference.

The present invention seeks to provide further methods of modulating the immune system.

### SUMMARY OF THE INVENTION

According to a first aspect of the invention there is provided a method for modifying IL-4 expression by administering a modulator of Notch signalling.

According to a further aspect of the invention there is provided method for increasing IL-4 expression by administering an activator of Notch signalling.

The invention further provides a method for reducing IL-4 expression by administering an inhibitor of Notch signalling.

The invention further provides a method for increasing IL-4 expression by administering a Notch receptor agonist or partial agonist.

The invention further provides a method for reducing IL-4 expression by administering a Notch receptor antagonist.

Suitably the modulator of Notch signalling modifies cytokine (IL-4 etc) expression in leukocytes, fibroblasts or epithelial cells.

Suitably the modulator of Notch signalling modifies cytokine expression in lymphocytes or macrophages.

Whilst not wishing to be bound by any theory or mechanism, it is suggested that activators of Notch signalling are capable of modifying the TH1/TH2 balance of an immune response in favour of a TH2 response and away from a TH1 response. This may be achieved, for example, by directing T cell development through a TH2/Treg precursor cell type. This is considered to be useful, for example, for the treatment of TH1 mediated disorders such as autoimmune diseases (e.g. organ-specific autoimmune diseases) and graft rejection.

Thus, the invention further provides a method for modifying the TH1/TH2 balance of an immune response away from a TH1 response and/or towards a TH2 response by administering a modulator of Notch signalling.

In a preferred form, the invention provides a method for modifying the TH1/TH2 balance of an immune response away from a TH1 response and/or towards a TH2 response by administering an activator of a Notch receptor.

The invention further provides a method for modifying the TH1/TH2 balance of an immune response in favour of a TH2 response by administering a modulator of Notch signalling.

The invention further provides a method for modifying the TH1/TH2 balance of an immune response in favour of a TH2 response by administering an activator of a Notch receptor.

Suitably the method is used to treat a TH1 mediated disease such as graft rejection or autoimmune disease (e.g. organ-specific autoimmune disease).

Suitably the autoimmune disorder is selected from the group consisting of multiple sclerosis, insulin-dependent diabetes, sympathetic ophthalmia, uveitis and psoriasis.

According to a further aspect of the invention there is provided a method for generating an immune modulatory cytokine profile with increased IL-10 expression and increased IL-4 expression by administering a modulator of Notch signalling.

According to a further aspect of the invention there is provided a method for generating an immune modulatory cytokine profile with increased IL-4 expression and reduced IL-5, IL-13 and TNFα expression by administering a modulator of Notch signalling.

According to a further aspect of the invention there is provided a method for generating an immune modulatory cytokine profile with increased IL-4 expression and reduced IL-2, IFNγ, IL-5, IL-13 and TNFα expression by administering a modulator of Notch signalling.

Suitably the cytokine profile also exhibits increased IL-10 expression.

According to a further aspect of the invention there is provided a method for increasing a TH2 immune response by administering a modulator of Notch signalling to increase IL-4 expression.

According to a further aspect of the invention there is provided a method for reducing a TH1 immune response by administering a modulator of Notch signalling to increase IL-4 expression.

According to a further aspect of the invention there is provided a method for increasing a TH2 immune response and reducing a TH1 immune response by administering a modulator of Notch signalling to increase IL-4 expression.

According to a further aspect of the invention there is provided a method for treating inflammation or an inflammatory condition by administering a modulator of Notch signalling to increase IL-4 expression and reduce a TH1 immune response.

According to a further aspect of the invention there is provided a method for treating inflammation or an inflammatory or autoimmune condition by administering a modulator of Notch signalling to increase IL-4 expression and reduce a TH1 immune response.

Suitably the modulator of Notch signalling is administered to a patient in vivo.

Alternatively the modulator of Notch signalling is administered to a cell ex-vivo, after which the cell may be administered to a patient.

Suitably the method may be used to treat a disorder selected from the group consisting of: thyroiditis, insulitis, multiple sclerosis, iridocyclitis, uveitis, orchitis, hepatitis, Addison's disease, myasthenia gravis, rheumatoid arthritis, lupus erythematosus, immune hyperreactivity, insulin dependent diabetes mellitus, anemia (aplastic, hemolytic), autoimmune hepatitis, skleritis, idiopathic thrombocytopenic purpura, inflammatory bowel diseases (Crohn's disease, ulcerative colitis), juvenile arthritis, scleroderma and systemic sclerosis, sjogren's syndrom, undifferentiated connective tissue syndrome, antiphospholipid syndrome, vasculitis (polyarteritis nodosa, allergic granulomatosis and angiitis, Wegner's granulomatosis, Kawasaki disease, hypersensitivity vasculitis, Henoch-Schoenlein purpura, Behcet's Syndrome, Takayasu arteritis, Giant cell arteritis, Thrombangiitis obliterans), polymyalgia rheumatica, essentiell (mixed) cryoglobulinemia, Psoriasis vulgaris and psoriatic arthritis, diffus fasciitis with or without eosinophilia, polymyositis and other idiopathic inflammatory myopathies, relapsing panniculitis, relapsing polychondritis, lymphomatoid granulomatosis, erythema nodosum, ankylosing spondylitis, Reiter's syndrome, inflammatory dermatitis, unwanted immune reactions and inflammation associated with arthritis, including rheumatoid arthritis, inflammation associated with hypersensitivity and allergic reactions, systemic lupus erythematosus, collagen diseases, inflammation associated with atherosclerosis, arteriosclerosis, atherosclerotic heart disease, reperfusion injury, cardiac arrest, myocardial infarction, vascular inflammatory disorders, respiratory distress syndrome or other cardiopulmonary diseases, inflammation associated with peptic ulcer, ulcerative colitis and other diseases of the gastrointestinal tract, hepatic fibrosis, liver cirrhosis or other hepatic diseases, thyroiditis or other glandular diseases, glomerulonephritis or other renal and urologic diseases, otitis or other oto-rhino-laryngological diseases, dermatitis or other dermal diseases, periodontal diseases or other dental diseases, orchitis or epididimo-orchitis, infertility, orchidal trauma or other immune-related testicular diseases, placental dysfunction, placental insufficiency, habitual abortion, eclampsia, pre-eclampsia and other immune and/or inflammatory-related gynaecological diseases, posterior uveitis, intermediate uveitis, anterior uveitis, conjunctivitis, chorioretinitis, uveoretinitis, optic neuritis, intraocular inflammation, e.g. retinitis or cystoid macular oedema, sympathetic ophthalmia, scleritis, retinitis pigmentosa, immune and inflammatory components of degenerative fondus disease, inflammatory components of ocular trauma, ocular inflammation caused by infection, proliferative vitreo-retinopathies, acute ischaemic optic neuropathy, excessive scarring, e.g. following glaucoma filtration operation, immune and/or inflammation reaction against ocular implants and other immune and inflammatory-related ophthalmic diseases, inflammation associated with autoimmune diseases or conditions or disorders where, both in the central nervous system (CNS) or in any other organ, immune and/or inflammation suppression would be beneficial, Parkinson's disease, complication and/or side effects from treatment of Parkinson's disease, AIDS-related dementia complex HIV-related encephalopathy, Devic's disease, Sydenham chorea, Alzheimer's disease and other degenerative diseases, conditions or disorders of the CNS, inflammatory components of strokes, post-polio syndrome, immune and inflammatory components of psychiatric disorders, myelitis, encephalitis, subacute sclerosing pan-encephalitis, encephalomyelitis, acute neuropathy, subacute neuropathy, chronic neuropathy, Guillaim-Barre syndrome, Sydenham chora, pseudo-tumour cerebri, Down's Syndrome, Huntington's disease, amyotrophic lateral sclerosis, inflammatory components of CNS compression or CNS trauma or infections of the CNS, inflammatory components of muscular atrophies and dystrophies, and immune and inflammatory related diseases, conditions or disorders of the central and peripheral nervous systems, post-traumatic inflammation, septic shock, infectious diseases, inflammatory complications or side effects of surgery or organ, inflammatory and/or immune complications and side effects of gene therapy, e.g. due to infection with a viral carrier, or inflammation associated with AIDS, to suppress or inhibit a humoral and/or cellular immune response, to treat or ameliorate monocyte or leukocyte proliferative diseases, e.g. leukaemia, by reducing the amount of monocytes or lymphocytes, for the prevention and/or treatment of graft rejection in cases of transplantation of natural or artificial cells, tissue and organs such as cornea, bone marrow, organs, lenses, pacemakers, natural or artificial skin tissue.

According to a further aspect of the invention there is provided the use of a modulator of Notch signalling to modify IL-4 expression.

According to a further aspect of the invention there is provided the use of an activator of Notch signalling to increase IL-4 expression.

According to a further aspect of the invention there is provided the use of an inhibitor of Notch signalling to reduce IL-4 expression.

According to a further aspect of the invention there is provided the use of a Notch receptor agonist to increase IL-4 expression.

According to a further aspect of the invention there is provided the use of a Notch receptor antagonist to reduce IL-4 expression.

According to a further aspect of the invention there is provided the use of a modulator of Notch signalling for modifying the TH1/TH2 balance of an immune response away from a TH1 response.

According to a further aspect of the invention there is provided the use of a modulator of Notch signalling for modifying the TH 1/TH2 balance of an immune response away from a TH1 response.

According to a further aspect of the invention there is provided the use of a modulator of Notch signalling for modifying the TH1/TH2 balance of an immune response in favour of a TH2 response.

According to a further aspect of the invention there is provided the use of an activator of Notch signalling for modifying the TH1/TH2 balance of an immune response in favour of a TH2 response.

According to a further aspect of the invention there is provided the use of a modulator of Notch signalling to generate an immune modulatory cytokine profile with increased IL-10 expression and increased IL-4 expression.

According to a further aspect of the invention there is provided the use of a modulator of Notch signalling to generate an immune modulatory cytokine profile with increased IL-4 expression and reduced IL-5, IL-13 and TNFα expression. Suitably the cytokine profile also exhibits increased IL-10 expression.

According to a further aspect of the invention there is provided the use of a modulator of Notch signalling to generate an immune modulatory cytokine profile with increased IL-4 expression and reduced IL-2, IFNγ, IL-5, IL-13 and TNFα expression. Suitably the cytokine profile also exhibits increased IL-10 expression.

According to a further aspect of the invention there is provided the use of a modulator of Notch signalling for increasing a TH2 immune response.

According to a further aspect of the invention there is provided the use of a modulator of Notch signalling for reducing a TH1 immune response.

According to a further aspect of the invention there is provided the use of a modulator of Notch signalling for increasing a TH2 immune response and reducing a TH1 immune response.

According to a further aspect of the invention there is provided the use of a modulator of Notch signalling for treating inflammation or an inflammatory condition by increasing IL-4 expression and reducing a TH1 immune response.

According to a further aspect of the invention there is provided the use of a modulator of Notch signalling for treating inflammation or an inflammatory or autoimmune condition by increasing IL-4 expression and reducing a TH1 immune response.

According to a further aspect of the invention there is provided the use of a modulator of Notch signalling to treat a disease associated with excessive IL-4 production.

Suitably the modulator of Notch signalling comprises a protein or polypeptide comprising a Notch ligand DSL domain or a polynucleotide sequence coding for such a protein or polypeptide.

Suitably the modulator of Notch signalling comprises a protein or polypeptide comprising a Notch ligand DSL domain and an EGF-like domain or a polynucleotide sequence coding for such a protein or polypeptide.

Suitably in such proteins and polypeptides the DSL or EGF domains are from Delta (e.g. human Delta1, Delta3 or Delta4) or Serrate/Jagged (e.g. human Jagged1 or Jagged2).

Suitably the modulator of the Notch signalling pathway comprises a fusion protein comprising a segment of a Notch ligand extracellular domain and an immunoglobulin F, segment or a polynucleotide coding for such a fusion protein.

Suitably the modulator of the Notch signalling pathway comprises a fusion protein comprising a segment of a Notch ligand extracellular domain and an immunoglobulin F, segment (e.g. IgG1 Fc or IgG4 Fc) or a polynucleotide coding for such a fusion protein. Such fusion proteins are described, for example in Example 2 of WO 98/20142. IgG fusion proteins may be prepared as well known in the art, for example, as described in U.S. Pat. No. 5,428,130 (Genentech).

Suitably a modulator of Notch signalling for use in the present invention may comprise a protein or polypeptide comprising:

i) a Notch ligand DSL domain;

ii) 1-5 (and suitably not more than 5) Notch ligand EGF domains;

iii) optionally all or part of a Notch ligand N-terminal domain; and

iv) optionally one or more heterologous amino acid sequences;

or a polynucleotide coding therefor.

Suitably a modulator of Notch signalling may comprise a protein or polypeptide comprising:

i) a Notch ligand DSL domain;

ii) 2-4 (and suitably not more than 4) Notch ligand EGF domains;

iii) optionally all or part of a Notch ligand N-terminal domain; and

iv) optionally one or more heterologous amino acid sequences;

or a polynucleotide coding therefor.

Suitably a modulator of Notch signalling may comprise a protein or polypeptide comprising:

i) a Notch ligand DSL domain;

ii) 2-3 (and suitably not more than 3) Notch ligand EGF domains;

iii) optionally all or part of a Notch ligand N-terminal domain; and

iv) optionally one or more heterologous amino acid sequences;

or a polynucleotide coding therefor.

Suitably the protein or polypeptide may have at least 50%, preferably at least 70%, preferably at least 90%, for example at least 95% amino acid sequence similarity (or preferably sequence identity) to the following sequence along the entire length of the latter (SEQ ID NO:1):

MGSRCALALAVLSALLCQVWSSGVFELKLQEFVNKKGLLGNRNCCRGGAG
PPPCACRTFFRVCLKHYQASVSPEPPCTYGSAVTPVLGVDSFSLPDGGGA
DSAFSNPIRFPFGFTWPGTFSLIIEALHTDSPDDLATENPERLISRLATQ
RHLTVGEEWSQDLHSSGRTDLKYSYRFVCDEHYYGEGCSVFCRPRDDAFG
HFTCGERGEKVCNPGWKGPYCTEPICLPGCDEQHGFCDKPGECKCRVGWQ
GRYCDECIRYPGCLHGTCQQPWQCNCQEGWGGLFCNQDLNYCTHHKPCKN
GATCTNTGQGSYTCSCRPGYTGATCELGIDEC

According to a further aspect of the invention there is provided a method for detecting, measuring or monitoring Notch signalling comprising determining the amount of a Th1- or Th2-specific transcription factor protein, polypeptide or polynucleotide or determining the amount of a polynucleotide coding for such a protein or polypeptide.

Typically, the amount of the Th1- or Th2-specific transcription factor protein, polypeptide or polynucleotide in a biological sample taken from a subject is determined, in which case the sample suitably comprises blood, serum, urine, lymphatic fluid, or tissue, suitably an immune cell, cancer cell or stem cell.

In one preferred embodiment the sample comprises peripheral T-cells.

Suitably the method comprises the steps of:

i) obtaining a biological sample from a subject; and

ii) contacting the biological sample with a binding agent that binds to a Th1- or Th2-specific transcription factor, polypeptide or polynucleotide.

Suitably the method comprises the steps of:

i) obtaining a biological sample from a subject;

ii) contacting the biological sample with a binding agent that binds to a Th1- or Th2-specific transcription factor protein, polypeptide or polynucleotide; and

iii) detecting in the sample an amount of Th1- or Th2-specific transcription factor protein, polypeptide or polynucleotide that binds to the binding agent.

Suitably the method comprises the steps of:

i) obtaining a biological sample from the patient;

ii) contacting the biological sample with a binding agent that binds to a Th1- or Th2-specific transcription factor protein, polypeptide or polynucleotide;

iii) detecting in the sample an amount of Th1- or Th2-specific transcription factor protein, polypeptide or polynucleotide that binds to the binding agent; and

iv) comparing the amount of Th1- or Th2-specific transcription factor protein, polypeptide or polynucleotide to a reference value and therefrom determining the degree of Notch signalling.

Suitably the binding agent is a protein or polypeptide, for example an antibody or antibody fragment, suitably such an antibody or antibody fragment will be specific for a human Th1- or Th2-specific transcription factor.

In an alternative embdoiment, the binding agent may be a polynucleotide, in which case the method will suitably comprise the further step of amplifying a Th1- or Th2-specific transcription factor polynucleotide in a sample and detecting the amplified polynucleotide.

Suitably such amplification may be by PCR, e.g. real-time PCR.

Suitably the Th1-specific transcription factor may be ‘T-box expressed in T cells,’

(Thet). A decrease in expression the Th1-specific transcription factor indicates a reduction in a Th1 response and vice versa.

Thet is a member of the T-box family of transcription factors that appears to regulate lineage commitment in CD4 T helper cells in part by activating the hallmark Th1 cytokine, IFN-gamma (see e.g. Zhang W X, Yang S Y, Genomics. 2000 Nov. 15;70(1):41-8). As reported by Zhang, the T-box is a strongly conserved protein domain, 174 to 186 amino acids in length that binds DNA.

Suitably the Th2-specific transcription factor protein may be cMaf or GATA3. An increase in expression the Th1-specific transcription factor indicates an increase in a Th2 response and vice versa.

According to a further aspect of the invention there is provided a method of detecting, measuring or monitoring Notch signalling in an immune cell by determining the amount of a Th1- or Th2-specific transcription factor protein, polypeptide or polynucleotide or a polynucleotide coding for such a protein or polypeptide in the cell.

Suitably the immune cell may be a T-cell, B-cell, or antigen presenting cell (APC).

According to a further aspect of the invention there is provided a method for detecting, measuring or monitoring immunological tolerance or activity comprising determining the amount of a Th1- or Th2-specific transcription factor protein, polypeptide or polynucleotide or a polynucleotide coding for such a protein or polypeptide.

According to a further aspect of the invention there is provided a method for detecting, measuring or monitoring the reactivity of a T-cell to an antigen comprising determining the amount of a Th1- or Th2-specific transcription factor protein, polypeptide or polynucleotide or a polynucleotide coding for such a protein or polypeptide.

Suitably the method will comprise a step of comparing the amount of a Th1- or Th2-specific transcription factor protein, polypeptide or polynucleotide or a polynucleotide coding for such a protein or polypeptide with a reference amount.

Suitably the amount of Th1- or Th2-specific transcription factor protein, polypeptide or polynucleotide or a polynucleotide coding for such a protein or polypeptide may be detected using a nucleic acid assay.

Alternatively, the amount of Th1- or Th2-specific transcription factor protein, polypeptide or polynucleotide or a polynucleotide coding for such a protein or polypeptide may be detected using a protein assay.

According to a further aspect of the invention there is provided a diagnostic kit comprising a binding agent that binds to a Th1- or Th2-specific transcription factor protein, polypeptide or polynucleotide for detecting, measuring or monitoring Notch signalling.

### BRIEF DESCRIPTION OF THE DRAWINGS

Various preferred features and embodiments of the present invention will now be described by way of non-limiting examples and with reference to the accompanying drawings in which:

FIG. 1 shows a schematic representation of the Notch signalling pathway;

FIG. 2 shows schematic representations of the Notch ligands Jagged and Delta;

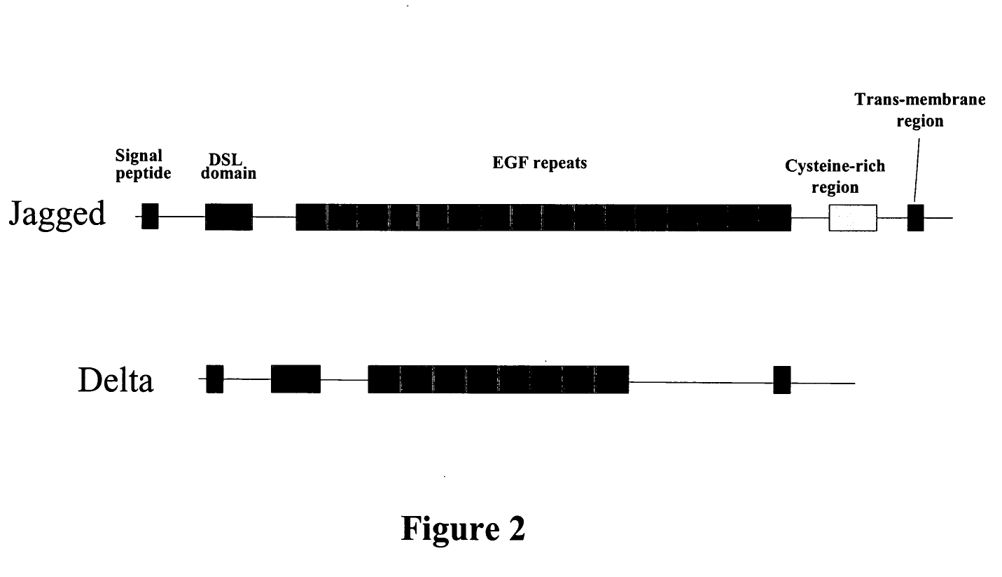

FIG. 3 shows aligned amino acid sequences (SEQ ID NO:2-17) of DSL domains from various Drosophila and mammalian Notch ligands;

FIG. 4 shows amino acid sequences of human Delta-1 (FIG. 4A; SEQ ID NO:18), human Delta-3 (FIG. 4B; SEQ ID NO:19) and human Delta-4 (FIG. 4C; SEQ ID NO:20);

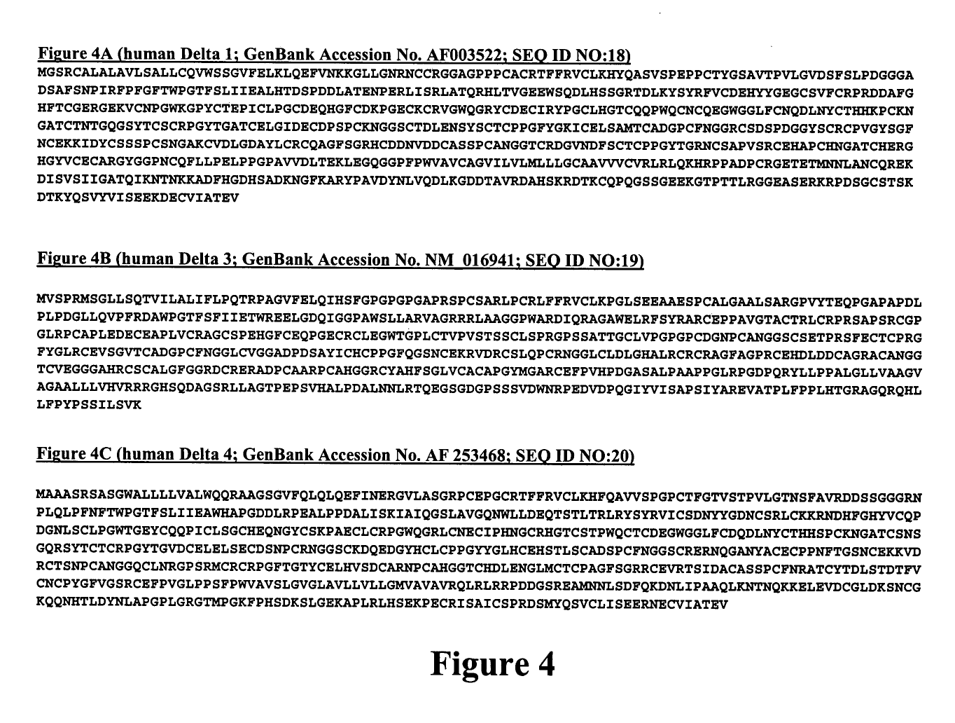

FIG. 5 shows amino acid sequences of human Jagged-1 (FIG. 5A; SEQ ID NO:21) and human Jagged-2 (FIG. 5B; SEQ ID NO:22);

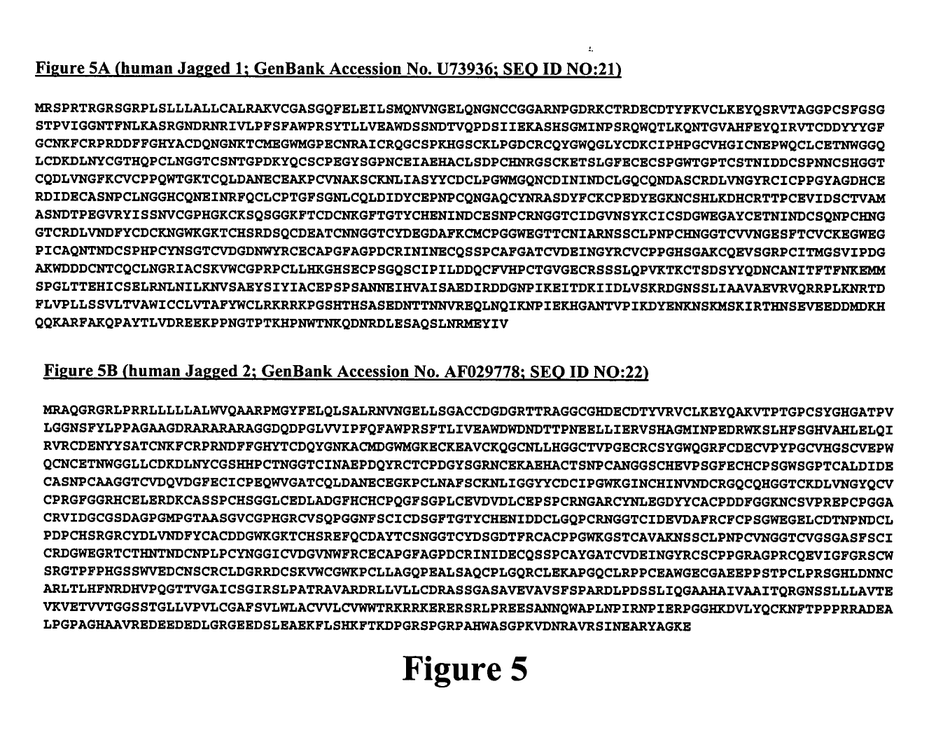

FIG. 6 shows schematic representations of modulators of Notch signalling which may be used in the present invention;

FIG. 7 shows primary polyclonal stimulation of CD4⁺ cells (results of Example 3);

FIG. 8 shows the relative expression of mHes1 in CD4⁺ cells (results of Example 4);

FIG. 9 shows cytokine production in CD4⁺ cells under polarising conditions (results of Example 5);

FIG. 10 shows cytokine concentration in control cultures and those with soluble or plate-bound Fc-delta (results of Example 6);

FIG. 11 shows cytokine concentration under various conditions (results of Example 7);

FIG. 12 shows stable CHO-Notch2-10xCBF1-Luc reporter cell line described in Example 8 with (A) plate-immobilised human Delta-1/Ig4Fc fusion protein, (B) plate-immobilised mouse Delta-1/Ig4Fc fusion protein, (C)CHO/CHO-human Delta1 co-cultured cells and (D) A20/A20-mouse Delta1 co-cultured cells as actives against corresponding controls;

FIG. 13 shows CD4+ cells activated with Delta-Fc coated beads plus soluble anti-CD28, 3d; Delta-Fc coated beads modulate in vitro T-cell responses (results of Example 10);

FIGS. 14A and 14B show the increase in IL-10 production in the presence of mouse or human Delta1 beads (results of Example 11);

FIGS. 15A and 15B show the decrease in IL-5 production in the presence of mouse or human Delta1 beads (results of Example 11);

FIGS. 16A and 16B show the increase in IL-10 production and decrease in IL-5 production in the presence of mouse Delta1 beads (results of Example 11);

FIG. 17 shows cytokine levels in human CD4+ cellS stimulated with anti-CD3 and anti-CD28, with or without mouse Delta1-hIgG4-coated beads; mDelta1-Fc enhances IL-10 production and decreases IFNγ, IL-5 and TNFα production by human CD4+ T-cells (results of Example 11);

FIG. 18 shows that IL-10 production by human CD4+ cells stimulated with anti-CD3/CD28 is enhanced by Delta1; bar graphs show varying Delta coated bead:cell ratios (results of Example 12);

FIG. 19 shows a comparison of IL-10 and IL-5 production between CD4+ naïve cells and memory cells (results of Example 13);

FIG. 20 shows cytokine production in stimulated mouse CD4+ cells under polarising conditions (results of Example 14);

FIG. 21 shows the effect of Notch activation by Delta protein on IL-4 expression in CD4+ cells (results of Example 15);

FIG. 22 shows that Delta4 (10 mg/ml) enhances IL-4 and IL-10, but decreases IL-13 secretion by anti-CD3/28 activated mouse T-cells (results of Example 16);

FIG. 23 shows cytokine regulation by Delta1 protein in anti-CD3/28 activated mouse T cells (results of Example 17); and

FIG. 24 shows transcription factor expression by T cells activated under neutral, Th1 or Th2 culture conditions with or without Delta1 (results of Example 18).

### DETAILED DESCRIPTION

The practice of the present invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Pr supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In situ Hybridization: Principles and Practice; Oxford University Press; M. J. Gait (Editor), 1984, Oligonucleotide Synthesis: A Practical Approach, Irl Press; and, D. M. J. Lilley and J. E. Dahlberg, 1992, Methods of Enzymology: DNA Structure Part A: Synthesis and Physical Analysis of DNA Methods in Enzymology, Academic Press. Each of these general texts is herein incorporated by reference.

For the avoidance of doubt, Drosophila and vertebrate names are used interchangeably and all homologues are included within the scope of the invention.

Notch Signalling

As used herein, the expression “Notch signalling” is synonymous with the expression “the Notch signalling pathway” and refers to any one or more of the upstream or downstream events that result in, or from, (and including) activation of the Notch receptor.

Notch signalling directs binary cell fate decisions in the embryo. Notch was first described in Drosophila as a transmembrane protein that functions as a receptor for two different ligands, Delta and Serrate. Vertebrates express multiple Notch receptors and ligands. At least four Notch receptors (Notch-1, Notch-2, Notch-3 and Notch-4) have been identified to date in human cells.

Notch proteins are synthesized as single polypeptide precursors that undergo cleavage via a Furin-like convertase that yields two polypeptide chains that are further processed to form the mature receptor. The Notch receptor present in the plasma membrane comprises a heterodimer of two Notch proteolytic cleavage products, one comprising an N-terminal fragment consisting of a portion of the extracellular domain, the transmembrane domain and the intracellular domain, and the other comprising the majority of the extracellular domain. The proteolytic cleavage step of Notch to activate the receptor occurs and is mediated by a furin-like convertase.

Notch receptors are inserted into the membrane as disulphide-linked heterodimeric molecules consisting of an extracellular domain containing up to 36 epidermal growth factor (EGF)-like repeats and a transmembrane subunit that contains the cytoplasmic domain. The cytoplasmic domain of Notch contains six ankyrin-like repeats, a polyglutamine stretch (OPA) and a PEST sequence. A further domain termed RAM23 lies proximal to the ankyrin repeats and, like the ankyrin-like repeats, is involved in binding to a transcription factor, known as Suppressor of Hairless [Su(H)] in Drosophila and CBF1 in vertebrates (Tamura). The Notch ligands also display multiple EGF-like repeats in their extracellular domains together with a cysteine-rich DSL (Delta-Serrate Lag2) domain that is characteristic of all Notch ligands (Artavanis-Tsakonas).

The Notch receptor is activated by binding of extracellular ligands, such as Delta, Serrate and Scabrous, to the EGF-like repeats of Notch's extracellular domain. Delta requires cleavage for activation. It is cleaved by the ADAM disintegrin metalloprotease Kuzbanian at the cell surface, the cleavage event releasing a soluble and active form of Delta. An oncogenic variant of the human Notch-1 protein, also known as TAN-1, which has a truncated extracellular domain, is constitutively active and has been found to be involved in T-cell lymphoblastic leukemias.

The cdc10/ankyrin intracellular-domain repeats mediate physical interaction with intracellular signal transduction proteins. Most notably, the cdc10/ankyrin repeats interact with Suppressor of Hairless [Su(H)]. Su(H) is the Drosophila homologue of C-promoter binding factor-1 [CBF-1], a mammalian DNA binding protein involved in the Epstein-Barr virus-induced immortalization of B-cells. It has been demonstrated that, at least in cultured cells, Su(H) associates with the cdc10/ankyrin repeats in the cytoplasm and translocates into the nucleus upon the interaction of the Notch receptor with its ligand Delta on adjacent cells. Su(H) includes responsive elements found in the promoters of several genes and has been found to be a critical downstream protein in the Notch signalling pathway. The involvement of Su(H) in transcription is thought to be modulated by Hairless.

The intracellular domain of Notch (NotchIC) also has a direct nuclear function (Lieber). Recent studies have indeed shown that Notch activation requires that the six cdc10/ankyrin repeats of the Notch intracellular domain reach the nucleus and participate in transcriptional activation. The site of proteolytic cleavage on the intracellular tail of Notch has been identified between gly1743 and val1744 (termed site 3, or S3) (Schroeter). It is thought that the proteolytic cleavage step that releases the NotchIC for nuclear entry is dependent on Presenilin activity.

The intracellular domain has been shown to accumulate in the nucleus where it forms a transcriptional activator complex with the CSL family protein CBF1 (suppressor of hairless, Su(H) in Drosophila, Lag-2 in C. elegans) (Schroeter; Struhl). The NotchIC-CBF1 complexes then activate target genes, such as the bHLH proteins HES (hairy-enhancer of split like) 1 and 5 (Weinmaster). This nuclear function of Notch has also been shown for the mammalian Notch homologue (Lu).

NotchIC processing occurs only in response to binding of Notch ligands Delta or Serrate/Jagged. The post-translational modification of the nascent Notch receptor in the Golgi (Munro; Ju) appears, at least in part, to control which of the two types of ligand it interacts with on a cell surface. The Notch receptor is modified on its extracellular domain by Fringe, a glycosyl transferase enzyme that binds to the Notch/Lin motif. Fringe modifies Notch by adding O-linked fucose groups to the EGF-like repeats (Moloney; Bruckner). This modification by Fringe does not prevent ligand binding, but may influence ligand induced conformational changes in Notch. Furthermore, recent studies suggest that the action of Fringe modifies Notch to prevent it from interacting functionally with Serrate/Jagged ligands but allow it to preferentially interact with Delta (Panin; Hicks). Although Drosophila has a single Fringe gene, vertebrates are known to express multiple genes (Radical, Manic and Lunatic Fringes) (Irvine).

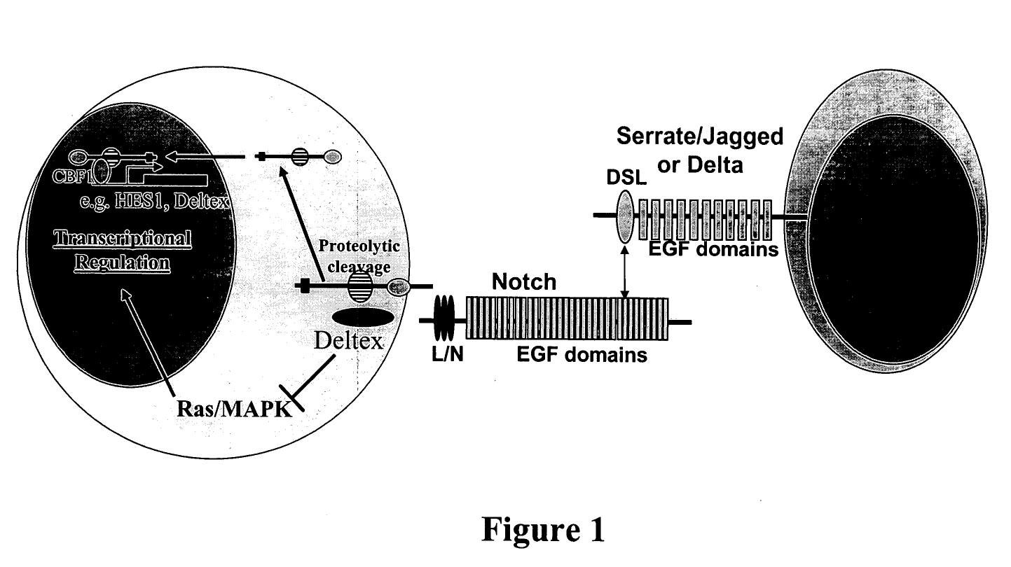

Thus, signal transduction from the Notch receptor can occur via different pathways. The better defined pathway involves proteolytic cleavage of the intracellular domain of Notch (NotchIC) that translocates to the nucleus and forms a transcriptional activator complex with the CSL family protein CBF1 (supressor of hairless, Su(H) in Drosophila, Lag-2 in C. elegans). NotchIC-CBF1 complexes then activate target genes, such as the bHLH proteins HES (hairy-enhancer of split like) 1 and 5. Notch can also signal in a CBF1-independent manner that involves the cytoplasmic zinc finger containing protein Deltex (FIG. 1). Unlike CBF1, Deltex does not move to the nucleus following Notch activation but instead can interact with Grb2 and modulate the Ras-Jnk signalling pathway.

As described above, several endogenous modulators of Notch are already known. These include, for example, the Notch ligands Delta and Serrate. An aim of the present invention is the detection of novel Notch signalling modulators.

Candidate Modulators

The term “modulate” as used herein refers to a change or alteration in the biological activity of the Notch signalling pathway or a target signalling pathway thereof. The term “modulator” may refer to antagonists or inhibitors of Notch signalling, i.e. compounds which block, at least to some extent, the normal biological activity of the Notch signalling pathway. Conveniently such compounds may be referred to herein as inhibitors or antagonists. Alternatively, the term “modulator” may refer to agonists of Notch signalling, i.e. compounds which stimulate or upregulate, at least to some extent, the normal biological activity of the Notch signalling pathway. Conveniently such compounds may be referred to as upregulators or agonists.

The term “candidate modulator” is used to describe any one or more molecule(s) which may be, or is suspected of being, capable of functioning as a modulator of Notch signalling. Said molecules may for example be organic “small molecules” or polypeptides. Suitably, candidate molecules comprise a plurality of, or a library of such molecules or polypeptides. These molecules may be derived from known modulators. “Derived from” means that the candidate modulator molecules preferably comprise polypeptides which have been fully or partially randomised from a starting sequence which is a known modulator of Notch signalling. Most preferably, candidate molecules comprise polypeptides which are at least about 40% homologous, more preferably at least about 60% homologous, even more preferably at least about 75% homologous or even more, for example about 85%, or about 90%, or even more than about 95% homologous to one or more known Notch modulator molecules, using the BLAST algorithm with the parameters as defined herein.

The candidate modulator of the present invention may be an organic compound or other chemical. In this embodiment, the candidate modulator will be an organic compound comprising two or more hydrocarbyl groups. Here, the term “hydrocarbyl group” means a group comprising at least C and H and may optionally comprise one or more other suitable substituents. Examples of such substituents may include halo-, alkoxy-, nitro-, an alkyl group, a cyclic group etc. In addition to the possibility of the substituents being a cyclic group, a combination of substituents may form a cyclic group. If the hydrocarbyl group comprises more than one C then those carbons need not necessarily be linked to each other. For example, at least two of the carbons may be linked via a suitable element or group. Thus, the hydrocarbyl group may contain hetero atoms. Suitable hetero atoms will be apparent to those skilled in the art and include, for instance, sulphur, nitrogen and oxygen. The candidate modulator may comprise at least one cyclic group. The cyclic group may be a polycyclic group, such as a non-fused polycyclic group. For some applications, the agent comprises at least the one of said cyclic groups linked to another hydrocarbyl group.

In one preferred embodiment, the candidate compound will be an amino acid sequence or a chemical derivative thereof, or a combination thereof. In another preferred embodiment, the candidate compound will be a nucleotide sequence, which may be a sense sequence or an anti-sense sequence. The candidate modulator may also be an antibody.

The term “antibody” includes intact molecules as well as fragments thereof, such as Fab, F(ab′)2, Fv and scFv which are capable of binding the epitopic determinant. These antibody fragments retain some ability to selectively bind with its antigen or receptor and include, for example:

(i) Fab, the fragment which contains a monovalent antigen-binding fragment of an antibody molecule can be produced by digestion of whole antibody with the enzyme papain to yield an intact light chain and a portion of one heavy chain;

(ii) Fab′, the fragment of an antibody molecule can be obtained by treating whole antibody with pepsin, followed by reduction, to yield an intact light chain and a portion of the heavy chain; two Fab′ fragments are obtained per antibody molecule;

(iii) F(ab′)₂, the fragment of the antibody that can be obtained by treating whole antibody with the enzyme pepsin without subsequent reduction; F(ab′)₂ is a dimer of two Fab′ fragments held together by two disulfide bonds;

(iv) scFv, including a genetically engineered fragment containing the variable region of a heavy and a light chain as a fused single chain molecule.

General methods of making these fragments are known in the art. (See for example, Harlow and Lane, Antibodies: A Laboratory Manual, Cold Spring Harbor Laboratory, New York (1988), which is incorporated herein by reference).

For example, antibodies against Notch and Notch ligands are described in U.S. Pat. No. 5,648,464, U.S. Pat. No. 5,849,869 and U.S. Pat. No. 6,004,924 (Yale University/Imperial Cancer Technology), the texts of which are herein incorporated by reference.

Antibodies generated against the Notch receptor are also described in WO 0020576 (the text of which is also incorporated herein by reference). For example, this document discloses generation of antibodies against the human Notch-1 EGF-like repeats 11 and 12. For example, in particular embodiments, WO 0020576 discloses a monoclonal antibody secreted by a hybridoma designated A6 having the ATCC Accession No. HB12654, a monoclonal antibody secreted by a hybridoma designated C11 having the ATCC Accession No. HB12656 and a monoclonal antibody secreted by a hybridoma designated F3 having the ATCC Accession No. HB12655.

An anti-human-Jagged1 antibody is available from R & D Systems, Inc, reference MAB12771 (Clone 188323).

Modulators may be synthetic compounds or natural isolated compounds.

By a protein which is for Notch signalling transduction is meant a molecule which participates in signalling through Notch receptors including activation of Notch, the downstream events of the Notch signalling pathway, transcriptional regulation of downstream target genes and other non-transcriptional downstream events (e.g. post-translational modification of existing proteins). More particularly, the protein is a domain that allows activation of target genes of the Notch signalling pathway, or a polynucleotide sequence which codes therefor.

A very important component of the Notch signalling pathway is Notch receptor/Notch ligand interaction. Thus Notch signalling may involve changes in expression, nature, amount or activity of Notch ligands or receptors or their resulting cleavage products. In addition, Notch signalling may involve changes in expression, nature, amount or activity of Notch signalling pathway membrane proteins or G-proteins or Notch signalling pathway enzymes such as proteases, kinases (e.g. serine/threonine kinases), phosphatases, ligases (e.g. ubiquitin ligases) or glycosyltransferases. Alternatively the signalling may involve changes in expression, nature, amount or activity of DNA binding elements such as transcription factors.

In the present invention Notch signalling means specific signalling, meaning that the signal detected results substantially or at least predominantly from the Notch signalling pathway, and preferably from Notch/Notch ligand interaction, rather than any other significant interfering or competing cause, such as cytokine signalling. In one embodiment the term “Notch signalling” excludes cytokine signalling. The Notch signalling pathway is described in more detail below.

Proteins or polypeptides may be in the form of the “mature” protein or may be a part of a larger protein such as a fusion protein or precursor. For example, it is often advantageous to include an additional amino acid sequence which contains secretory or leader sequences or pro-sequences (such as a HIS oligomer, immunoglobulin Fc, glutathione S-transferase, FLAG etc) to aid in purification. Likewise such an additional sequence may sometimes be desirable to provide added stability during recombinant production. In such cases the additional sequence may be cleaved (e.g. chemically or enzymatically) to yield the final product. In some cases, however, the additional sequence may also confer a desirable pharmacological profile (as in the case of IgFc fusion proteins) in which case it may be preferred that the additional sequence is not removed so that it is present in the final product as administered.

In one embodiment the Notch ligand which activates Notch may be expressed on a cell or cell membrane, suitably derived from a cell.

Candidate modulators may be synthetic compounds or natural isolated compounds. Various examples of such synthetic or natural modulators are listed below.

Candidate Modulators: Antagonists

Antagonists of Notch signalling will include any molecule which is capable of inhibiting Notch, the Notch signalling pathway or any one or more of the components of the Notch signalling pathway.

Candidate modulators for Notch signalling inhibition may be dominant negative versions of a compound capable of activating or transducing Notch signalling. Alternatively, the candidate modulator of Notch signalling will be capable of repressing a compound capable of activating or transducing Notch signalling. In a further alternative embodiment, the modulator will be an inhibitor of Notch signalling.

In a preferred embodiment, the modulator will be a polypeptide, or a polynucleotide encoding such a polypeptide, that decreases or interferes with the production of compounds that are capable of producing an increase in the expression of Notch ligand. Endogenous compounds of this type include Noggin, Chordin, Follistatin, Xnr3, fibroblast growth factors. Candidate modulators will include derivatives, fragments, variants, mimetics, analogues and homologues of any of the above.

Alternatively, the candidate modulator will be an antisense construct derived from a sense nucleotide sequence encoding a polypeptide selected from a Notch ligand and a polypeptide capable of up-regulating Notch ligand expression, such as Noggin, Chordin, Follistatin, Xnr3, fibroblast growth factors and derivatives, fragments, variants, mimetics, analogues and homologues thereof.

In another preferred embodiment the candidate modulator for Notch signalling inhibition will be a molecule which is capable of modulating Notch-Notch ligand interactions. A molecule may be considered to modulate Notch-Notch ligand interactions if it is capable of inhibiting the interaction of Notch with its ligands, preferably to an extent sufficient to provide therapeutic efficacy. In this embodiment the modulator may be a polypeptide, or a polynucleotide encoding such a polypeptide, selected from a Toll-like receptor, or a growth factor such as a BMP, a BMP receptor and activins, derivatives, fragments, variants, mimetics, homologues and analogues thereof. Preferably the modulator will decrease or interfere with the production of an agent that is capable of producing an increase in the expression of Notch ligand, such as Noggin, Chordin, Follistatin, Xnr3, fibroblast growth factors and derivatives, fragments, variants, mimetics homologues and analogues thereof.

Preferably when the modulator is a receptor or a nucleic acid sequence encoding a receptor, the receptor is activated. Thus, for example, when the modulator is a nucleic acid sequence, the receptor is constitutively active when expressed.

Modulators for Notch signalling inhibition also include downstream modulators of the Notch signalling pathway (such as Dsh, Numb and derivatives, fragments, variants, mimetics, homologues and analogues thereof), compounds that prevent expression of Notch target genes or induce expression of genes repressed by the Notch signalling pathway and dominant negative versions of Notch signalling transducer molecules (such as of NotchIC, Deltex and derivatives, fragments, variants, mimetics, homologues and analogues thereof). Proteins for Notch signalling inhibition will also include variants of the wild-type components of the Notch signalling pathway which have been modified in such a way that their presence blocks rather than transduces the signalling pathway. An example of such a modulator would be a Notch receptor which has been modified such that proteolytic cleavage of its intracellular domain is no longer possible.

Candidate Modulators: Agonists

Agonists of Notch signalling will include any molecule which is capable of up-regulating Notch, the Notch signalling pathway or any one or more of the components of the Notch signalling pathway. Candidate modulators for up-regulating the Notch signalling pathway include compounds capable of transducing or activating the Notch signalling pathway.

Modulators for Notch signalling transduction will include molecules which participate in signalling through Notch receptors including activation of Notch, the downstream events of the Notch signalling pathway, transcriptional regulation of downstream target genes and other non-transcriptional downstream events (e.g. post-translational modification of existing proteins). More particularly, such modulators will allow activation of target genes of the Notch signalling pathway.

According to one aspect of the present invention the modulator may be the Notch polypeptide or polynucleotide or a fragment, variant, derivative, mimetic or homologue thereof which retains the signalling transduction ability of Notch or an analogue of Notch which has the signalling transduction ability of Notch. By Notch, we mean Notch-1, Notch-2, Notch-3, Notch-4 and any other Notch homologues or analogues. Analogues of Notch include proteins from the Epstein Barr virus (EBV), such as EBNA2, BARF0 or LMP2A. In a particularly preferred embodiment the modulator may be the Notch intracellular domain (Notch IC) or a sub-fragment, variant, derivative, mimetic, analogue or homologue thereof.

Modulators for Notch signalling activation include molecules which are capable of activating Notch, the Notch signalling pathway or any one or more of the components of the Notch signalling pathway.

Such a modulator may be a dominant negative version of a Notch signalling repressor. In an alternative embodiment, the modulator will be capable of inhibiting a Notch signalling repressor. In a further alternative embodiment, the modulator for Notch signalling activation will be a positive activator of Notch signalling.

In a particular embodiment, the modulator will be capable of inducing or increasing Notch or Notch ligand expression. Such a molecule may be a nucleic acid sequence capable of inducing or increasing Notch or Notch ligand expression.

Endogenous agonists include Noggin, Chordin, Follistatin, Xnr3, fibroblast growth factors. Candidate modulators may therefore include derivatives, fragments, variants, mimetics, analogues and homologues thereof, or a polynucleotide encoding any one or more of the above.

In a preferred embodiment, the modulator may be a Notch ligand, or a polynucleotide encoding a Notch ligand. Notch ligands will typically be capable of binding to a Notch receptor polypeptide present in the membrane of a variety of mammalian cells, for example hemapoietic stem cells. Particular examples of mammalian Notch ligands identified to date include the Delta family, for example Delta or Delta-like 1 (Genbank Accession No. AF003522—Homo sapiens), Delta-3 (Genbank Accession No. AF084576—i Rattus norvegicus) and Delta-like 3 (Mus musculus) (Genbank Accession No. NM_{—}016941—Homo sapiens) and U.S. Pat. No. 6,121,045 (Millennium), Delta-4 (Genbank Accession Nos. AB043894 and AF 253468—Homo sapiens) and the Serrate family, for example Serrate-1 and Serrate-2—(WO97/01571, WO96/27610 and WO92/19734), Jagged-1 (Genbank Accession No. U73936—Homo sapiens) and Jagged-2 (Genbank Accession No. AF029778—Homo sapiens), and LAG-2. Homology between family members is extensive.

In one embodiment, the modulator may be a constitutively active Notch receptor or Notch intracellular domain, or a polynucleotide encoding such a receptor or intracellular domain.

In an alternative embodiment, the modulator of Notch signalling will act downstream of the Notch receptor. Thus, for example, the activator of Notch signalling may be a constitutively active Deltex polypeptide or a polynucleotide encoding such a polypeptide. Other endogenous downstream components of the Notch signalling pathway include Deltex-1, Deltex-2, Deltex-3, Suppressor of Deltex (SuDx), Numb and isoforms thereof, Numb associated Kinase (NAK), Notchless, Dishevelled (Dsh), emb5, Fringe genes (such as Radical, Lunatic and Manic), PON, LNX, Disabled, Numblike, Nur77, NFkB2, Mirror, Warthog, Engrailed-1 and Engrailed-2, Lip-1 and homologues thereof, the polypeptides involved in the Ras/MAPK cascade modulated by Deltex, polypeptides involved in the proteolytic cleavage of Notch such as Presenilin and polypeptides involved in the transcriptional regulation of Notch target genes. Candidate modulators of use in the present invention will therefore include constitutively active forms of any of the above, analogues, homologues, derivatives, variants, mimetics and fragments thereof.

Modulators for Notch signalling activation may also include any polypeptides expressed as a result of Notch activation and any polypeptides involved in the expression of such polypeptides, or polynucleotides encoding for such polypeptides.

Activation of Notch signalling may also be achieved by repressing inhibitors of the Notch signalling pathway. As such, candidate modulators will include molecules capable of repressing any Notch signalling inhibitors. Preferably the molecule will be a polypeptide, or a polynucleotide encoding such a polypeptide, that decreases or interferes with the production or activity of compounds that are capable of producing an decrease in the expression or activity of Notch, Notch ligands, or any downstream components of the Notch signalling pathway. In a preferred embodiment, the modulators will be capable of repressing polypeptides of the Toll-like receptor protein family, and growth factors such as the bone morphogenetic protein (BMP), BMP receptors and activins.

Preferably a modulator of Notch signalling will be in a multimerised form, and may preferably comprise a construct comprising at least 3, preferably at least 5, preferably at least 10, at least 30, or at least 50 or 100 or more modulators of Notch signalling.

For example, modulators of Notch signalling in the form of Notch ligand proteins/polypeptides coupled to particulate supports such as beads are described in WO 03/011317 (Lorantis) and in Lorantis' co-pending PCT application PCT/GB2003/001525 (filed on 4 Apr. 2003), the texts of which are hereby incorporated by reference (e.g. see in particular Examples 17, 18, 19 of PCT/GB2003/001525).

Modulators of Notch signalling in the form of Notch ligand proteins/polypeptides coupled to polymer supports are described in Lorantis Ltd's co-pending PCT application PCT/GB2003/003285 (filed on 1 Aug. 2003 claiming priority from GB 0218068.5), the text of which is herein incorporated by reference (e.g. see in particular Example 5 therein disclosing a dextran conjugate).

Polypeptides and Polynucleotides for Notch Signalling Transduction

The Notch signalling pathway directs binary cell fate decisions in the embryo. Notch was first described in Drosophila as a transmembrane protein that functions as a receptor for two different ligands, Delta and Serrate. Vertebrates express multiple Notch receptors and ligands (discussed below). At least four Notch receptors (Notch-1, Notch-2, Notch-3 and Notch-4) have been identified to date in human cells (see for example GenBank Accession Nos. AF308602, AF308601 and U95299—Homo sapiens).

Notch receptors are inserted into the membrane as disulphide-linked heterodimeric molecules consisting of an extracellular domain containing up to 36 epidermal growth factor (EGF)-like repeats [Notch 1/2=36, Notch 3=34 and Notch 4=29], 3 Cysteine Rich Repeats (Lin-Notch (L/N) repeats) and a transmembrane subunit that contains the cytoplasmic domain. The cytoplasmic domain of Notch contains six ankyrin-like repeats, a polyglutamine stretch (OPA) and a PEST sequence. A further domain termed RAM23 lies proximal to the ankyrin repeats and is involved in binding to a transcription factor, known as Suppressor of Hairless [Su(H)] in Drosophila and CBF1 in vertebrates (Tamura). The Notch ligands also display multiple EGF-like repeats in their extracellular domains together with a cysteine-rich DSL (Delta-Serrate Lag2) domain that is characteristic of all Notch ligands (Artavanis-Tsakonas).

S3 processing occurs only in response to binding of Notch ligands Delta or Serrate/Jagged. The post-translational modification of the nascent Notch receptor in the Golgi (Munro; Ju) appears, at least in part, to control which of the two types of ligand is expressed on a cell surface. The Notch receptor is modified on its extracellular domain by Fringe, a glycosyl transferase enzyme that binds to the Lin/Notch motif. Fringe modifies Notch by adding O-linked fucose groups to the EGF-like repeats (Moloney; Bruckner). This modification by Fringe does not prevent ligand binding, but may influence ligand induced conformational changes in Notch. Furthermore, recent studies suggest that the action of Fringe modifies Notch to prevent it from interacting functionally with Serrate/Jagged ligands but allow it to preferentially bind Delta (Panin; Hicks). Although Drosophila has a single Fringe gene, vertebrates are known to express multiple genes (Radical, Manic and Lunatic Fringes) (Irvine).

Signal transduction from the Notch receptor can occur via two different pathways (FIG. 1). The better defined pathway involves proteolytic cleavage of the intracellular domain of Notch (Notch IC) that translocates to the nucleus and forms a transcriptional activator complex with the CSL family protein CBF1 (suppressor of Hairless, Su(H) in Drosophila, Lag-2 in C. elegans). NotchIC-CBF1 complexes then activate target genes, such as the bHLH proteins HES (hairy-enhancer of split like) 1 and 5. Notch can also signal in a CBF1-independent manner that involves the cytoplasmic zinc finger containing protein Deltx. Unlike CBF1, Deltex does not move to the nucleus following Notch activation but instead can interact with Grb2 and modulate the Ras-JNK signalling pathway.

Target genes of the Notch signalling pathway include Deltex, genes of the Hes family (Hes-1 in particular), Enhancer of Split [E(spl)] complex genes, IL-10, CD-23, CD-4 and Dll-1.

Deltex, an intracellular docking protein, replaces Su(H) as it leaves its site of interaction with the intracellular tail of Notch. Deltex is a cytoplasmic protein containing a zinc-finger (Artavanis-Tsakonas; Osborne). It interacts with the ankyrin repeats of the Notch intracellular domain. Studies indicate that Deltex promotes Notch pathway activation by interacting with Grb2 and modulating the Ras-JNK signalling pathway (Matsuno). Deltex also acts as a docking protein which prevents Su(H) from binding to the intracellular tail of Notch (Matsuno). Thus, Su(H) is released into the nucleus where it acts as a transcriptional modulator. Recent evidence also suggests that, in a vertebrate B-cell system, Deltex, rather than the Su(H) homologue CBF1, is responsible for inhibiting E47 function (Ordentlich). Expression of Deltex is upregulated as a result of Notch activation in a positive feedback loop. The sequence of Homo sapiens Deltex (DTXI) mRNA may be found in GenBank Accession No. AF053700.

Hes-1 (Hairy-enhancer of Split-1) (Takebayashi) is a transcriptional factor with a basic helix-loop-helix structure. It binds to an important functional site in the CD4 silencer leading to repression of CD4 gene expression. Thus, Hes-1 is strongly involved in the determination of T-cell fate. Other genes from the Hes family include Hes-5 (mammalian Enhancer of Split homologue), the expression of which is also upregulated by Notch activation, and Hes-3. Expression of Hes-1 is upregulated as a result of Notch activation. The sequence of Mus musculus Hes-1 can be found in GenBank Accession No. D16464.

The E(spl) gene complex [E(spl)-C] (Leimeister) comprises seven genes of which only E(spl) and Groucho show visible phenotypes when mutant. E(spl) was named after its ability to enhance Split mutations, Split being another name for Notch. Indeed, E(spl)-C genes repress Delta through regulation of achaete-scute complex gene expression. Expression of E(spl) is upregulated as a result of Notch activation.

Interleukin-10 (IL-10) was first characterised in the mouse as a factor produced by Th2 cells which was able to suppress cytokine production by Th1 cells. It was then shown that IL-10 was produced by many other cell types including macrophages, keratinocytes, B cells, Th0 and Th1 cells. It shows extensive homology with the Epstein-Barr bcrfl gene which is now designated viral IL-10. Although a few immunostimulatory effects have been reported, it is mainly considered as an immunosuppressive cytokine. Inhibition of T cell responses by IL-10 is mainly mediated through a reduction of accessory functions of antigen presenting cells. IL-10 has notably been reported to suppress the production of numerous pro-inflammatory cytokines by macrophages and to inhibit co-stimulatory molecules and MHC class II expression. IL-10 also exerts anti-inflammatory effects on other myeloid cells such as neutrophils and eosinophils. On B cells, IL-10 influences isotype switching and proliferation. More recently, IL-10 was reported to play a role in the induction of regulatory T cells and as a possible mediator of their suppressive effect. Although it is not clear whether it is a direct downstream target of the Notch signalling pathway, its expression has been found to be strongly up-regulated coincident with Notch activation. The mRNA sequence of IL-10 may be found in GenBank ref. No. GI1041812.

CD-23 is the human leukocyte differentiation antigen CD23 (FCE2) which is a key molecule for B-cell activation and growth. It is the low-affinity receptor for IgE. Furthermore, the truncated molecule can be secreted, then functioning as a potent mitogenic growth factor. The sequence for CD-23 may be found in GenBank ref. No. GI1783344.

Dlx-1 (distalless-1) (McGuiness) expression is downregulated as a result of Notch activation. Sequences for Dlx genes may be found in GenBank Accession Nos. U51000-3.

CD-4 expression is downregulated as a result of Notch activation. A sequence for the CD-4 antigen may be found in GenBank Accession No. XM006966.

Other genes involved in the Notch signaling pathway, such as Numb, Mastermind and Dsh, and all genes the expression of which is modulated by Notch activation, are included in the scope of this invention.

Polypeptides and Polynucleotides for Notch Signalling Activation

Examples of mammalian Notch ligands identified to date include the Delta family, for example Delta-1 (Genbank Accession No. AF003522—Homo sapiens), Delta-3 (Genbank Accession No. AF084576—Rattus norvegicus) and Delta-like 3 (Mus musculus), the Serrate family, for example Serrate-1 and Serrate-2 (WO97/01571, WO96/27610 and WO92/19734), Jagged-1 and Jagged-2 (Genbank Accession No. AF029778—Homo sapiens), and LAG-2. Homology between family members is extensive. For example, human Jagged-2 has 40.6% identity and 58.7% similarity to Serrate.

Further homologues of known mammalian Notch ligands may be identified using standard techniques. By a “homologue” it is meant a gene product that exhibits sequence homology, either amino acid or nucleic acid sequence homology, to any one of the known Notch ligands, for example as mentioned above. Typically, a homologue of a known Notch ligand will be at least 20%, preferably at least 30%, identical at the amino acid level to the corresponding known Notch ligand over a sequence of at least 10, preferably at least 20, preferably at least 50, suitably at least 100 amino acids, or over the entire length of the Notch ligand. Techniques and software for calculating sequence homology between two or more amino acid or nucleic acid sequences are well known in the art. (See, for example, Ausubel et al., Current Protocols in Molecular Biology (1995), John Wiley & Sons, Inc. and databases maintained by the U.S. National Institutes of Health National Center for Biotechnology Information.)

Notch ligands identified to date have a diagnostic DSL domain (D. Delta, S. Serrate, L. Lag2) comprising 20 to 22 amino acids at the amino terminus of the protein and up to 14 or more EGF-like repeats on the extracellular surface. It is therefore preferred that homologues of Notch ligands also comprise a DSL domain at the N-terminus and up to 14 or more EGF-like repeats on the extracellular surface.

In addition, suitable homologues will be capable of binding to a Notch receptor. Binding may be assessed by a variety of techniques known in the art including in vitro binding assays.

Homologues of Notch ligands can be identified in a number of ways, for example by probing genomic or cDNA libraries with probes comprising all or part of a nucleic acid encoding a Notch ligand under conditions of medium to high stringency (for example 0.03M sodium chloride and 0.03M sodium citrate at from about 50° C. to about 60° C.). Alternatively, homologues may also be obtained using degenerate PCR which will generally use primers designed to target sequences within the variants and homologues encoding conserved amino acid sequences. The primers will contain one or more degenerate positions and will be used at stringency conditions lower than those used for cloning sequences with single sequence primers against known sequences.

Polypeptide substances may be purified from mammalian cells, obtained by recombinant expression in suitable host cells or obtained commercially. Alternatively, nucleic acid constructs encoding the polypeptides may be used. As a further example, overexpression of Notch or Notch ligand, such as Delta or Serrate, may be brought about by introduction of a nucleic acid construct capable of activating the endogenous gene, such as the Serrate or Delta gene. In particular, gene activation can be achieved by the use of homologous recombination to insert a heterologous promoter in place of the natural promoter, such as the Serrate or Delta promoter, in the genome of the target cell.

The activating molecule of the present invention may, in an alternative embodiment, be capable of modifying Notch-protein expression or presentation on the cell membrane or signalling pathways. Agents that enhance the presentation of a fully functional Notch-protein on the target cell surface include matrix metalloproteinases such as the product of the Kuzbanian gene of Drosophila (Dkuz) and other ADAMALYSIN gene family members.

Polypeptides and Polynucleotides for Notch Signalling Inhibition

Suitable nucleic acid sequences may include anti-sense constructs, for example nucleic acid sequences encoding antisense Notch ligand constructs as well as antisense constructs designed to reduce or inhibit the expression of upregulators of Notch ligand expression (see above). The antisense nucleic acid may be an oligonucleotide such as a synthetic single-stranded DNA. However, more preferably, the antisense is an antisense RNA produced in the patient's own cells as a result of introduction of a genetic vector. The vector is responsible for production of antisense RNA of the desired specificity on introduction of the vector into a host cell.

Preferably, the nucleic acid sequence for use in the present invention is capable of inhibiting Serrate and Delta, preferably Serrate 1 and Serrate 2 as well as Delta 1, Delta 3 and Delta 4 expression in APCs such as dendritic cells. In particular, the nucleic acid sequence may be capable of inhibiting Serrate expression but not Delta expression, or Delta but not Serrate expression in APCs or T cells. Alternatively, the nucleic acid sequence for use in the present invention is capable of inhibiting Delta expression in T cells such as CD4⁺ helper T cells or other cells of the immune system that express Delta (for example in response to stimulation of cell surface receptors). In particular, the nucleic acid sequence may be capable of inhibiting Delta expression but not Serrate expression in T cells. In a particularly preferred embodiment, the nucleic acid sequence is capable of inhibiting Notch ligand expression in both T cells and APC, for example Serrate expression in APCs and Delta expression in T cells.

Molecules for inhibition of Notch signalling will also include polypeptides, or polynucleotides which encode therefor, capable of modifying Notch-protein expression or presentation on the cell membrane or signalling pathways. Molecules that reduce or interfere with its presentation as a fully functional cell membrane protein may include MMP inhibitors such as hydroxymate-based inhibitors.

Other substances which may be used to reduce interaction between Notch and Notch ligands are exogenous Notch or Notch ligands or functional derivatives thereof. Such Notch ligand derivatives would preferably have the DSL domain at the N-terminus and up to about 14 or more, for example between about 3 to 8 EGF-like repeats on the extracellular surface. A peptide corresponding to the Delta/Serrate/LAG-2 domain of hJagged1 and supernatants from COS cells expressing a soluble form of the extracellular portion of hjagged1 was found to mimic the effect of Jagged1 in inhibiting Notch1 (Li).

Other Notch signalling pathway antagonists include antibodies which inhibit interactions between components of the Notch signalling pathway, e.g. antibodies to Notch ligands.

Whether a substance can be used for modulating Notch-Notch ligand expression may be determined using suitable screening assays.

Notch signalling can be monitored either through protein assays or through nucleic acid assays. Activation of the Notch receptor leads to the proteolytic cleavage of its cytoplasmic domain and the translocation thereof into the cell nucleus. The “detectable signal” referred to herein may be any detectable manifestation attributable to the presence of the cleaved intracellular domain of Notch. Thus, increased Notch signalling can be assessed at the protein level by measuring intracellular concentrations of the cleaved Notch domain. Activation of the Notch receptor also catalyses a series of downstream reactions leading to changes in the levels of expression of certain well defined genes. Thus, increased Notch signalling can be assessed at the nucleic acid level by say measuring intracellular concentrations of specific mRNAs. In one preferred embodiment of the present invention, the assay is a protein assay. In another preferred embodiment of the present invention, the assay is a nucleic acid assay.

The advantage of using a nucleic acid assay is that they are sensitive and that small samples can be analysed.

The intracellular concentration of a particular mRNA, measured at any given time, reflects the level of expression of the corresponding gene at that time. Thus, levels of mRNA of downstream target genes of the Notch signalling pathway can be measured in an indirect assay of the T-cells of the immune system. For example, an increase in levels of Deltex, Hes-1 and/or IL-10 mRNA may, for instance, indicate induced anergy while an increase in levels of IFN-γ mRNA, or in the levels of mRNA encoding cytokines such as IL-2, IL-5 and IL-13, may indicate improved responsiveness.

Many compounds identified according to the present invention may be lead compounds useful for drug development. Useful lead compounds include antibodies and peptides, and including intracellular antibodies expressed within the cell in a gene therapy context, which may be used as models for the development of peptide or low molecular weight therapeutics. In a preferred aspect of the invention, lead compounds and the Notch receptor or Notch ligand or other target peptides may be co-crystallised in order to facilitate the design of suitable low molecular weight compounds which mimic the interaction observed with the lead compound.

Any one or more of appropriate targets—such as an amino acid sequence and/or nucleotide sequence—may be used for identifying a compound capable of modulating the Notch signalling pathway and/or a targeting molecule in any of a variety of drug screening techniques. The target employed in such a test may be free in solution, affixed to a solid support, borne on a cell surface, or located intracellularly.

This invention also contemplates the use of competitive drug screening assays in which neutralising antibodies capable of binding a target specifically compete with a test compound for binding to a target.

Techniques are well known in the art for the screening and development of agents such as antibodies, peptidomimetics and small organic molecules which are capable of binding to and/or modulating components of the Notch signalling pathway. These include the use of phage display systems for expressing signalling proteins, and using a culture of transfected E. coli or other microorganism to produce the proteins for studies of potential binding and/or modulating compounds (see, for example, G. Cesarini, FEBS Letters, 307(1):66-70 (July 1992); H. Gram et al., J. Immunol. Meth., 161:169-176 (1993); and C. Summer et al., Proc. Natl. Acad. Sci., USA, 89:3756-3760 (May 1992)). Further library and screening techniques are described, for example, in U.S. Pat. No. 6,281,344 (Phylos).

With respect to Notch signalling and expression of cytokines, transcription factors, or other molecules discussed herein, the terms “increase,” “increased,” “decrease,” “decreased” and the like, are relative to a cell or cells in which signalling and/or expression has not been modulated, for example, as described herein.

Notch Ligands

As discussed above, Notch ligands comprise a number of distinctive domains. Some predicted/potential domain locations for various naturally occurring human Notch ligands (based on amino acid numbering in the precursor proteins) are shown below:

Human Delta 1

| Component | Amino acids | Proposed function/domain |
|---|---|---|
| SIGNAL | 1-17 | SIGNAL |
| CHAIN | 18-723 | DELTA-LIKE PROTEIN 1 |
| DOMAIN | 18-545 | EXTRACELLULAR |
| TRANSMEM | 546-568 | TRANSMEMBRANE |
| DOMAIN | 569-723 | CYTOPLASMIC |
| DOMAIN | 159-221 | DSL |
| DOMAIN | 226-254 | EGF-LIKE 1 |
| DOMAIN | 257-285 | EGF-LIKE 2 |
| DOMAIN | 292-325 | EGF-LIKE 3 |
| DOMAIN | 332-363 | EGF-LIKE 4 |
| DOMAIN | 370-402 | EGF-LIKE 5 |
| DOMAIN | 409-440 | EGF-LIKE 6 |
| DOMAIN | 447-478 | EGF-LIKE 7 |
| DOMAIN | 485-516 | EGF-LIKE 8 |

Human Delta 3

| Component | Amino acids | Proposed function/domain |
|---|---|---|
| DOMAIN | 158-248 | DSL |
| DOMAIN | 278-309 | EGF-LIKE 1 |
| DOMAIN | 316-350 | EGF-LIKE 2 |
| DOMAIN | 357-388 | EGF-LIKE 3 |
| DOMAIN | 395-426 | EGF-LIKE 4 |
| DOMAIN | 433-464 | EGF-LIKE 5 |

Human Delta 4

| Component | Amino acids | Proposed function/domain |
|---|---|---|
| SIGNAL | 1-26 | SIGNAL |
| CHAIN | 27-685 | DELTA-LIKE PROTEIN 4 |
| DOMAIN | 27-529 | EXTRACELLULAR |
| TRANSMEM | 530-550 | TRANSMEMBRANE |
| DOMAIN | 551-685 | CYTOPLASMIC |
| DOMAIN | 155-217 | DSL |
| DOMAIN | 218-251 | EGF-LIKE 1 |
| DOMAIN | 252-282 | EGF-LIKE 2 |
| DOMAIN | 284-322 | EGF-LIKE 3 |
| DOMAIN | 324-360 | EGF-LIKE 4 |
| DOMAIN | 362-400 | EGF-LIKE 5 |
| DOMAIN | 402-438 | EGF-LIKE 6 |
| DOMAIN | 440-476 | EGF-LIKE 7 |
| DOMAIN | 480-518 | EGF-LIKE 8 |

Human Jagged 1

| Component | Amino acids | Proposed function/domain |
|---|---|---|
| SIGNAL | 1-33 | SIGNAL |
| CHAIN | 34-1218 | JAGGED 1 |
| DOMAIN | 34-1067 | EXTRACELLULAR |
| TRANSMEM | 1068-1093 | TRANSMEMBRANE |
| DOMAIN | 1094-1218 | CYTOPLASMIC |
| DOMAIN | 167-229 | DSL |
| DOMAIN | 234-262 | EGF-LIKE 1 |
| DOMAIN | 265-293 | EGF-LIKE 2 |
| DOMAIN | 300-333 | EGF-LIKE 3 |
| DOMAIN | 340-371 | EGF-LIKE 4 |
| DOMAIN | 378-409 | EGF-LIKE 5 |
| DOMAIN | 416-447 | EGF-LIKE 6 |
| DOMAIN | 454-484 | EGF-LIKE 7 |
| DOMAIN | 491-522 | EGF-LIKE 8 |
| DOMAIN | 529-560 | EGF-LIKE 9 |
| DOMAIN | 595-626 | EGF-LIKE 10 |
| DOMAIN | 633-664 | EGF-LIKE 11 |
| DOMAIN | 671-702 | EGF-LIKE 12 |
| DOMAIN | 709-740 | EGF-LIKE 13 |
| DOMAIN | 748-779 | EGF-LIKE 14 |
| DOMAIN | 786-817 | EGF-LIKE 15 |
| DOMAIN | 824-855 | EGF-LIKE 16 |
| DOMAIN | 863-917 | VON WILLEBRAND FACTOR C |

Human Jagged 2

| Component | Amino acids | Proposed function/domain |
|---|---|---|
| SIGNAL | 1-26 | SIGNAL |
| CHAIN | 27-1238 | JAGGED 2 |
| DOMAIN | 27-1080 | EXTRACELLULAR |
| TRANSMEM | 1081-1105 | TRANSMEMBRANE |
| DOMAIN | 1106-1238 | CYTOPLASMIC |
| DOMAIN | 178-240 | DSL |
| DOMAIN | 249-273 | EGF-LIKE 1 |
| DOMAIN | 276-304 | EGF-LIKE 2 |
| DOMAIN | 311-344 | EGF-LIKE 3 |
| DOMAIN | 351-382 | EGF-LIKE 4 |
| DOMAIN | 389-420 | EGF-LIKE 5 |
| DOMAIN | 427-458 | EGF-LIKE 6 |
| DOMAIN | 465-495 | EGF-LIKE 7 |
| DOMAIN | 502-533 | EGF-LIKE 8 |
| DOMAIN | 540-571 | EGF-LIKE 9 |
| DOMAIN | 602-633 | EGF-LIKE 10 |
| DOMAIN | 640-671 | EGF-LIKE 11 |
| DOMAIN | 678-709 | EGF-LIKE 12 |
| DOMAIN | 716-747 | EGF-LIKE 13 |
| DOMAIN | 755-786 | EGF-LIKE 14 |
| DOMAIN | 793-824 | EGF-LIKE 15 |
| DOMAIN | 831-862 | EGF-LIKE 16 |
| DOMAIN | 872-949 | VON WILLEBRAND FACTOR C |

DSL Domain

A typical DSL domain may include most or all of the following consensus amino acid sequence (SEQ ID NO:23):

Cys Xaa Xaa Xaa Xaa Xaa Xaa Xaa Xaa Cys Xaa Xaa
Xaa Cys Xaa Xaa Xaa Xaa Xaa Xaa Xaa Xaa Xaa Xaa
Xaa Cys Xaa Xaa Xaa Xaa Xaa Xaa Xaa Cys Xaa Xaa
Xaa Xaa Xaa Xaa Xaa Xaa Cys

Preferably the DSL domain may include most or all of the following consensus amino acid sequence (SEQ ID NO:24):

Cys Xaa Xaa Xaa ARO ARO Xaa Xaa Xaa Cys Xaa Xaa Xaa Cys BAS NOP BAS ACM ACM Xaa ARO NOP ARO Xaa Xaa Cys Xaa Xaa Xaa NOP Xaa Xaa Xaa Cys Xaa Xaa NOP ARO Xaa NOP Xaa Xaa Cys

wherein:

ARO is an aromatic amino acid residue, such as tyrosine, phenylalanine, tryptophan or histidine;

NOP is a non-polar amino acid residue such as glycine, alanine, proline, leucine, isoleucine or valine;

BAS is a basic amino acid residue such as arginine or lysine; and

ACM is an acid or amide amino acid residue such as aspartic acid, glutamic acid, asparagine or glutamine.

Preferably the DSL domain may include most or all of the following consensus amino acid sequence (SEQ ID NO:25):

Cys Xaa Xaa Xaa Tyr Tyr Xaa Xaa Xaa Cys Xaa Xaa
Xaa Cys Arg Pro Arg Asx Asp Xaa Phe Gly His Xaa
Xaa Cys Xaa Xaa Xaa Gly Xaa Xaa Xaa Cys Xaa Xaa
Gly Trp Xaa Gly Xaa Xaa Cys

(wherein Xaa may be any amino acid and Asx is either aspartic acid or asparagine).

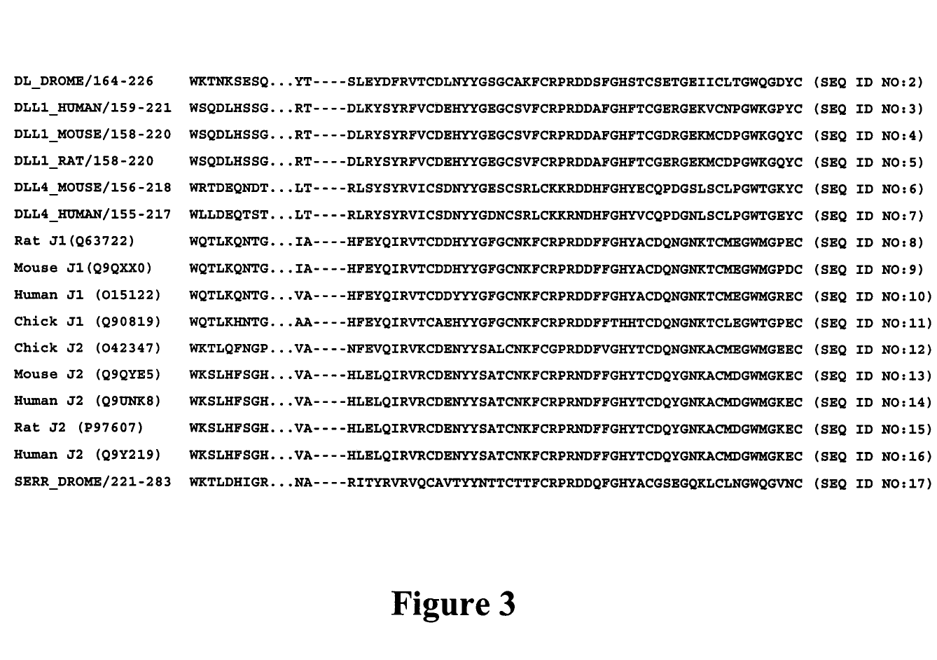

An alignment of DSL domains from Notch ligands from various sources is shown in FIG. 3.

The DSL domain used may be derived from any suitable species, including for example Drosophila, Xenopus, rat, mouse or human. Preferably the DSL domain is derived from a vertebrate, preferably a mammalian, preferably a human Notch ligand sequence.

Suitably, for example, a DSL domain for use in the present invention may have at least about 30%, preferably at least about 50%, preferably at least about 60%, preferably at least about 70%, preferably at least about 80%, preferably at least about 90%, preferably at least about 95% amino acid sequence identity to the DSL domain of human Jagged 1.

Alternatively a DSL domain for use in the present invention may, for example, have at least about 30%, preferably at least about 50%, preferably at least about 60%, preferably at least about 70%, preferably at least about 80%, preferably at least about 90%, preferably at least about 95% amino acid sequence identity to the DSL domain of human Jagged 2.

Alternatively a DSL domain for use in the present invention may, for example, have at least about 30%, preferably at least about 50%, preferably at least about 60%, preferably at least about 70%, preferably at least about 80%, preferably at least about 90%, preferably at least about 95% amino acid sequence identity to the DSL domain of human Delta 1.

Alternatively a DSL domain for use in the present invention may, for example, have at least about 30%, preferably at least about 50%, preferably at least about 60%, preferably at least about 70%, preferably at least about 80%, preferably at least about 90%, preferably at least about 95% amino acid sequence identity to the DSL domain of human Delta 3.

Alternatively a DSL domain for use in the present invention may, for example, have at least about 30%, preferably at least about 50%, preferably at least about 60%, preferably at least about 70%, preferably at least about 80%, preferably at least about 90%, preferably at least about 95% amino acid sequence identity to the DSL domain of human Delta 4.

EGF-Like Domain

The EGF-like motif has been found in a variety of proteins, as well as EGF and Notch and Notch ligands, including those involved in the blood clotting cascade (Furie and Furie, 1988, Cell 53: 505-518). For example, this motif has been found in extracellular proteins such as the blood clotting factors 1× and X (Rees et al., 1988, EMBO J. 7:2053-2061; Furie and Furie, 1988, Cell 53: 505-518), in other Drosophila genes (Knust et al., 1987 EMBO J. 761-766; Rothberg et al., 1988, Cell 55:1047-1059), and in some cell-surface receptor proteins, such as thrombomodulin (Suzuki et al., 1987, EMBO J. 6:1891-1897) and LDL receptor (Sudhof et al., 1985, Science 228:815-822). A protein binding site has been mapped to the EGF repeat domain in thrombomodulin and urokinase (Kurosawa et al., 1988, J. Biol. Chem 263:5993-5996; Appella et al., 1987, J. Biol. Chem. 262:4437-4440).

As reported by PROSITE the EGF domain typically includes six cysteine residues which have been shown (in EGF) to be involved in disulfide bonds. The main structure is proposed, but not necessarily required, to be a two-stranded beta-sheet followed by a loop to a C-terminal short two-stranded sheet. Subdomains between the conserved cysteines strongly vary in length as shown in the following schematic representation of the EGF-like domain (SEQ ID NO:26):

```
                                +---------------------+ +--------------------------+
                                | | | |
x(4)-C-x(0, 48)-C-x(3, 12)-C-x(1, 70)-C-x(1, 6)-C-x(2)-G-a-x(0, 21)-G-x(2)-C-x
          | |
          +---------------------+
wherein:
‘C’: conserved cysteine involved in a disulfide bond.
‘G’: often conserved glycine
‘a’: often conserved aromatic amino acid
‘x’: any residue
```

The region between the 5th and 6th cysteine contains two conserved glycines of which at least one is normally present in most EGF-like domains.

The EGF-like domain used may be derived from any suitable species, including for example Drosophila, Xenopus, rat, mouse or human. Preferably the EGF-like domain is derived from a vertebrate, preferably a mammalian, preferably a human Notch ligand sequence.

Suitably, for example, an EGF-like domain for use in the present invention may have at least about 30%, preferably at least about 50%, preferably at least about 60%, preferably at least about 70%, preferably at least about 80%, preferably at least about 90%, preferably at least about 95% amino acid sequence identity to an EGF-like domain of human Jagged 1.

Alternatively an EGF-like domain for use in the present invention may, for example, have at least about 30%, preferably at least about 50%, preferably at least about 60%, preferably at least about 70%, preferably at least about 80%, preferably at least about 90%, preferably at least about 95% amino acid sequence identity to an EGF-like domain of human Jagged 2.

Alternatively an EGF-like domain for use in the present invention may, for example, have at least about 30%, preferably at least about 50%, preferably at least about 60%, preferably at least about 70%, preferably at least about 80%, preferably at least about about 90%, preferably at least about 95% amino acid sequence identity to an EGF-like domain of human Delta 1.

Alternatively an EGF-like domain for use in the present invention may, for example, have at least about 30%, preferably at least about 50%, preferably at least about 60%, preferably at least about 70%, preferably at least about 80%, preferably at least about 90%, preferably at least about 95% amino acid sequence identity to an EGF-like domain of human Delta 3.

Alternatively an EGF-like domain for use in the present invention may, for example, have at least about 30%, preferably at least about 50%, preferably at least about 60%, preferably at least about 70%, preferably at least about 80%, preferably at least about 90%, preferably at least about 95% amino acid sequence identity to an EGF-like domain of human Delta 4.

As a practical matter, whether any particular amino acid sequence is at least X % identical to another sequence can be determined conventionally using known computer programs. For example, the best overall match between a query sequence and a subject sequence, also referred to as a global sequence alignment, can be determined using a program such as the FASTDB computer program based on the algorithm of Brutlag et al. (Comp. App. Biosci. (1990) 6:237-245). In a sequence alignment the query and subject sequences are either both nucleotide sequences or both amino acid sequences. The result of the global sequence alignment is given as percent identity. Suitable parameters used in a FASTDB amino acid alignment are: Matrix=PAM 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter.

The term “Notch ligand N-terminal domain” means the part of a Notch ligand sequence from the N-terminus to the start of the DSL domain. It will be appreciated that this term includes sequence variants, fragments, derivatives and mimetics having activity corresponding to naturally occurring domains.

Suitably, for example, a Notch ligand N-terminal domain for use in the present invention may have at least about 30%, preferably at least about 50%, preferably at least about 60%, preferably at least about 70%, preferably at least about 80%, preferably at least about 90%, preferably at least about 95% amino acid sequence identity to a Notch ligand N-terminal domain of human Jagged 1.

Alternatively a Notch ligand N-terminal domain for use in the present invention may, for example, have at least about 30%, preferably at least about 50%, preferably at least about 60%, preferably at least about 70%, preferably at least about 80%, preferably at least about 90%, preferably at least about 95% amino acid sequence identity to a Notch ligand N-terminal domain of human Jagged 2.

Alternatively a Notch ligand N-terminal domain for use in the present invention may, for example, have at least about 30%, preferably at least about 50%, preferably at least about 60%, preferably at least about 70%, preferably at least about 80%, preferably at least about 90%, preferably at least about 95% amino acid sequence identity to a Notch ligand N-terminal domain of human Delta 1.

Alternatively a Notch ligand N-terminal domain for use in the present invention may, for example, have at least about 30%, preferably at least about 50%, preferably at least about 60%, preferably at least about 70%, preferably at least about 80%, preferably at least about 90%, preferably at least about 95% amino acid sequence identity to a Notch ligand N-terminal domain of human Delta 3.

Alternatively a Notch ligand N-terminal domain for use in the present invention may, for example, have at least about 30%, preferably at least about 50%, preferably at least about 60%, preferably at least about 70%, preferably at least about 80%, preferably at least about 90%, preferably at least about 95% amino acid sequence identity to a Notch ligand N-terminal domain of human Delta 4.

The term “heterologous amino acid sequence” or “heterologous nucleotide sequence” as used herein means a sequence which is not found in the native sequence (e.g. in the case of a Notch ligand sequence is not found in the native Notch ligand sequence) or its coding sequence. Typically, for example, such a sequence may be an IgFc domain or a tag such as a V5His tag.

Polypeptide Sequences

As used herein, the term “polypeptide” is synonymous with the term “amino acid sequence” and/or the term “protein”. In some instances, the term “polypeptide” is synonymous with the term “peptide”.

“Peptide” usually refers to a short amino acid sequence that is 10 to 40 amino acids long, preferably 10 to 35 amino acids.

The polypeptide sequence may be prepared and isolated from a suitable source, or it may be made synthetically or it may be prepared by use of recombinant DNA techniques.

Polynucleotide Sequences

As used herein, the term “polynucleotide sequence” is synonymous with the term “polynucleotide” and/or the term “nucleotide sequence”.

The polynucleotide sequence may be DNA or RNA of genomic or synthetic or of recombinant origin. They may also be cloned by standard techniques. The polynucleotide sequence may be double-stranded or single-stranded whether representing the sense or antisense strand or combinations thereof.

“Polynucleotide” refers to a polymeric form of nucleotides of at least 10 bases in length and up to 1,000-5,000 bases or even more. Longer polynucleotide sequences will generally be produced using recombinant means, for example using a PCR (polymerase chain reaction) cloning techniques. This will involve making a pair of primers (e.g. of about 15 to 30 nucleotides) flanking a region of the targeting sequence which it is desired to clone, bringing the primers into contact with mRNA or cDNA obtained from an animal or human cell, performing a polymerase chain reaction (PCR) under conditions which bring about amplification of the desired region, isolating the amplified fragment (e.g. by purifying the reaction mixture on an agarose gel) and recovering the amplified DNA. The primers may be designed to contain suitable restriction enzyme recognition sites so that the amplified DNA can be cloned into a suitable cloning vector.

The nucleic acid may be RNA or DNA and is preferably DNA. Where it is RNA, manipulations may be performed via cDNA intermediates. Generally, a nucleic acid sequence encoding the first region will be prepared and suitable restriction sites provided at the 5′ and/or 3′ ends. Conveniently the sequence is manipulated in a standard laboratory vector, such as a plasmid vector based on pBR322 or pUC19 (see below). Reference may be made to Molecular Cloning by Sambrook et al. (Cold Spring Harbor, 1989) or similar standard reference books for exact details of the appropriate techniques.

Sources of nucleic acid may be ascertained by reference to published literature or databanks such as GenBank. Nucleic acid encoding the desired first or second sequences may be obtained from academic or commercial sources where such sources are willing to provide the material or by synthesising or cloning the appropriate sequence where only the sequence data are available. Generally this may be done by reference to literature sources which describe the cloning of the gene in question.

Alternatively, where limited sequence data is available or where it is desired to express a nucleic acid homologous or otherwise related to a known nucleic acid, exemplary nucleic acids can be characterised as those nucleotide sequences which hybridise to the nucleic acid sequences known in the art.

The polynucleotide sequence may comprise, for example, a protein-encoding domain, an antisense sequence or a functional motif such as a protein-binding domain and includes variants, derivatives, analogues and fragments thereof. The term also refers to polypeptides encoded by the nucleotide sequence.

The nucleotide sequences such as a DNA polynucleotides useful in the invention may be produced recombinantly, synthetically, or by any means available to those of skill in the art. They may also be cloned by standard techniques.

In general, primers will be produced by synthetic means, involving a step wise manufacture of the desired nucleic acid sequence one nucleotide at a time. Techniques for accomplishing this using automated techniques are readily available in the art.

Longer nucleotide sequences will generally be produced using recombinant means, for example using a PCR (polymerase chain reaction) cloning techniques. This will involve making a pair of primers (e.g. of about 15 to 30 nucleotides) flanking a region of the targeting sequence which it is desired to clone, bringing the primers into contact with mRNA or cDNA obtained from an animal or human cell, performing a polymerase chain reaction (PCR) under conditions which bring about amplification of the desired region, isolating the amplified fragment (e.g. by purifying the reaction mixture on an agarose gel) and recovering the amplified DNA. The primers may be designed to contain suitable restriction enzyme recognition sites so that the amplified DNA can be cloned into a suitable cloning vector.

For recombinant production, host cells can be genetically engineered to incorporate expression systems or polynucleotides of the invention. Introduction of a polynucleotide into the host cell can be effected by methods described in many standard laboratory manuals, such as Davis et al. and Sambrook et al., such as calcium phosphate transfection, DEAE-dextran mediated transfection, transfection, microinjection, cationic lipid-mediated transfection, electroporation, transduction, scrape loading, ballistic introduction and infection. It will be appreciated that such methods can be employed in vitro or in vivo as drug delivery systems.

Representative examples of appropriate hosts include bacterial cells, such as streptococci, staphylococci, E. coli, streptomyces and Bacillus subtilis cells; fungal cells, such as yeast cells and Aspergillus cells; insect cells such as Drosophila S2 and Spodoptera Sf9 cells; animal cells such as CHO, COS, NSO, HeLa, C127, 3T3, BHK, 293 and Bowes melanoma cells; and plant cells.

A great variety of expression systems can be used to produce a polypeptide useful in the present invention. Such vectors include, among others, chromosomal, episomal and virus-derived vectors, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. The expression system constructs may contain control regions that regulate as well as engender expression. Generally, any system or vector suitable to maintain, propagate or express polynucleotides and/or to express a polypeptide in a host may be used for expression in this regard. The appropriate DNA sequence may be inserted into the expression system by any of a variety of well-known and routine techniques, such as, for example, those set forth in Sambrook et al.

For secretion of the translated protein into the lumen of the endoplasmic reticulum, into the periplasmic space or into the extracellular environment, appropriate secretion signals may be incorporated into the expressed polypeptide. These signals may be endogenous to the polypeptide or they may be heterologous signals.

Active agents for use in the invention can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography is employed for purification. Well known techniques for refolding protein may be employed to regenerate active conformation when the polypeptide is denatured during isolation and/or purification.

Variants, Derivatives, Analogues, Homologues and Fragments

In addition to the specific polypeptide and polynucleotide sequences mentioned herein, the present invention also encompasses the use of variants, derivatives, analogues, homologues, mimetics and fragments thereof.

In the context of the present invention, a variant of any given sequence is a sequence in which the specific sequence of residues (whether amino acid or nucleic acid residues) has been modified in such a manner that the polypeptide or polynucleotide in question retains at least one of its endogenous functions. A variant sequence can be modified by addition, deletion, substitution modification replacement and/or variation of at least one residue present in the naturally-occurring protein.

The term “derivative” as used herein, in relation to proteins or polypeptides of the present invention includes any substitution of, variation of, modification of, replacement of, deletion of and/or addition of one (or more) amino acid residues from or to the sequence providing that the resultant protein or polypeptide retains at least one of its endogenous functions.

The term “analogue” as used herein, in relation to polypeptides or polynucleotides, includes any polypeptide or polynucleotide which retains at least one of the functions of the endogenous polypeptide or polynucleotide but generally has a different evolutionary origin thereto.

The term “mimetic” as used herein, in relation to polypeptides or polynucleotides, refers to a chemical compound that possesses at least one of the endogenous functions of the polypeptide or polynucleotide which it mimics.

Typically, amino acid substitutions may be made, for example from 1, 2 or 3 to 10 or 20 substitutions provided that the modified sequence retains the required transport activity or ability to modulate Notch signalling. Amino acid substitutions may include the use of non-naturally occurring analogues.

Proteins of use in the present invention may also have deletions, insertions or substitutions of amino acid residues which produce a silent change and result in a functionally equivalent protein. Deliberate amino acid substitutions may be made on the basis of similarity in polarity, charge, solubility, hydrophobicity, hydrophilicity, and/or the amphipathic nature of the residues as long as the transport or modulation function is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

For ease of reference, the one and three letter codes for the main naturally occurring amino acids (and their associated codons) are set out below:

| | | | |
|---|---|---|---|
| Symbol | 3-letter | Meaning | Codons |

| | | | |
|---|---|---|---|
| A | Ala | Alanine | GCT, GCC, GCA, GCG |
| B | Asp,Asn | Aspartic, | GAT, GAC, AAT, AAC |
| | | Asparagine | |
| C | Cys | Cysteine | TGT, TGC |
| D | Asp | Aspartic | GAT, GAC |
| E | Glu | Glutamic | GAA, GAG |
| F | Phe | Phenylalanine | TTT, TTC |
| G | Gly | Glycine | GGT, GGC, GGA, GGG |
| H | His | Histidine | CAT, CAC |
| I | Ile | Isoleucine | ATT, ATC, ATA |
| K | Lys | Lysine | AAA, AAG |
| L | Leu | Leucine | TTG, TTA, CTT, CTC, |
| | | | CTA, CTG |
| M | Met | Methionine | ATG |
| N | Asn | Asparagine | AAT, AAC |
| P | Pro | Proline | CCT, CCC, CCA, CCG |
| o | Gln | Glutamine | CAA, CAG |
| R | Arg | Arginine | CGT, CGC, CGA, CGG, |
| | | | AGA, AGG |
| S | Ser | Serine | TCT, TCC, TCA, TCG, |
| | | | AGT, AGC |
| T | Thr | Threonine | ACT, ACC, ACA, ACG |
| V | Val | Valine | GTT, GTC, GTA, GTG |
| W | Trp | Tryptophan | TGG |
| X | Xxx | Unknown | |
| Y | Tyr | Tyrosine | TAT, TAC |
| Z | Glu, Gln | Glutamic, | GAA, GAG, CAA, CAG |
| | | Glutamine | |
| * | End | Terminator | TAA, TAG, TGA |

Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

As used herein, the term “protein” includes single-chain polypeptide molecules as well as multiple-polypeptide complexes where individual constituent polypeptides are linked by covalent or non-covalent means. As used herein, the terms “polypeptide” and “peptide” refer to a polymer in which the monomers are amino acids and are joined together through peptide or disulfide bonds. The terms subunit and domain may also refer to polypeptides and peptides having biological function.

“Fragments” are also variants and the term typically refers to a selected region of the polypeptide or polynucleotide that is of interest either functionally or, for example, in an assay. “Fragment” thus refers to an amino acid or nucleic acid sequence that is a portion of a full-length polypeptide or polynucleodtide.

Such variants may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site. The flanking regions will contain convenient restriction sites corresponding to sites in the naturally-occurring sequence so that the sequence may be cut with the appropriate enzyme(s) and the synthetic DNA ligated into the cut. The DNA is then expressed in accordance with the invention to make the encoded protein. These methods are only illustrative of the numerous standard techniques known in the art for manipulation of DNA sequences and other known techniques may also be used.

Polynucleotide variants will preferably comprise codon optimised sequences. Codon optimisation is known in the art as a method of enhancing RNA stability and therefor gene expression. The redundancy of the genetic code means that several different codons may encode the same amino-acid. For example, Leucine, Arginine and Serine are each encoded by six different codons. Different organisms show preferences in their use of the different codons. Viruses such as HIV, for instance, use a large number of rare codons. By changing a nucleotide sequence such that rare codons are replaced by the corresponding commonly used mammalian codons, increased expression of the sequences in mammalian target cells can be achieved. Codon usage tables are known in the art for mammalian cells, as well as for a variety of other organisms. Preferably, at least part of the sequence is codon optimised. Even more preferably, the sequence is codon optimised in its entirety.

As used herein, the term “homology” can be equated with “identity”. An homologous sequence will be taken to include an amino acid sequence which may be at least 75, 85 or 90% identical, preferably at least 95 or 98% identical. In particular, homology should typically be considered with respect to those regions of the sequence (such as amino acids at positions 51, 56 and 57) known to be essential for an activity. Although homology can also be considered in terms of similarity (i.e. amino acid residues having similar chemical properties/functions), in the context of the present invention it is preferred to express homology in terms of sequence identity.

Homology comparisons can be conducted by eye, or more usually, with the aid of readily available sequence comparison programs. These commercially available computer programs can calculate % homology between two or more sequences.

Percent homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package (see below) the default gap penalty for amino acid sequences is −12 for a gap and −4 for each extension.

Calculation of maximum % homology therefor firstly requires the production of an optimal alignment, taking into consideration gap penalties. A suitable computer program for carrying out such an alignment is the GCG Wisconsin Bestfit package (Devereux). Examples of other software than can perform sequence comparisons include, but are not limited to, the BLAST package, FASTA (Atschul) and the GENEWORKS suite of comparison tools. Both BLAST and FASTA are available for offline and online searching. However it is preferred to use the GCG Bestfit program.

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). It is preferred to use the public default values for the GCG package, or in the case of other software, the default matrix, such as BLOSUM62.

Once the software has produced an optimal alignment, it is possible to calculate % homology, preferably % sequence identity. The software typically does this as part of the sequence comparison and generates a numerical result.

Nucleotide sequences which are homologous to or variants of sequences of use in the present invention can be obtained in a number of ways, for example by probing DNA libraries made from a range of sources. In addition, other viral/bacterial, or cellular homologues particularly cellular homologues found in mammalian cells (e.g. rat, mouse, bovine and primate cells), may be obtained and such homologues and fragments thereof in general will be capable of selectively hybridising to the sequences shown in the sequence listing herein. Such sequences may be obtained by probing cDNA libraries made from or genomic DNA libraries from other animal species, and probing such libraries with probes comprising all or part of the reference nucleotide sequence under conditions of medium to high stringency. Similar considerations apply to obtaining species homologues and allelic variants of the amino acid and/or nucleotide sequences useful in the present invention.

Variants and strain/species homologues may also be obtained using degenerate PCR which will use primers designed to target sequences within the variants and homologues encoding conserved amino acid sequences within the sequences of use in the present invention. Conserved sequences can be predicted, for example, by aligning the amino acid sequences from several variants/homologues. Sequence alignments can be performed using computer software known in the art. For example the GCG Wisconsin PileUp program is widely used. The primers used in degenerate PCR will contain one or more degenerate positions and will be used at stringency conditions lower than those used for cloning sequences with single sequence primers against known sequences.

Alternatively, such nucleotide sequences may be obtained by site directed mutagenesis of characterised sequences. This may be useful where for example silent codon changes are required to sequences to optimise codon preferences for a particular host cell in which the nucleotide sequences are being expressed. Other sequence changes may be desired in order to introduce restriction enzyme recognition sites, or to alter the activity of the polynucleotide or encoded polypeptide.

In a first step of the method of the present invention, any one or more of the above candidate modulators is brought into contact with a cell of the immune system. Cells of the immune system of use in the present invention are described below.

By Notch, we mean Notch-1, Notch-2, Notch-3 or Notch-4 and any other Notch homologues or analogues. The term “Notch IC” includes the full intracellular domain of Notch or an active portion of this domain. For example, the sequence may be a sequence comprising or coding for at least amino acids 1848 to 2202 of human Notch1 or a sequence having at least 70%, preferably at least 75%, preferably at least 80%, preferably at least 85%, preferably at least 90%, preferably at least 95% amino acid sequence similarity or identity with this sequence. The sequence may also suitably be derived from human Notch2, Notch3 or Notch4. Suitably the Notch sequence comprises at least a Notch Ankyrin repeat domain and optionally a Notch LNR domain, Notch RAM domain, Notch OPA domain and/or Notch PEST sequence.

Cells of the Immune System

Cells of use in the present invention are cells of the immune system capable of transducing the Notch signalling pathway.

Most preferably the cells of use in the present invention are T-cells. These include, but are not limited to, CD4⁺ and CD8⁺ mature T cells, immature T cells of peripheral or thymic origin and NK-T cells.

Alternatively, the cells will be antigen-presenting cells (APCs). APCs include dendritic cells (DCs) such as interdigitating DCs or follicular DCs, Langerhans cells, PBMCs, macrophages, B-lymphocytes, T-lymphocytes, or other cell types such as epithelial cells, fibroblasts or endothelial cells, constitutively expressing or activated to express a MHC Class II molecules on their surfaces. Precursors of APCs include CD34⁺ cells, monocytes, fibroblasts and endothelial cells. The APCs or precursors may be modified by the culture conditions or may be genetically modified, for instance by transfection of one or more genes.

The T cells or APCs may be isolated from a patient, or from a donor individual or another individual. The cells are preferably mammalian cells such as human or mouse cells. Preferably the cells are of human origin. The APC or precursor APC may be provided by a cell proliferating in culture such as an established cell line or a primary cell culture. Examples include hybridoma cell lines, L-cells and human fibroblasts such as MRC-5. Preferred cell lines for use in the present invention include Jurkat, H9, CEM and EL4 T-cells; long-term T-cell clones such as human HA1.7 or mouse D10 cells; T-cell hybridomas such as DO11.10 cells; macrophage-like cells such as U937 or THP1 cells; B-cell lines such as EBV-transformed cells such as Raji, A20 and M1 cells.

Dendritic cells (DCs) can be isolated/prepared by a number of means, for example they can either be purified directly from peripheral blood, or generated from CD34⁺ precursor cells for example after mobilisation into peripheral blood by treatment with GM-CSF, or directly from bone marrow. From peripheral blood, adherent precursors can be treated with a GM-CSF/IL-4 mixture (Inaba et al.), or from bone marrow, non-adherent CD34⁺ cells can be treated with GM-CSF and TNF-α (Caux et al.). DCs can also be routinely prepared from the peripheral blood of human volunteers, similarly to the method of Sallusto and Lanzavecchia J Exp Med (1994) 179(4) 1109-18 using purified peripheral blood mononucleocytes (PBMCs) and treating 2 hour adherent cells with GM-CSF and IL-4. If required, these may be depleted of CD19⁺ B cells and CD3⁺, CD2⁺ T cells using magnetic beads (Coffin et al.). Culture conditions may include other cytokines such as GM-CSF or IL-4 for the maintenance and, or activity of the dendritic cells or other antigen presenting cells.

T cells and B cells for use in the invention are preferably obtained from cell lines such as lymphoma or leukemia cell lines, T cell hybridomas or B cell hybridomas but may also be isolated from an individual suffering from a disease of the immune system or a recipient for a transplant operation or from a related or unrelated donor individual. T cells and B cells may be obtained from blood or another source (such as lymph nodes, spleen, or bone marrow) and may be enriched or purified by standard procedures. Alternatively whole blood may be used or leukocyte enriched blood or purified white blood cells as a source of T cells and other cell types. It is particularly preferred to use helper T cells (CD4⁺). Alternatively other T cells such as CD8⁺ cells may be used.

Candidate modulators of use in the present invention are brought into contact with a cell of the immune system as described above. In a further step, modulation of Notch signalling by a candidate modulator is detected. Assays for detecting modulation of Notch signalling will be described below. Many of these assays will involve monitoring the expression of a “target gene”.

Assays

Assays for monitoring modulation of Notch signalling are described below.

Any one or more of appropriate targets—such as an amino acid sequence and/or nucleotide sequence—may be used for identifying a compound capable of modulating the Notch signalling pathway in cells of the immune system in any of a variety of drug screening techniques. The target employed in such a test may be free in solution, affixed to a solid support, borne on a cell surface, or located intracellularly. The assay of the present invention is a cell based assay.

The assay may be a screen, whereby a number of agents are tested.

Techniques for drug screening may be based on the method described in Geysen, European Patent No. 0138855, published on Sep. 13, 1984. In summary, large numbers of different small peptide candidate modulators are synthesized on a solid substrate, such as plastic pins or some other surface. The peptide test compounds are reacted with a suitable target or fragment thereof and washed. Bound entities are then detected—such as by appropriately adapting methods well known in the art. A purified target can also be coated directly onto plates for use in drug screening techniques. Plates of use for high throughput screening (HTS) will be multi-well plates, preferably having 96, 384 or over 384 wells/plate. Cells can also be spread as “lawns”. Alternatively, non-neutralising antibodies can be used to capture the peptide and immobilise it on a solid support. High throughput screening, as described above for synthetic compounds, can also be used for identifying organic candidate modulators.

This invention also contemplates the use of competitive drug screening assays in which neutralising antibodies capable of binding a target specifically compete with a test compound for binding to a target.

Various nucleic acid assays are also known. Any conventional technique which is known or which is subsequently disclosed may be employed. Examples of suitable nucleic acid assay are mentioned below and include amplification, PCR, RT-PCR, RNase protection, blotting, spectrometry, reporter gene assays, gene chip arrays and other hybridization methods.

Target gene presence, amplification and/or expression may be measured in a sample directly, for example, by conventional Southern blotting, Northern blotting to quantitate the transcription of target mRNA, dot blotting (DNA or RNA analysis), or in situ hybridisation, using an appropriately labelled probe. Those skilled in the art will readily envisage how these methods may be modified, if desired.

Generation of nucleic acids for analysis from samples generally requires nucleic acid amplification. Many amplification methods rely on an enzymatic chain reaction (such as a polymerase chain reaction, a ligase chain reaction, or a self-sustained sequence replication) or from the replication of all or part of the vector into which it has been cloned. Preferably, the amplification according to the invention is an exponential amplification, as exhibited by for example the polymerase chain reaction.

Many target and signal amplification methods have been described in the literature, for example, general reviews of these methods in Landegren, U., et al., Science 242:229-237 (1988) and Lewis, R., Genetic Engineering News 10:1, 54-55 (1990). These amplification methods may be used in the methods of our invention, and include polymerase chain reaction (PCR), PCR in situ, ligase amplification reaction (LAR), ligase hybridisation, Qbeta bacteriophage replicase, transcription-based amplification system (TAS), genomic amplification with transcript sequencing (GAWTS), nucleic acid sequence-based amplification (NASBA) and in situ hybridisation. Primers suitable for use in various amplification techniques can be prepared according to methods known in the art.

PCR is a nucleic acid amplification method described inter alia in U.S. Pat. Nos. 4,683,195 and 4,683,202. PCR consists of repeated cycles of DNA polymerase generated primer extension reactions. PCR was originally developed as a means of amplifying DNA from an impure sample. The technique is based on a temperature cycle which repeatedly heats and cools the reaction solution allowing primers to anneal to target sequences and extension of those primers for the formation of duplicate daughter strands. RT-PCR uses an RNA template for generation of a first strand cDNA with a reverse transcriptase. The cDNA is then amplified according to standard PCR protocol. Repeated cycles of synthesis and denaturation result in an exponential increase in the number of copies of the target DNA produced. However, as reaction components become limiting, the rate of amplification decreases until a plateau is reached and there is little or no net increase in PCR product. The higher the starting copy number of the nucleic acid target, the sooner this “end-point” is reached. PCR can be used to amplify any known nucleic acid in a diagnostic context (Mok et al., (1994), Gynaecologic Oncology, 52: 247-252).

Self-sustained sequence replication (3SR) is a variation of TAS, which involves the isothermal amplification of a nucleic acid template via sequential rounds of reverse transcriptase (RT), polymerase and nuclease activities that are mediated by an enzyme cocktail and appropriate oligonucleotide primers (Guatelli et al. (1990) Proc. Natl. Acad. Sci. USA 87:1874). Enzymatic degradation of the RNA of the RNA/DNA heteroduplex is used instead of heat denaturation. RNase H and all other enzymes are added to the reaction and all steps occur at the same temperature and without further reagent additions. Following this process, amplifications of 10⁶ to 10⁹ have been achieved in one hour at 42° C.

Ligation amplification reaction or ligation amplification system uses DNA ligase and four oligonucleotides, two per target strand. This technique is described by Wu, D. Y. and Wallace, R. B. (1989) Genomics 4:560. The oligonucleotides hybridise to adjacent sequences on the target DNA and are joined by the ligase. The reaction is heat denatured and the cycle repeated.

Alternative amplification technology can be exploited in the present invention. For example, rolling circle amplification (Lizardi et al., (1998) Nat Genet 19:225) is an amplification technology available commercially (RCAT™) which is driven by DNA polymerase and can replicate circular oligonucleotide probes with either linear or geometric kinetics under isothermal conditions.

In the presence of two suitably designed primers, a geometric amplification occurs via DNA strand displacement and hyperbranching to generate 10¹² or more copies of each circle in 1 hour.

If a single primer is used, RCAT generates in a few minutes a linear chain of thousands of tandemly linked DNA copies of a target covalently linked to that target.

A further technique, strand displacement amplification (SDA; Walker et al., (1992) PNAS (USA) 80:392) begins with a specifically defined sequence unique to a specific target. But unlike other techniques which rely on thermal cycling, SDA is an isothermal process that utilises a series of primers, DNA polymerase and a restriction enzyme to exponentially amplify the unique nucleic acid sequence.

SDA comprises both a target generation phase and an exponential amplification phase.

In target generation, double-stranded DNA is heat denatured creating two single-stranded copies. A series of specially manufactured primers combine with DNA polymerase (amplification primers for copying the base sequence and bumper primers for displacing the newly created strands) to form altered targets capable of exponential amplification.

The exponential amplification process begins with altered targets (single-stranded partial DNA strands with restricted enzyme recognition sites) from the target generation phase.

An amplification primer is bound to each strand at its complementary DNA sequence. DNA polymerase then uses the primer to identify a location to extend the primer from its 3′ end, using the altered target as a template for adding individual nucleotides. The extended primer thus forms a double-stranded DNA segment containing a complete restriction enzyme recognition site at each end.

A restriction enzyme is then bound to the double stranded DNA segment at its recognition site. The restriction enzyme dissociates from the recognition site after having cleaved only one strand of the double-sided segment, forming a nick. DNA polymerase recognises the nick and extends the strand from the site, displacing the previously created strand. The recognition site is thus repeatedly nicked and restored by the restriction enzyme and DNA polymerase with continuous displacement of DNA strands containing the target segment.

Each displaced strand is then available to anneal with amplification primers as above. The process continues with repeated nicking, extension and displacement of new DNA strands, resulting in exponential amplification of the original DNA target.

In an alternative embodiment, the present invention provides for the detection of gene expression at the RNA level. Typical assay formats utilising ribonucleic acid hybridisation include nuclear run-on assays, RT-PCR and RNase protection assays (Melton et al., Nuc. Acids Res. 12:7035. Methods for detection which can be employed include radioactive labels, enzyme labels, chemiluminescent labels, fluorescent labels and other suitable labels.

Real-time PCR uses probes labeled with a fluorescent tag or fluorescent dyes and differs from end-point PCR for quantitative assays in that it is used to detect PCR products as they accumulate rather than for the measurement of product accumulation after a fixed number of cycles. The reactions are characterized by the point in time during cycling when amplification of a target sequence is first detected through a significant increase in fluorescence.

The ribonuclease protection (RNase protection) assay is an extremely sensitive technique for the quantitation of specific RNAs in solution. The ribonuclease protection assay can be performed on total cellular RNA or poly(A)-selected mRNA as a target. The sensitivity of the ribonuclease protection assay derives from the use of a complementary in vitro transcript probe which is radiolabeled to high specific activity. The probe and target RNA are hybridized in solution, after which the mixture is diluted and treated with ribonuclease (RNase) to degrade all remaining single-stranded RNA. The hybridized portion of the probe will be protected from digestion and can be visualized via electrophoresis of the mixture on a denaturing polyacrylamide gel followed by autoradiography. Since the protected fragments are analyzed by high resolution polyacrylamide gel electrophoresis, the ribonuclease protection assay can be employed to accurately map mRNA features. If the probe is hybridized at a molar excess with respect to the target RNA, then the resulting signal will be directly proportional to the amount of complementary RNA in the sample.

PCR technology as described e.g. in section 14 of Sambrook et al., 1989, requires the use of oligonucleotide probes that will hybridise to target nucleic acid sequences. Strategies for selection of oligonucleotides are described below.

As used herein, a probe is e.g. a single-stranded DNA or RNA that has a sequence of nucleotides that includes between 10 and 50, preferably between 15 and 30 and most preferably at least about 20 contiguous bases that are the same as (or the complement of) an equivalent or greater number of contiguous bases. The nucleic acid sequences selected as probes should be of sufficient length and sufficiently unambiguous so that false positive results are minimised. The nucleotide sequences are usually based on conserved or highly homologous nucleotide sequences or regions of polypeptides. The nucleic acids used as probes may be degenerate at one or more positions.

Preferred regions from which to construct probes include 5′ and/or 3′ coding sequences, sequences predicted to encode ligand binding sites, and the like. For example, either the full-length cDNA clone disclosed herein or fragments thereof can be used as probes. Preferably, nucleic acid probes of the invention are labelled with suitable label means for ready detection upon hybridisation. For example, a suitable label means is a radiolabel. The preferred method of labelling a DNA fragment is by incorporating ³²P dATP with the Klenow fragment of DNA polymerase in a random priming reaction, as is well known in the art. Oligonucleotides are usually end-labelled with ³²P-labelled ATP and polynucleotide kinase. However, other methods (e.g. non-radioactive) may also be used to label the fragment or oligonucleotide, including e.g. enzyme labelling, fluorescent labelling with suitable fluorophores and biotinylation.

Preferred are such sequences, probes which hybridise under high-stringency conditions.

Stringency of hybridisation refers to conditions under which polynucleic acids hybrids are stable. Such conditions are evident to those of ordinary skill in the field. As known to those of skill in the art, the stability of hybrids is reflected in the melting temperature (Tm) of the hybrid which decreases approximately 1 to 1.5° C. with every 1% decrease in sequence homology. In general, the stability of a hybrid is a function of sodium ion concentration and temperature. Typically, the hybridisation reaction is performed under conditions of higher stringency, followed by washes of varying stringency.

As used herein, high stringency refers to conditions that permit hybridisation of only those nucleic acid sequences that form stable hybrids in 1M Na+ at 65-68° C. High stringency conditions can be provided, for example, by hybridisation in an aqueous solution containing 6×SSC, 5× Denhardt's, 1% SDS (sodium dodecyl sulphate), 0.1M Na+ pyrophosphate and 0.1 mg/ml denatured salmon sperm DNA as non specific competitor. Following hybridisation, high stringency washing may be done in several steps, with a final wash (about 30 min) at the hybridisation temperature in 0.2−0.1×SSC, 0.1% SDS.

It is understood that these conditions may be adapted and duplicated using a variety of buffers, e.g. formamide-based buffers, and temperatures. Denhardt's solution and SSC are well known to those of skill in the art as are other suitable hybridisation buffers (see, e.g. Sambrook, et al., eds. (1989) Molecular Cloning: A Laboratory Manual, Cold Spring Harbor Laboratory Press, New York or Ausubel, et al., eds. (1990) Current Protocols in Molecular Biology, John Wiley & Sons, Inc.). Optimal hybridisation conditions have to be determined empirically, as the length and the GC content of the hybridising pair also play a role.

Gene expression may also be detected using a reporter system. Such a reporter system may comprise a readily identifiable marker under the control of an expression system, e.g. of the gene being monitored. Fluorescent markers, which can be detected and sorted by FACS, are preferred. Especially preferred are GFP and luciferase. Another type of preferred reporter is cell surface markers, i.e. proteins expressed on the cell surface and therefor easily identifiable. Thus, cell-based screening assays can be designed by constructing cell lines in which the expression of a reporter protein, i.e. an easily assayable protein, such as β-galactosidase, chioramphenicol acetyltransferase (CAT) or luciferase, is dependent on the activation of a Notch. For example, a reporter gene encoding one of the above polypeptides may be placed under the control of a response element which is specifically activated by Notch signalling. Alternative assay formats include assays which directly assess responses in a biological system. If a cell-based assay system is employed, the test compound(s) indentified may then be subjected to in vivo testing to determine their effect on Notch signalling pathway.

In general, reporter constructs useful for detecting Notch signalling by expression of a reporter gene may be constructed according to the general teaching of Sambrook et al. (1989). Typically, constructs according to the invention comprise a promoter of the gene of interest (i.e. of an endogenous target gene), and a coding sequence encoding the desired reporter constructs, for example of GFP or luciferase. Vectors encoding GFP and luciferase are known in the art and available commercially.

Sorting of cells, based upon detection of expression of target genes, may be performed by any technique known in the art, as exemplified above. For example, cells may be sorted by flow cytometry or FACS. For a general reference, see Flow Cytometry and Cell Sorting: A Laboratory Manual (1992) A. Radbruch (Ed.), Springer Laboratory, New York.

Flow cytometry is a powerful method for studying and purifying cells. It has found wide application, particularly in immunology and cell biology: however, the capabilities of the FACS can be applied in many other fields of biology. The acronym F.A.C.S. stands for Fluorescence Activated Cell Sorting, and is used interchangeably with “flow cytometry”. The principle of FACS is that individual cells, held in a thin stream of fluid, are passed through one or more laser beams, causing light to be scattered and fluorescent dyes to emit light at various frequencies. Photomultiplier tubes (PMT) convert light to electrical signals, which are interpreted by software to generate data about the cells. Sub-populations of cells with defined characteristics can be identified and automatically sorted from the suspension at very high purity (˜100%).

FACS can be used to measure target gene expression in cells transfected with recombinant DNA encoding polypeptides. This can be achieved directly, by labelling of the protein product, or indirectly by using a reporter gene in the construct. Examples of reporter genes are β-galactosidase and Green Fluorescent Protein (GFP). β-galactosidase activity can be detected by FACS using fluorogenic substrates such as fluorescein digalactoside (FDG). FDG is introduced into cells by hypotonic shock, and is cleaved by the enzyme to generate a fluorescent product, which is trapped within the cell. One enzyme can therefor generate a large amount of fluorescent product. Cells expressing GFP constructs will fluoresce without the addition of a substrate. Mutants of GFP are available which have different excitation frequencies, but which emit fluorescence in the same channel. In a two-laser FACS machine, it is possible to distinguish cells which are excited by the different lasers and therefor assay two transfections at the same time.

Alternative means of cell sorting may also be employed. For example, the invention comprises the use of nucleic acid probes complementary to mRNA. Such probes can be used to identify cells expressing polypeptides individually, such that they may subsequently be sorted either manually, or using FACS sorting. Nucleic acid probes complementary to mRNA may be prepared according to the teaching set forth above, using the general procedures as described by Sambrook et al. (1989).

In a preferred embodiment, the invention comprises the use of an antisense nucleic acid molecule, complementary to a target mRNA, conjugated to a fluorophore which may be used in FACS cell sorting.

Methods have also been described for obtaining information about gene expression and identity using so-called gene chip arrays or high density DNA arrays (Chee). These high density arrays are particularly useful for diagnostic and prognostic purposes. Use may also be made of In vivo Expression Technology (IVET) (Camilli). IVET identifies target genes up-regulated during say treatment or disease when compared to laboratory culture.

The present invention also provides a method of detection of polypeptides. The advantage of using a protein assay is that Notch activation can be directly measured. Assay techniques that can be used to determine levels of a polypeptide are well known to those skilled in the art. Such assay methods include radioimmunoassays, competitive-binding assays, protein gel assay, Western Blot analysis, antibody sandwich assays, antibody detection, FACS and ELISA assays. For example, polypeptides can be detected by differential mobility on protein gels, or by other size analysis techniques, such as mass spectrometry. The detection means may be sequence-specific. For example, polypeptide or RNA molecules can be developed which specifically recognise polypeptides in vivo or in vitro.

For example, RNA aptamers can be produced by SELEX. SELEX is a method for the in vitro evolution of nucleic acid molecules with highly specific binding to target molecules. It is described, for example, in U.S. Pat. Nos. 5,654,151, 5,503,978, 5,567,588 and 5,270,163, as well as PCT publication WO 96/38579

The invention, in certain embodiments, includes antibodies specifically recognising and binding to polypeptides.

Antibodies may be recovered from the serum of immunised animals. Monoclonal antibodies may be prepared from cells from immunised animals in the conventional manner.

The antibodies of the invention are useful for identifying cells expressing the genes being monitored.

Antibodies according to the invention may be whole antibodies of natural classes, such as IgE and IgM antibodies, but are preferably IgG antibodies. Moreover, the invention includes antibody fragments, such as Fab, F(ab′)2, Fv and ScFv. Small fragments, such Fv and ScFv, possess advantageous properties for diagnostic and therapeutic applications on account of their small size and consequent superior tissue distribution.

The antibodies may comprise a label. Especially preferred are labels which allow the imaging of the antibody in neural cells in vivo. Such labels may be radioactive labels or radioopaque labels, such as metal particles, which are readily visualisable within tissues. Moreover, they may be fluorescent labels or other labels which are visualisable in tissues and which may be used for cell sorting.

In more detail, antibodies as used herein can be altered antibodies comprising an effector protein such as a label. Especially preferred are labels which allow the imaging of the distribution of the antibody in vivo. Such labels can be radioactive labels or radioopaque labels, such as metal particles, which are readily visualisable within the body of a patient. Moreover, they can be fluorescent labels or other labels which are visualisable on tissue

Antibodies as described herein can be produced in cell culture. Recombinant DNA technology can be used to produce the antibodies according to established procedure, in bacterial or preferably mammalian cell culture. The selected cell culture system optionally secretes the antibody product, although antibody products can be isolated from non-secreting cells.

Multiplication of hybridoma cells or mammalian host cells in vitro is carried out in suitable culture media, which are the customary standard culture media, for example Dulbecco's Modified Eagle Medium (DMEM) or RPMI 1640 medium, optionally replenished by a mammalian serum, e.g. foetal calf serum, or trace elements and growth sustaining supplements, e.g. feeder cells such as normal mouse peritoneal exudate cells, spleen cells, bone marrow macrophages, 2-aminoethanol, insulin, transferrin, low density lipoprotein, oleic acid, or the like. Multiplication of host cells which are bacterial cells or yeast cells is likewise carried out in suitable culture media known in the art, for example for bacteria in medium LB, NZCYM, NZYM, NZM, Terrific Broth, SOB, SOC, 2×YT, or M9 Minimal Medium, and for yeast in medium YPD, YEPD, Minimal Medium, or Complete Minimal Dropout Medium.

In vitro production provides relatively pure antibody preparations and allows scale-up to give large amounts of the desired antibodies. Techniques for bacterial cell, yeast or mammalian cell cultivation are known in the art and include homogeneous suspension culture, e.g. in an airlift reactor or in a continuous stirrer reactor, or immobilised or entrapped cell culture, e.g. in hollow fibres, microcapsules, on agarose microbeads or ceramic cartridges.

Large quantities of the desired antibodies can also be obtained by multiplying mammalian cells in vivo. For this purpose, hybridoma cells producing the desired antibodies are injected into histocompatible mammals to cause growth of antibody-producing tumours. Optionally, the animals are primed with a hydrocarbon, especially mineral oils such as pristane (tetramethyl-pentadecane), prior to the injection. After one to three weeks, the antibodies are isolated from the body fluids of those mammals. For example, hybridoma cells obtained by fusion of suitable myeloma cells with antibody-producing spleen cells from Balb/c mice, or transfected cells derived from hybridoma cell line Sp2/0 that produce the desired antibodies are injected intraperitoneally into Balb/c mice optionally pre-treated with pristane, and, after one to two weeks, ascitic fluid is taken from the animals.

The foregoing, and other, techniques are discussed in, for example, Kohler and Milstein, (1975) Nature 256:495-497; U.S. Pat. No. 4,376,110; Harlow and Lane, Antibodies: a Laboratory Manual, (1988) Cold Spring Harbor, incorporated herein by reference. Techniques for the preparation of recombinant antibody molecules is described in the above references and also in, for example, EP 0623679; EP 0368684 and EP 0436597, which are incorporated herein by reference.

The cell culture supernatants are screened for the desired antibodies, preferentially by an enzyme immunoassay, e.g. a sandwich assay or a dot-assay, or a radioimmunoassay.

For isolation of the antibodies, the immunoglobulins in the culture supernatants or in the ascitic fluid can be concentrated, e.g. by precipitation with ammonium sulphate, dialysis against hygroscopic material such as polyethylene glycol, filtration through selective membranes, or the like. If necessary and/or desired, the antibodies are purified by the customary chromatography methods, for example gel filtration, ion-exchange chromatography, chromatography over DEAE-cellulose and/or (immuno-) affinity chromatography, e.g. affinity chromatography with the target antigen, or with Protein-A.

The antibody is preferably provided together with means for detecting the antibody, which can be enzymatic, fluorescent, radioisotopic or other means. The antibody and the detection means can be provided for simultaneous, simultaneous separate or sequential use, in a kit.

The antibodies useful for the invention are assayed for immunospecific binding by any method known in the art. The immunoassays which can be used include but are not limited to competitive and non-competitive assay systems using techniques such as western blots, radioimmunoassays, ELISA, sandwich immunoassays, immunoprecipitation assays, precipitin reactions, gel diffusion precipitin reactions, immunodiffusion assays, agglutination assays, complement-fixation assays, immunoradiometric assays, fluorescent immunoassays and protein A immunoassays. Such assays are routine in the art (see, for example, Ausubel et al., eds, 1994, Current Protocols in Molecular Biology, Vol. 1, John Wiley & Sons, Inc., New York, which is incorporated by reference herein in its entirety). Exemplary immunoassays are described briefly below.

Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding the antibody of interest to the cell lysate, incubating for a period of time (e.g., 1-4 hours) at 4° C., adding protein A and/or protein G sepharose beads to the cell lysate, incubating for about an hour or more at 4° C., washing the beads in lysis buffer and resuspending the beads in SDS/sample buffer. The ability of the antibody of interest to immunoprecipitate a particular antigen can be assessed by, e.g., western blot analysis.

Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, blocking the membrane in blocking solution (e.g., PBS with 3% BSA or non-fat milk), washing the membrane in washing buffer (e.g., PBS-Tween 20), exposing the membrane to a primary antibody (the antibody of interest) diluted in blocking buffer, washing the membrane in washing buffer, exposing the membrane to a secondary antibody (which recognises the primary antibody, e.g., an antihuman antibody) conjugated to an enzymatic substrate (e.g., horseradish peroxidase or alkaline phosphatase) or radioactive molecule (e.g., ³²P or ¹²⁵I diluted in blocking buffer, washing the membrane in wash buffer, and detecting the presence of the antigen.

ELISAs generally comprise preparing antigen, coating the well of a 96 well microtitre plate with the antigen, adding the antibody of interest conjugated to a detectable compound such as an enzymatic substrate (e.g., horseradish peroxidase or alkaline phosphatase) to the well and incubating for a period of time, and detecting the presence of the antigen. In ELISAs the antibody of interest does not have to be conjugated to a detectable compound; instead, a second antibody (which recognises the antibody of interest) conjugated to a detectable compound can be added to the well. Further, instead of coating the well with the antigen, the antibody can be coated to the well. In this case, a second antibody conjugated to a detectable compound can be added following the addition of the antigen of interest to the coated well.

It is convenient when running assays to immobilise one of more of the reactants, particularly when the reactant is soluble. In the present case it may be convenient to immobilse any one of more of the candidate modulator, Notch ligand, immune cell activator or immune cell costimulus. Immobilisation approaches include covalent immobilsation, such as using amine coupling, surface thiol coupling, ligand thiol coupling and aldehyde coupling, and high affinity capture which relies on high affinity binding of a ligand to an immobilsed capturing molecule. Example of capturing molecules include: streptavidin, anti-mouse Ig antibodies, ligand-specific antibodies, protian A, protein G and Tag-specific capture. In one embodiment, immobilisation is achieved through binding to a support, particularly a particulate support which is preferably in the form of a bead.

For assays involving monitoring or detection of tolerised T-cells for use in clinical applications, the assay will generally involve removal of a sample from a patient prior to the step of detecting a signal resulting from cleavage of the intracellular domain.

As used herein, the term “sample” refers to a collection of inorganic, organic or biochemical molecules which is either found in nature (e.g., in a biological- or other specimen) or in an artificially-constructed grouping, such as agents which may be found and/or mixed in a laboratory. The biological sample may refer to a whole organism, but more usually to a subset of its tissues, cells or component parts (e.g. body fluids, including but not limited to blood, mucus, saliva and urine).

The present invention provides a method of detecting novel modulators of Notch signalling. The modulators identified may be used as therapeutic agents—i.e. in therapy applications.

TH2 Modulation

The humoral/TH2 branch of the immune system is generally directed at protecting against extracellular immunogens such as bacteria and parasites through the production of antibodies by B cells; whereas the cellular/TH1 branch is generally directed at intracellular immunogens such as viruses and cancers through the activity of natural killer cells, cytotoxic T lymphocytes and activated macrophages (U.S. Pat. No. 6,039,969). TH2 cells are believed to produce cytokines which stimulate production of IgE antibodies, as well as to be involved with recruitment, proliferation, differentiation, maintenance and survival of eosinophils, which can result in eosinophilia. Eosinophilia is a hallmark of many TH2 mediated diseases, such as asthma, allergy, and atopic dermatitis.

Some diseases that are thought to be caused/mediated in substantial part by TH2 immune response, IL-4/IL-5 cytokine induction, and/or eosinophilia include asthma, allergic rhinitis, systemic lupus erythematosis, Ommen's syndrome (hypereosinophilia syndrome), certain parasitic infections, for example, cutaneous and systemic leishmaniasis, toxoplasma infection and trypanosome infection, and certain fungal infections, for example candidiasis and histoplasmosis, and certain intracellular bacterial infections, such as leprosy and tuberculosis. Additionally, it should also be noted that diseases having a viral or cancer related basis, but with a significant TH2 mediated pathology can also be beneficially treated according to the present invention.

Recent evidence indicates that the immune system can be broken down into two major arms, the humoral and cellular arms. The humoral arm is important in eliminating extracellular pathogens such as bacteria and parasites through production of antibodies by B cells. On the other hand, the cellular arm is important in the elimination of intracellular pathogens such as viruses through the activity of natural killer cells, cytotoxic T lymphocytes and activated macrophages. In recent years it has become apparent that these two arms are activated through distinct T helper cell (TH) populations and their distinct cytokine production profiles. T helper type 1 (TH1) cells are believed to enhance the cellular arm of the immune response and produce predominately the cytokines IL-2 and IFN-.gamma.; whereas, T helper 2 (TH2) cells are believed to enhance the humoral arm of the immune response and produce cytokines, such as interleukin-3 (IL-3), interleukin-4 (IL-4), interleukin-5 (IL-5) and granulocyte-macrophage colony-stimulating factor (GM-CSF). In the TH2 case, IL-3, IL-5 and GM-CSF are thought to stimulate eosinophilopoiesis. In addition, IL-5 facilitates terminal differentiation and cell proliferation of eosinophils and promotes survival, viability and migration of eosinophils, while IL-4 stimulates production of antibodies of the IgE class. IgE is an important component in allergies and asthma. IL-5 may also prime eosinophils for the subsequent actions of other mediators.

In contrast, the TH1 cytokines, IL-2 and IFN gamma, are important in activating macrophages, NK cells and CTL (cytotoxic T lymphocytes). IFN gamma also stimulates B cells to secrete specifically cytophilic antibody for the elimination of virally-infected cells. Interestingly, IFN alpha a macrophage-derived cytokine has been shown to antagonize TH2-type responses. IFN alpha also appears to inhibit the proliferation and cytokine production of TH2 cells and enhances IFN gamma production by TH1 cells. In addition, IFN alpha also appears to inhibit IgE production and antigen-induced increases in IL4 mRNA levels.

One common feature of many TH2 mediated diseases is an accumulation of eosinophils, referred to as eosinophilia. For example, chronic pulmonary inflammation involving eosinophil infiltration is a characteristic hallmark feature of bronchial asthma. Increased numbers of eosinophils have been observed in blood, bronchoalveolar lavage fluid and pulmonary tissue in patients with asthma, but the mechanism(s) responsible for their recruitment into and regulation within pulmonary tissues undergoing allergic or pro-inflammatory reactions has not been fully understood. Mediators and cytokines from T-lymphocytes and effector cells such as basophils, mast cells, macrophages and eosinophils have been implicated in enhancing cell maturation, chemotaxis and activation of eosinophils. Evidence suggests that an association exists between the immune system, especially CD4+ T cells, and eosinophils and eosinophil recruitment. Studies in asthmatics and in animal models of allergic pulmonary responses support this notion with the evidence of close correlations between the relative numbers of T cells and activated eosinophils in the airways.

Examples of diseases which may be treated by reducing a TH2 response according to the present invention include include asthma, allergy, atopic dermatitis, early HIV disease, infectious mononucleosis, systemic lupus erythematosis, parasitic infections, for example, cutaneous and systemic leishmaniasis, Toxoplasma infection and Trypanosome infection, certain fungal infections, for example Candidiasis and Histoplasmosis, and intracellular bacterial infections, such as leprosy and tuberculosis.

TH1 Mediated Disease

Many organ-specific autoimmune conditions appear to involve pathological Th1 response. These data have been reviewed (Modlin and Nutman, Current Opinion in Immunol., 5, pp. 511-17 (1993); Romagnani et al., Ann. Rev. Immunol., 12, pp. 227-57 (1994)). These organ-specific autoimmune conditions include: multiple sclerosis, insulin-dependent diabetes, sympathetic ophthalmia, uveitis and psoriasis.

Insulin-dependent diabetes mellitus is an autoimmune disease in which the insulin-producing beta pancreatic cells are destroyed by leukocytes infiltrating into the islets of Langerhans. Diabetes can be rapidly induced in neonatal nonobese diabetic (NOD) mice by transferring activated prediabetic splenocytes. Recently, Th1- or Th2-like cells, otherwise genetically similar, were transferred into neonatal NOD mice. Only the Th1 cells rapidly induced diabetes—and in almost all recipients (Katz et al., Science, 268, pp. 1185-88 (1995)).

Several systemic autoimmune diseases, including various arthritides, are Th1 cell-associated. Rheumatoid arthritis and Sjorgren's syndrome both appear to involve Th0 and Th1 cells. In contrast, systemic lupus erythematosus (SLE) appears to have an aberrant Th0/Th2 dominated response.

Some chronic inflammatory diseases also appear to have an aberrant Th1 type response, including inflammatory bowel disease, sarcoidosis of the lung and allograft rejection. Inflammatory bowel disease (IBD) in humans encompasses at least two categories, ulcerative colitis and Crohn's disease. Both disorders are believed to result from immunopathologic autoimmune like disorders. In some mouse models of IBD, it is clear that some agents that block Th1 responses can block the development or course of the disease (F. Powrie et al., Immunity 1:553 1994). It is possible that inhibition of the Th1 component of the immune response would have beneficial effects in human IBD. Many models of IBD have been described and have been reviewed (C. Elson et al., Gastroenterology 109:1344 1995). There are at least three groups of models, chemically induced, polymer/microbial-induced and immunological types using mutant mice.

In one commonly used polymer/microbial-induced model, dextran sulphate solution is introduced into the drinking water of mice and upon ingestion, the epithelial lining of the gut is irritated leading to a profound immune response to the damage. The animals develop colitis which is manifested as diarrhea, blood in the stool, loss of body weight and a shortening of the colon length due to expansion of the colon wall. This model induces a left-sided colitis and epithelial dysplasia which can lead to cancer which are features of ulcerative colitis.

A second model consists of transplanting a selected set of CD4 T cells into a scid mouse, i.e. a mouse lacking T and B cells (F. Powrie et al. International Immunology 5:1461-1471 1993; Morrissey et al., J. Exp. Med. 178:237 1993). As the selected cells, called CD45RB.sup.hi cells expand and reconstitute the scid mouse, the normal mechanisms preventing the appearance of autoreactive T cells are dysfunctional and autoreactive cells develop. In rats, cells reactive with many organs are observed whereas, in the mouse, the reactivity occurs primarily in the bowel. Agents which either alter the way the autoreactive cells expand and develop or agents which can block the ability of the cells to attack the bowel will have efficacy in this model. Moreover, as this model at least partially mimics the pathological development of autoreactive immune system cells, treatments that block this model may actually have disease modifying behavior in humans. In this model, antibodies to TNF can block disease (F. Powrie et al. Immunity 1, 552 1994) and these antibodies have been found to be efficacious in the treatment of human disease (H. M. van Dullemen et al. Gastroenterology 109:109 1995). Thus, this model can forecast which agents may be therapeutically useful in IBD.

In general, the exact contribution of auto-antibodies versus specific T cells has not been delineated in these autoimmune diseases. Cellular responses may make major contributions to pathogenicity in those systemic autoimmune diseases currently thought to be primarily antibody driven, e.g. the various arthritides.

The normal immune response to some pathogenic infectious agents also elicits a Th1 response that can become excessive and present itself as a medical problem. Examples of granulomatous reactions (a class of DTH response described above) that lead to severe medical problems include leprosy, granuloma formation in the lungs of tuberculosis patients, sarcoidosis and schistosomiasis (Roitt et al., Immunology, pp. 22.5-6 (Mosby-Year Book Europe Ltd., 3d ed. 1993). Psoriasis is also likely to be mediated by Th1 cells.

Cytolytic T cells, i.e. CTLs (CD8 positive T cells) may also subdivide into Th1- and Th2-like populations. Therefore it is possible that much of what is known regarding the Th groups will also apply to CD8+ cells, which are primarily involved in anti-viral and grafted tissue rejection responses.

The ability to selectively suppress Th1 (or indirectly stimulate Th2) cell responses is useful for treating abnormalities in diverse cell-mediated immune responses including various autoimmune and chronic inflammatory conditions, antigen tolerance, and cellular rejection of tissue grafts and organ transplants.

As discussed above, treatment of Th1 cell-based immunological conditions generally employs immunomodulatory and immunosuppressive agents which have pleiotropic effects on a wide variety of cell types and immunological responses. These non-specific immunosuppressive agents are generally required in high and often cytotoxic doses that cause adverse side effects.

The ability to shift the character of an immunological response is supported in the recent study of mouse diabetes discussed above (Katz et al., Science, 268, pp. 1185-88 (1995)), and in an allogeneic transplant model (Sayegh et al., J. Exp. Med., 181, pp. 1869-74 (1995)). In the latter study, a fusion protein that blocks the CD28-B7 T cell costimulatory pathway was shown to induce renal graft tolerance. The tolerance correlated with a decrease in Th1 cytokines and an increase in Th2 cytokines in vivo.

Some Th1-associated reactions are critical components of a number of cell-mediated immune responses (Romagnani, S., Ann. Rev. Immunol., 12, pp. 227-57 (1994)), and absolute inhibition of Th1 cell activity may not be desirable in certain circumstances. For example, a mouse can effectively resist a parasitic infection when a good Th1 response can be mounted. Infectious agents such as Listeria and Toxoplasma also elicit strong Th1-type responses. In humans, mycobacterium tuberculosis responses appear to be Th1-based. Leishmaniasis pathogenicity correlates with responses similar to the Th1 responses characterized in mouse (Reed and Scott, Current Opinion in Immunol., 5, pp. 524-31 (1993)).

Therapy

The term “therapy” includes curative effects, alleviation effects, and prophylactic effects. The therapy may be on humans or animals.

Modulators identified by the assay method of the present invention may be used to treat disorders and/or conditions of the immune system. In particular, the compounds can be used in the treatment of T cell mediated diseases or disorders. A detailed description of the conditions affected by the Notch signalling pathway may be found in our WO98/20142, WO00/36089 and WO/00135990.

Diseased or infectious states that may be described as being mediated by T cells include, but are not limited to, any one or more of asthma, allergy, tumour induced aberrations to the T cell system and infectious diseases such as those caused by Plasmodium species, Microfilariae, Helminths, Mycobacteria, HIV, Cytomegalovirus, Pseudomonas, Toxoplasma, Echinococcus, Haemophilus influenza type B, measles, Hepatitis C or Toxicara. Thus particular conditions that may be treated or prevented which are mediated by T cells include multiple sclerosis, rheumatoid arthritis and diabetes. The present invention may also be used in organ transplantation or bone marrow transplantation. The present invention is also useful in treating immune disorders such as autoimmune disorders or graft rejection such as allograft rejection.

Examples of autoimmune disorders range from organ specific diseases (such as thyroiditis, insulitis, multiple sclerosis, iridocyclitis, uveitis, orchitis, hepatitis, Addison's disease, myasthenia gravis) to systemic illnesses such as rheumatoid arthritis or lupus erythematosus. Other disorders include immune hyperreactivity, such as allergic reactions.

In more detail, organ-specific autoimmune diseases include multiple sclerosis, insulin dependent diabetes mellitus, several forms of anemia (aplastic, hemolytic), autoimmune hepatitis, thyroiditis, insulitis, iridocyclitis, skleritis, uveitis, orchitis, myasthenia gravis, idiopathic thrombocytopenic purpura, inflammatory bowel diseases (Crohn's disease, ulcerative colitis).

Systemic autoimmune diseases include: rheumatoid arthritis, juvenile arthritis, scleroderma and systemic sclerosis, sjogren's syndrom, undifferentiated connective tissue syndrome, antiphospholipid syndrome, different forms of vasculitis (polyarteritis nodosa, allergic granulomatosis and angiitis, Wegner's granulomatosis, Kawasaki disease, hypersensitivity vasculitis, Henoch-Schoenlein purpura, Behcet's Syndrome, Takayasu arteritis, Giant cell arteritis, Thrombangiitis obliterans), lupus erythematosus, polymyalgia rheumatica, essentiell (mixed) cryoglobulinemia, Psoriasis vulgaris and psoriatic arthritis, diffus fasciitis with or without eosinophilia, polymyositis and other idiopathic inflammatory myopathies, relapsing panniculitis, relapsing polychondritis, lymphomatoid granulomatosis, erythema nodosum, ankylosing spondylitis, Reiter's syndrome, different forms of inflammatory dermatitis.

A more extensive list of disorders includes: unwanted immune reactions and inflammation including arthritis, including rheumatoid arthritis, inflammation associated with hypersensitivity, allergic reactions, asthma, systemic lupus erythematosus, collagen diseases and other autoimmune diseases, inflammation associated with atherosclerosis, arteriosclerosis, atherosclerotic heart disease, reperfusion injury, cardiac arrest, myocardial infarction, vascular inflammatory disorders, respiratory distress syndrome or other cardiopulmonary diseases, inflammation associated with peptic ulcer, ulcerative colitis and other diseases of the gastrointestinal tract, hepatic fibrosis, liver cirrhosis or other hepatic diseases, thyroiditis or other glandular diseases, glomerulonephritis or other renal and urologic diseases, otitis or other oto-rhino-laryngological diseases, dermatitis or other dermal diseases, periodontal diseases or other dental diseases, orchitis or epididimo-orchitis, infertility, orchidal trauma or other immune-related testicular diseases, placental dysfunction, placental insufficiency, habitual abortion, eclampsia, pre-eclampsia and other immune and/or inflammatory-related gynaecological diseases, posterior uveitis, intermediate uveitis, anterior uveitis, conjunctivitis, chorioretinitis, uveoretinitis, optic neuritis, intraocular inflammation, e.g. retinitis or cystoid macular oedema, sympathetic ophthalmia, scleritis, retinitis pigmentosa, immune and inflammatory components of degenerative fondus disease, inflammatory components of ocular trauma, ocular inflammation caused by infection, proliferative vitreo-retinopathies, acute ischaemic optic neuropathy, excessive scarring, e.g. following glaucoma filtration operation, immune and/or inflammation reaction against ocular implants and other immune and inflammatory-related ophthalmic diseases, inflammation associated with autoimmune diseases or conditions or disorders where, both in the central nervous system (CNS) or in any other organ, immune and/or inflammation suppression would be beneficial, Parkinson's disease, complication and/or side effects from treatment of Parkinson's disease, AIDS-related dementia complex HIV-related encephalopathy, Devic's disease, Sydenham chorea, Alzheimer's disease and other degenerative diseases, conditions or disorders of the CNS, inflammatory components of stokes, post-polio syndrome, immune and inflammatory components of psychiatric disorders, myelitis, encephalitis, subacute sclerosing pan-encephalitis, encephalomyelitis, acute neuropathy, subacute neuropathy, chronic neuropathy, Guillaim-Barre syndrome, Sydenham chora, myasthenia gravis, pseudo-tumour cerebri, Down's Syndrome, Huntington's disease, amyotrophic lateral sclerosis, inflammatory components of CNS compression or CNS trauma or infections of the CNS, inflammatory components of muscular atrophies and dystrophies, and immune and inflammatory related diseases, conditions or disorders of the central and peripheral nervous systems, post-traumatic inflammation, septic shock, infectious diseases, inflammatory complications or side effects of surgery or organ, inflammatory and/or immune complications and side effects of gene therapy, e.g. due to infection with a viral carrier, or inflammation associated with AIDS, to suppress or inhibit a humoral and/or cellular immune response, to treat or ameliorate monocyte or leukocyte proliferative diseases, e.g. leukaemia, by reducing the amount of monocytes or lymphocytes, for the prevention and/or treatment of graft rejection in cases of transplantation of natural or artificial cells, tissue and organs such as cornea, bone marrow, organs, lenses, pacemakers, natural or artificial skin tissue.

The present invention is also useful in cancer therapy, particularly in diseases involving the conversion of epithelial cells to cancer. In particular, the invention may be useful in increasing immune response to cancer by modulating production of key cytokines, for example by use of an inhibitor of Notch signalling. The present invention is especially useful in relation to adenocarcinomas such as: small cell lung cancer, and cancer of the kidney, uterus, prostrate, bladder, ovary, colon and breast. Thus, the present application has application in the treatment of malignant and pre-neoplastic disorders. The present invention is especially useful in relation to adenocarcinomas such as: small cell lung cancer, and cancer of the kidney, uterus, prostrate, bladder, ovary, colon and breast. For example, malignancies which may be treatable according to the present invention include acute and chronic leukemias, lymphomas, myelomas, sarcomas such as Fibrosarcoma, myxosarcoma, liposarcoma, lymphangioendotheliosarcoma, angiosarcoma, endotheliosarcoma, chondrosarcoma, osteogenic sarcoma, chordoma, lymphangiosarcoma, synovioma, mesothelioma, leimyosarcoma, rhabdomyosarcoma, colon carcinoma, ovarian cancer, prostate cancer, pancreatic cancer, breasy cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, choriocarcinoma, renal cell carcinoma, hepatoma, bile duct carcinoma seminoma, embryonal carcinoma, cervical cancer, testicular tumour, lung carcinoma, small cell lung carcinoma, bladder carcinoma, epithelial carcinoma, glioma, astrocytoma, ependymoma, pinealoma, hemangioblastoma, acoustic neuoma, medulloblastoma, craniopharyngioma, oligodendroglioma, menangioma, melanoma, neutroblastoma and retinoblastoma.

Pharmaceutical Compositions

The present invention provides a pharmaceutical composition comprising administering a therapeutically effective amount of at least one compound identified by the method of the present invention and a pharmaceutically acceptable carrier, diluent or excipients (including combinations thereof).

The pharmaceutical compositions may be for human or animal usage in human and veterinary medicine and will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s).

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

There may be different composition/formulation requirements dependent on the different delivery systems. By way of example, the pharmaceutical composition of the present invention may be formulated to be delivered using a mini-pump or by a mucosal route, for example, as a nasal spray or aerosol for inhalation or ingestable solution, or parenterally in which the composition is formulated by an injectable form, for delivery, by, for example, an intravenous, intramuscular or subcutaneous route. Alternatively, the formulation may be designed to be delivered by both routes.

Where the compound is to be delivered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the pharmaceutical compositions can be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

Administration

Typically, a physician will determine the actual dosage which will be most suitable for an individual subject and it will vary with the age, weight and response of the particular patient. The dosages below are exemplary of the average case. There can, of course, be individual instances where higher or lower dosage ranges are merited.

The compositions of the present invention may be administered by direct injection. The composition may be formulated for parenteral, mucosal, intramuscular, intravenous, subcutaneous, intraocular or transdermal administration.

The term “administered” includes delivery by viral or non-viral techniques. Viral delivery mechanisms include but are not limited to adenoviral vectors, adeno-associated viral (AAV) vectos, herpes viral vectors, retroviral vectors, lentiviral vectors, and baculoviral vectors. Non-viral delivery mechanisms include lipid mediated transfection, liposomes, immunoliposomes, lipofectin, cationic facial amphiphiles (CFAs) and combinations thereof. The routes for such delivery mechanisms include but are not limited to mucosal, nasal, oral, parenteral, gastrointestinal, topical, or sublingual routes.

The term “administered” includes but is not limited to delivery by a mucosal route, for example, as a nasal spray or aerosol for inhalation or as an ingestable solution; a parenteral route where delivery is by an injectable form, such as, for example, an intravenous, intramuscular, intradermal, intra-articular, intrathecal, intra-peritoneal or subcutaneous route, or via the alimentary tract (for example, via the Peyers patches).

The routes of administration and dosages described are intended only as a guide since a skilled practitioner will be able to determine readily the optimum route of administration and dosage for any particular patient depending on, for example, the age, weight and condition of the patient. Preferably the pharmaceutical compositions are in unit dosage form. The present invention includes both human and veterinary applications.

Preparation of Primed APCs and Lymphocytes

According to one aspect of the invention immune cells may be used to present antigens or allergens and/or may be treated to modulate expression or interaction of Notch, a Notch ligand or the Notch signalling pathway. Thus, for example, Antigen Presenting Cells (APCs) may be cultured in a suitable culture medium such as DMEM or other defined media, optionally in the presence of a serum such as fetal calf serum. Optimum cytokine concentrations may be determined by titration. One or more substances capable of up-regulating or down-regulating the Notch signalling pathway are then typically added to the culture medium together with the antigen of interest. The antigen may be added before, after or at substantially the same time as the substance(s). Cells are typically incubated with the substance(s) and antigen for at least one hour, preferably at least 3 hours, if necessary for at least 12 hours or more at 37° C. If required, a small aliquot of cells may be tested for modulated target gene expression as described above. Alternatively, cell activity may be measured by the inhibition of T cell activation by monitoring surface markers, cytokine secretion or proliferation as described in WO98/20142. APCs transfected with a nucleic acid construct directing the expression of, for example Serrate, may be used as a control.

As discussed above, polypeptide substances may be administered to APCs by introducing nucleic acid constructs/viral vectors encoding the polypeptide into cells under conditions that allow for expression of the polypeptide in the APC. Similarly, nucleic acid constructs encoding antigens may be introduced into the APCs by transfection, viral infection or viral transduction. The resulting APCs that show increased levels of a Notch signalling are now ready for use.

The techniques described below are described in relation to T cells, but are equally applicable to B cells. The techniques employed are essentially identical to that described for APCs alone except that T cells are generally co-cultured with the APCs. However, it may be preferred to prepare primed APCs first and then incubate them with T cells. For example, once the primed APCs have been prepared, they may be pelleted and washed with PBS before being resuspended in fresh culture medium. This has the advantage that if, for example, it is desired to treat the T cells with a different substance(s) capable of modulating Notch to that used with the APC, then the T cell will not be brought into contact with the different substance(s) used in the APC. Alternatively, the T cell may be incubated with a first substance (or set of substances) to modulate Notch signalling, washed, resuspended and then incubated with the primed APC in the absence of both the substance(s) used to modulate the APC and the substance(s) used to modulate the T cell. Alternatively, T cells may be cultured and primed in the absence of APCs by use of APC substitutes such as anti-TCR antibodies (e.g. anti-CD3) with or without antibodies to costimulatory molecules (e.g. anti-CD28) or alternatively T cells may be activated with MHC-peptide complexes (e.g. tetramers).

Incubations will typically be for at least 1 hour, preferably at least 3 or 6 hours, in suitable culture medium at 37° C. Induction of immunotolerance may be determined by subsequently challenging T cells with antigen and measuring IL-2 production compared with control cells not exposed to APCs.

T cells or B cells which have been primed in this way may be used according to the invention to induce immunotolerance in other T cells or B cells.

Antigens and Allergens

In one embodiment, the agents of the present invention may be administered in simultaneous, separate or sequential combination with antigens or antigenic determinants (or polynucleotides coding therefor), to modify (increase or decrease) the immune response to such antigens or antigenic determinants.

An antigen suitable for use in the present invention may be any substance that can be recognised by the immune system, and is generally recognised by an antigen receptor. Preferably the antigen used in the present invention is an immunogen. An allergic response occurs when the host is re-exposed to an antigen that it has encountered previously.

The immune response to antigen is generally either cell mediated (T cell mediated killing) or humoral (antibody production via recognition of whole antigen). The pattern of cytokine production by TH cells involved in an immune response can influence which of these response types predominates: cell mediated immunity (TH1) is characterised by high IL-2 and IFNγ but low IL-4 production, whereas in humoral immunity (TH2) the pattern is low IL-2 and IFNγ but high IL-4, IL-5 and IL-13. Since the secretory pattern is modulated at the level of the secondary lymphoid organ or cells, then pharmacological manipulation of the specific TH cytokine pattern can influence the type and extent of the immune response generated.

The TH1-TH2 balance refers to the relative representation of the two different forms of helper T cells. The two forms have large scale and opposing effects on the immune system. If an immune response favours TH1 cells, then these cells will drive a cellular response, whereas TH2 cells will drive an antibody-dominated response. The type of antibodies responsible for some allergic reactions is induced by TH2 cells.

The antigen or allergen (or antigenic determinant thereof) used in the present invention may be a peptide, polypeptide, carbohydrate, protein, glycoprotein, or more complex material containing multiple antigenic epitopes such as a protein complex, cell-membrane preparation, whole cells (viable or non-viable cells), bacterial cells or virus/viral component. In particular, it is preferred to use antigens known to be associated with autoimmune diseases such as myelin basic protein (associated with multiple sclerosis), collagen (associated with rheumatoid arthritis), and insulin (diabetes), or antigens associated with rejection of non-self tissue such as MHC antigens or antigenic determinants thereof. Where primed the APCs and/or T cells of the present invention are to be used in tissue transplantation procedures, antigens may be obtained from the tissue donor. Polynucleotides coding for antigens or antigenic determinants which may be expessed in a subject may also be used.

The present invention will now further be described with reference to the following non-limiting Examples:

### EXAMPLES

### Example 1

### CD4+ Cell Purification

Spleens were removed from mice (variously Balb/c females, 8-10 weeks, C57B/6 females, 8-10 weeks, CARD 1 females, 8-10 weeks (D011.10 transgenic, CAR transgenic)) and passed through a 0.2 μM cell strainer into 20 ml R10F medium (R10F-RPMI 1640 media (Gibco Cat No 22409) plus 2 mM L-glutamine, 50 μg/ml Penicillin, 50 μg/ml Streptomycin, 5×10⁻⁵ M β-mercapto-ethanol in 10% fetal calf serum). The cell suspension was spun (1150 rpm 5 min) and the media removed.

The cells were incubated for 4 minutes with 5 ml ACK lysis buffer (0.15M NH₄Cl, 1.0M KHCO₃, 0.1 mM Na₂EDTA in double distilled water) per spleen (to lyse red blood cells). The cells were then washed once with R10F medium and counted. CD4+ cells were purified from the suspensions by positive selection on a Magnetic Associated Cell Sorter (MACS) column (Miltenyi Biotec, Bisley, UK: Cat No 130-042-401) using CD4 (L3T4) beads (Miltenyi Biotec Cat No 130-049-201), according to the manufacturer's directions.

### Example 2

### hDelta1-IgG4Fc Fusion Protein

A fusion protein comprising the extracellular domain of human Delta1 fused to the Fc domain of human IgG4 (“hDelta1-IgG4Fc”) was prepared by inserting a nucleotide sequence coding for the extracellular domain of human Delta1 (see, e.g. Genbank Accession No AF003522) into the expression vector pCONγ (Lonza Biologics, Slough, UK) and expressing the resulting construct in CHO cells. The amino acid sequence of the resulting expressed fusion protein was as follows (SEQ ID NO: 27):

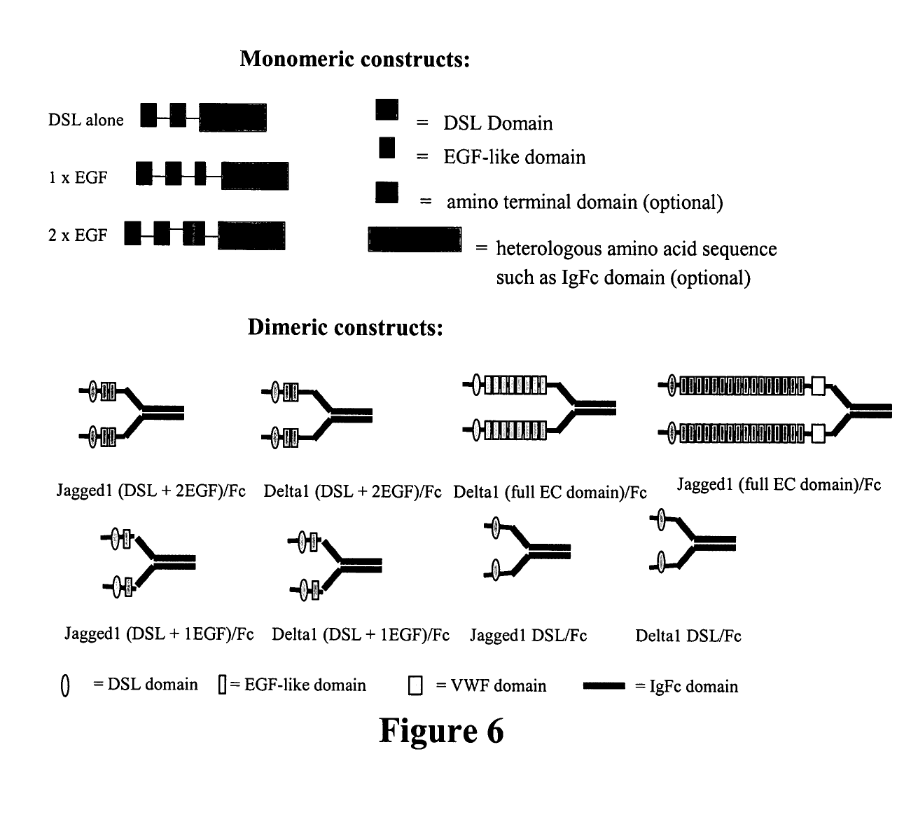

MGSRCALALAVLSALLCQVWSSGVFELKLQEFVNKKGLLGNRNCCRGGAG
PPPCACRTFFRVCLKHYQASVSPEPPCTYGSAVTPVLGVDSFSLPDGGGA
DSAFSNPIRFPFGFTWPGTFSLIIEALHTDSPDDLATENPERLISRLATQ
RHLTVGEEWSQDLHSSGRTDLKYSYRFVCDEHYYGEGCSVFCRPRDDAFG
HFTCGERGEKVCNPGWKGPYCTEPICLPGCDEQHGFCDKPGECKCRVGWQ
GRYCDECIRYPGCLHGTCQQPWQCNCQEGWGGLFCNQDLNYCTHHKPCKN
GATCTNTGQGSYTCSCRPGYTGATCELGIDECDPSPCKNGGSCTDLENSY
SCTCPPGFYGKICELSAMTCADGPCFNGGRCSDSPDGGYSCRCPVGYSGF
NCEKKIDYCSSSPCSNGAKCVDLGDAYLCRCQAGFSGRHCDDNVDDCASS
PCANGGTCRDGVNDFSCTCPPGYTGRNCSAPVSRCEHAPCHNGATCHERG
HGYVCECARGYGGPNCQFLLPELPPGPAVVDLTEKLEASTKGPSVFPLAP
CSRSTSESTAALGCLVKDYFPEPVTVSWNSGALTSGVHTFPAVLQSSGLY
SLSSVVTVPSSSLGTKTYTCNVDHKPSNTKVDKRVESKYGPPCPSCPAPE
FLGGPSVFLFPPKPKDTLMISRTPEVTCVVVDVSQEDPEVQFNWYVDGVE
VHNAKTKPREEQFNSTYRVVSVLTVLHQDWLNGKEYKCKVSNKGLPSSIE
KTISKAKGQPREPQVYTLPPSQEEMTKNQVSLTCLVKGFYPSDIAVEWES
NGQPENNYKTTPPVLDSDGSFFLYSRLTVDKSRWQEGNVFSCSVMHEALH
NHYTQKSLSLSLGK

Wherein the first underlined sequence is the signal peptide (cleaved from the mature protein) and the second underlined sequence is the IgG4 Fc sequence. The protein normally exists as a dimer linked by cysteine disulphide bonds (see e.g. schematic representation in FIG. 6). A corresponding protein was prepared using mouse Delta1.

The following protocols were used for coating 96 well flat-bottomed plates with antibodies.

A) The plates were coated with Dulbecco's Phosphate Buffered Saline (DPBS) plus 1 μg/ml anti-CD3 antibody (Pharmingen, San Diego, US: Cat No 553058, Clone No 145-2C11) plus 1 μg/ml anti-IgG4 antibody (Pharmingen Cat No 555878). 100 μl of coating mixture was used per well. Plates were incubated overnight at 4° C. then washed with DPBS. Each well then received either 100 μl DPBS or 100 μl DPBS plus 10 μg/ml Notch ligand (mouse Delta 1 extracellular domain/Ig4Fc fusion protein; Fc-delta). The plates were incubated for 2-3 hours at 37° C. then washed again with DPBS before cells (prepared as in Example 1) were added.

B) Alternatively, the plates were coated with DPBS plus 1 μg/ml anti-hamsterIgG antibody (Pharmingen Cat No 554007) plus 1 μg/ml anti-IgG4 antibody. 100 μl of coating mixture was added per well. Plates were incubated overnight at 4° C. then washed with DPBS. Each well then received either 100 μl DPBS plus anti-CD3 antibody (1 μg/ml) or, 100 μl DPBS plus anti-CD3 antibody (1 μg/ml) plus Fc-delta (10 μg/ml). The plates were incubated for 2-3 hours at 37° C. then washed again with DPBS before cells (prepared as in Example 1) were added.

### Example 3

### Primary Polyclonal Stimulation

CD4+ cells were cultured in 96 well, flat-bottomed plates pre-coated according to Example 2 (A) or 2 (B). Cells were re-suspended, following counting, at 2×10⁶/ml in R10F medium plus 4 μg/ml anti-CD28 antibody (Pharmingen, Cat No 553294, Clone No 37.51). 100 μl cell suspension was added per well. 100 μl of R10F medium was then added to each well to give a final volume of 200 μl (2×10⁵ cells/well, anti-CD28 final concentration 2 μg/ml). The plates were then incubated at 37° C. for 72 hours.

125 μl supernatant was then removed from each well and stored at −20° C. until tested by ELISA for IL-10, IFNg and IL-13 using antibody pairs from R & D Systems (Abingdon, UK). The cells were then split 1 in 3 into new wells (not coated) and fed with R10F medium plus recombinant human IL-2 (2.5 ng/ml, PeproTech Inc, London, UK: Cat No 200-02).

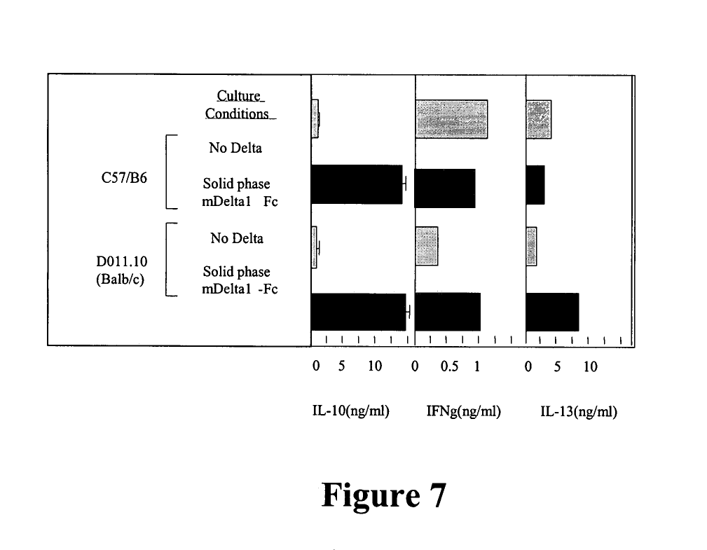

Results are shown in FIG. 7.

### Example 4

### Real Time PCR Analysis of Primary Stimulated CD4+ Cells

Murine (Balb/c) stimulated CD4⁺ T-cells from Example 3 were harvested at 4, 16 and 24 hours. Total cellular RNA was isolated using the RNeasy™ RNA isolation kit (Qiagen, Crawley, UK) according to the manufacturer's guidelines.

In each case 1 μg of total RNA was reverse transcribed using SuperScript™ II Reverse Transcriptase (Invitrogen, Paisley, UK) using Oligo dT₍₁₂₋₁₈₎ or a random decamer mix according to the manufacturer's guidelines. After synthesis, Oligo dT₍₁₂₋₁₈₎- and random decamer-primed cDNAs were mixed in equal proportions to provide the working cDNA sample for real-time quantitative PCR analysis.

Real-time quantitative PCR was performed using the Roche Lightcycler™ system (Roche, UK) and SYBR green detection chemistry according to the manufacturer's guidelines. The following HPLC-purified primer pairs were used for cDNA-specific amplification (5′ to 3′):

| | |
|---|---|
| mouse 18s rRNA: | |
| Forward GTAACCCGTTGAACCCCATT | (SEQ ID NO:28) |
| Reverse CCATCCAATCGGTAGTAGCG | (SEQ ID NO:29) |
| mouse Hes-1: | |
| Forward GGTGCTGATAACAGCGGAAT | (SEQ ID NO:30) |
| Reverse ATTTTTGGAATCCTTCACGC | (SEQ ID NO:31) |

The endpoint used in real-time PCR quantification, the Crossing Point (Cₚ), is defined as the PCR cycle number that crosses an algorithm-defined signal threshold. Quantitative analysis of gene-specific cDNA was achieved firstly by generating a set of standards using the Cₚs from a set of serially-diluted gene-specific amplicons which had been previously cloned into a plasmid vector (pCR2.1, Invitrogen). These serial dilutions fall into a standard curve against which the Cₚs from the cDNA samples were compared. Using this system, expression levels of the 18S rRNA house-keeping gene were generated for each cDNA sample. Hes-1 was then analysed by the same method using serially-diluted Hes-1-specific standards, and the Hes-1 value divided by the 18S rRNA value to generate a value, which represents the relative expression of Hes-1 in each cDNA sample. All Cp analysis was performed using the Second Derivative Maximum algorithm within the Lightcycler system software.

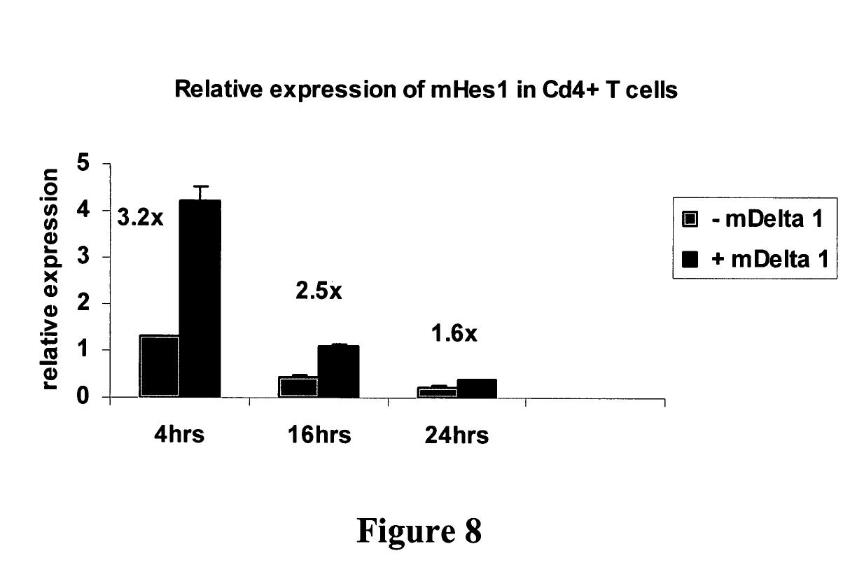

Results (HES-1 expression relative to 18S rRNA expression with and without Fc-delta) are shown in FIG. 8.

### Example 5

### Screening under Polarising Conditions

Plates were coated and CD4+ cells added as in Example 2 (A).

The procedure of Example 3 was then followed, except that instead of adding 100 μl R10F medium per well as in Example 3, 100 μl of polarising cocktail was added per well as follows:

Un-polarised cells: R10F medium.

Th1 polarised cells: R10F medium plus anti-IL-4 antibody (10 μg/ml, Pharmingen Cat No 554432) plus IL-12 (10 ng/ml, Peprotech 210-12).

Th2 polarised cells: R10F medium plus anti-IL-12 antibody (10 μg/ml, Pharmingen Cat No 554475) plus anti-IFNg antibody (1 μg/ml, Pharmingen Cat No 554408) plus IL-4 (10 ng/ml, Peprotech Cat No 214-14).

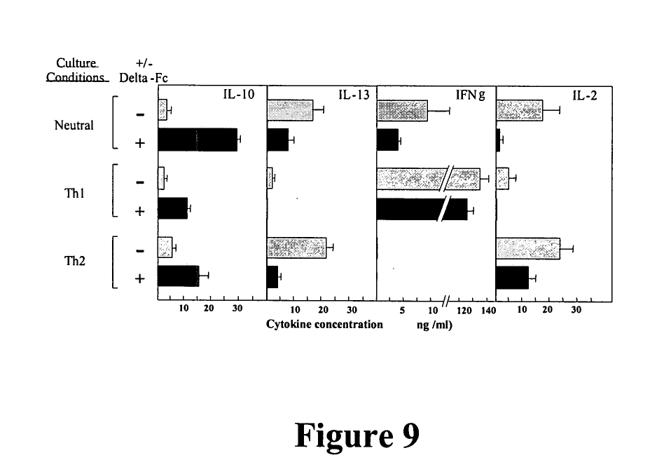

Cells were then stimulated and cytokines (IL-10, IFNγ and IL-13) measured by ELISA as described in Example 3. Results are shown in FIG. 9.

### Example 6

### Soluble Ligand

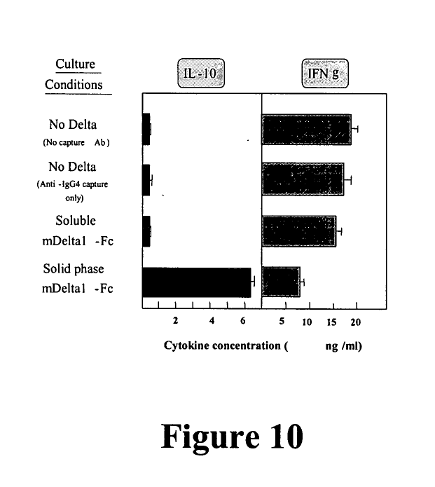

The procedure of Example 2(A) (with the modification that ligand was not added to the plate) and Example 3 (with the modification that soluble Fc-delta was added with the R10F medium) was used to compare soluble Fc-delta with plate-bound Fc-delta against controls. Results are shown in FIG. 10.

### Example 7

Secondary Stimulation

7 days after primary stimulation all cells were harvested and counted then stimulated in one of three ways as follows:

Re-Stimulation

Cells were re-stimulated exactly as for primary stimulation (Example 3).

Re-Challenge on Anti-CD3/CD28

96-well flat-bottomed plates were coated with PBS plus 1 μg/ml anti-CD3 antibody. The plates were incubated overnight at 4° C. then washed with DPBS.

The cells were re-suspended at 2×10⁶/ml in R10F medium plus anti-CD28 antibody (4 μg/ml). 100 μl cell suspension was added per well. 100 μl of R10F medium was then added per well to give a final volume of 200 μl. (2×10⁵ cells/well, anti-CD28 final concentration 2 μg/ml). The plates were then incubated at 37° C. for 72 hours. After 72 hours supernatants were removed for ELISA as described in Example 3 (primary stimulation).

Re-Stimulation with APC Plus Anti-CD3

Primary stimulated cells from Example 3 were harvested after 7 days and restimulated with APCs of the same strain (2×10⁴ per well) plus anti-CD3 antibody.

Mouse spleen cells were isolated as described in Example 1 up to the counting step. Thy-1.2 antibody-binding cells were then removed on a MACS column and the flowthrough was recovered and treated with mitomycin-C for 45 minutes then added to a 96 well plate in 100 μl R10F medium with equal numbers of cells from Example 3 and 0.5 μg/ml anti-CD3 antibody.

Cell proliferation was measured using a kit from Roche Molecular Biochemicals, Cell Proliferation ELISA, BrdU (chemiluminescent) 1 669 915, according to the manufacturer's instructions. Plates were pulsed at 72 hours and read on a luminometer.

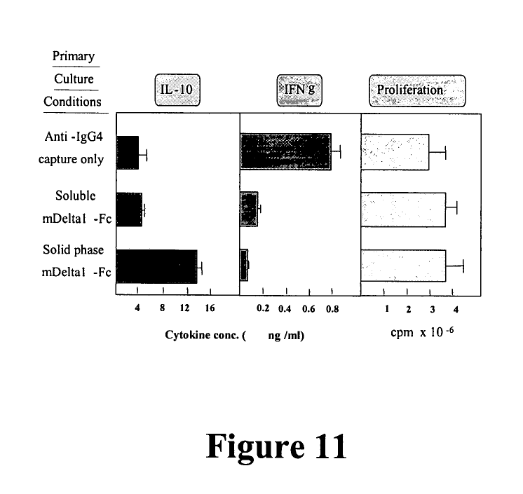

Cytokines (IL-10 and IFN-γ) were measured as described in Example 3. Results are shown in FIG. 11.

### Example 8

### CHO—N2 (N27) Luciferase Reporter Assay

The 2 ml of transfection mixture was added to the flask of cells containing 8 ml of medium and the resulting mixture was left in a CO₂ incubator overnight before removing the transfected cells and adding to the 96-well plate containing the immobilised Notch ligand protein.

N27#11 cells (CHO cells expressing full length human Notch2 and a CBF1-luciferase reporter construct; T₈₀ flask; as described in WO 03/012441, Lorantis, e.g. see Example 7 therein) were removed using 0.02% EDTA solution (Sigma), spun down and resuspended in 10 ml DMEM plus 10% (HI)FCS plus glutamine plus P/S. 10 μl of cells were counted and the cell density was adjusted to 2.0×10⁵ cells/ml with fresh DMEM plus 10% (HI)FCS plus glutamine plus P/S. 100 μl per well was added to a 96-well tissue culture plate (flat bottom), i.e. 2.0×10⁴ transfected cells per well, using a multi-channel pipette and the plate was then incubated overnight.

A) Immobilisation of Notch Ligand Protein Directly onto a 96-Well Tissue Culture Plate

10 μg of purified Notch ligand protein was added to sterile PBS in a sterile Eppendorf tube to give a final volume of 1 ml. Serial 1:2 dilutions were made by adding 500 μl into sterile Eppendorf tubes containing 500 μl of sterile PBS to generate dilutions of 10 μg/ml, 5 μg/ml, 2.5 μg/ml, 1.25 μg/ml, 0.625 μg/ml and 0 μg/ml.

The lid of the plate was sealed with parafilm and the plate was left at 4° C. overnight or at 37° C. for 2 hours. The protein was then removed and the plate was washed with 200 μl of PBS.

B) A20-Delta Cells

The IVS, IRES, Neo and pA elements were removed from plasmid pIRESneo2 (Clontech, USA) and inserted into a pUC cloning vector downstream of a chicken beta-actin promoter (e.g. see GenBank Accession No E02199). Mouse Delta-1 (e.g. see GenBank Accession No NM_{—}007865) was inserted between the actin promoter and IVS elements and a sequence with multiple stop codons in all three reading frames was inserted between the Delta and IVS elements.

The resulting construct was transfected into A20 cells using electroporation and G418 to provide A20 cells expressing mouse Delta1 on their surfaces (A20-Delta).

C)CHO and CHO-hDelta1-V5-His Assay Control

CHO cells were maintained in DMEM plus 10% (HI)FCS plus glutamine plus P/S and CHO-hDelta1-V5-His (clone#10) cells were maintained in DMEM plus 10% (HI)FCS plus glutamine plus P/S plus 0.5 mg/ml G418.

Cells were removed using 0.02% EDTA solution (Sigma), spun down and resuspended in 10 ml DMEM plus 10% (HI)FCS plus glutamine plus P/S. 10 μl of cells were counted and the cell density was adjusted to 5.0×10⁵ cells/ml with fresh DMEM plus 10% (HI)FCS plus glutamine plus P/S. 300 μl of each cell line at 5.0×10⁵ cells/ml was added into duplicate wells of a 96-well tissue culture plate. 150 μl of DMEM plus 10% (HI)FCS plus glutamine plus P/S was added in to the next 5 wells below each well. 150 μl of cells were serially diluted into the next 4 wells giving cell density dilution of 5.0×10⁵ cells/ml, 2.5×10⁵ cells/ml, 1.25×10⁵ cells/ml, 0.625×10⁵ cells/ml, 0.3125×10⁵ cells/ml and 0 cells/ml.

100 μl from each well was added into the 96-well plate containing 100 μl of CHO—N2 cells transfected with 10xCBF1-Luc (2.0×10⁴ transfected CHO—N2 cells/well) and the plate was left in an incubator overnight.

D) Cell Co-Culture

5×10⁴ CHO—N2 cells were plated on a 96 well plate. CHO-Delta or A20-Delta cells were titrated in as required (max ratio CHO—N2: CHO-Delta was 1:1, max ratio CHO—N2: A20-Delta was 1:2). The mixture was incubated overnight before conducting a luciferase assay.

E) Luciferase Assay

Supernatant was removed from all wells. 100 μl of PBS and 100 μl of SteadyGlo™ luciferase assay reagent (Promega) was added and the cells were left at room temperature for 5 minutes. The mixture was pipetted up and down 2 times to ensure cell lysis and contents from each well were transferred into a white 96-well OptiPlate™ (Packard). Luminescence was measured in a TopCount™ counter (Packard).

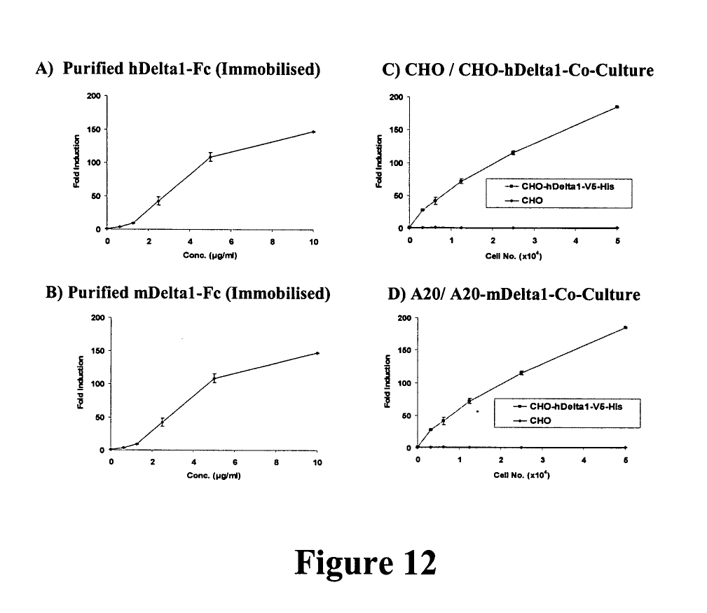

Results of sample assays are shown in FIGS. 12 A to D.

### Example 9

### Dynabeads Luciferase Assay Method for Detecting Notch Ligand Activity

Fc-tagged Notch ligands were immobilised on Streptavidin-Dynabeads (CELLection Biotin Binder Dynabeads [Cat. No. 115.21] at 4.0×10⁸ beads/ml from Dynal (UK) Ltd; beads) in combination with biotinylated α-IgG-4 (clone JDC14 at 0.5 mg/ml from Pharmingen [Cat. No. 555879]) as follows:

2.5×10⁷ beads (62.5 μl of beads at 4.0×10⁸ beads/ml) and 5 μg biotinylated α-IgG-4 was used for each sample assayed. PBS was added to the beads to 1 ml and the mixture was spun down at 13,000 rpm for 1 minute. Following washing with a further 1 ml of PBS the mixture was spun down again. The beads were then resuspended in a final volume of 100 μl of PBS containing the biotinylated α-IgG-4 in a sterile Eppendorf tube and placed on shaker at room temperature for 30 minutes. PBS to was added to 1 ml and the mixture was spun down at 13,000 rpm for 1 minute and then washed twice more with 1 ml of PBS.

The mixture was then spun down at 13,000 rpm for 1 minute and the beads were resupsended in a 50 μl PBS per sample. 50 μl of biotinylated α-IgG-4-coated beads were added to each sample and the mixture was incubated on a rotary shaker at 4° C. overnight. The tube was then spun at 1000 rpm for 5 minutes at room temperature.

The beads then were washed with 10 ml of PBS, spun down, resupended in 1 ml of PBS, transferred to a sterile Eppendorf tube, washed with a further 2×1 ml of PBS, spun down and resuspended beads in a final volume of 250 μl of DMEM plus 10% (HI)FCS plus glutamine plus P/S, i.e. at 1.0×10⁵ beads/μl.

Stable N27#11 cells from Example 8 (T₈₀ flask) were removed using 0.02% EDTA solution (Sigma), spun down and resuspended in 10 ml DMEM plus 10% (HI)FCS plus glutamine plus P/S. 10 μl of cells were counted and the cell density was adjusted to 1.0×10⁵ cells/ml with fresh DMEM plus 10% (HI)FCS plus glutamine plus P/S. 1.0×10⁵ of the cells were plated out per well of a 24-well plate in a 1 ml volume of DMEM plus 10% (HI)FCS plus glutamine plus P/S and cells were placed in an incubator to settle down for at least 30 minutes.

100 μl of beads were then added in duplicate to the first pair of wells to give 1.0×10⁷ beads/well (100 beads/cell); 20 μl of beads added in duplicate to the second pair of wells to give 2.0×10⁶ beads/well (20 beads/cell); 4 μl of beads added in duplicate to the third pair of wells to give 4.0×10⁵ beads/well (4 beads/cell) and 0 μl of beads added to the fourth pair of wells. The plate was left in a CO₂ incubator overnight.

Luciferase Assay

Supernatant was then removed from all the wells, 150 μl of PBS and 150 μl of SteadyGlo luciferase assay reagent (Promega) were added and the resulting mixture left at room temperature for 5 minutes.

The mixture was then pipetted up and down 2 times to ensure cell lysis and the contents from each well were transferred into an Eppendorf tube, spun at 13,000 rpm for 1 minute and the cleared supernatant was transferred to a white 96-well OptiPlate™ (Packard), leaving the bead pellet behind. Luminescence was then read in a TopCount™ (Packard) counter.

### Example 10

### Dynabeads ELISA Assay Method for Detecting Notch Ligand Activity

M450 Streptavidin Dynabeads were coated with anti-hamster-IgG1 biotinylated monoclonal antibody, anti-human-IgG4 biotinylated monoclonal antibody or both antibodies and rotated for 2 hours at room temperature.

Beads were washed three times with PBS (1 ml). The anti-hamster-IgG1 beads were then further incubated with anti-CD3ε chain monoclonal antibody, the anti-human-IgG4 beads were further incubated with Fc-Delta, and the double coated beads incubated with both anti-CD3ε chain monoclonal antibody and Fc-Delta. Beads were rotated overnight at 4° C., washed three times with PBS (1 ml) and resuspended.

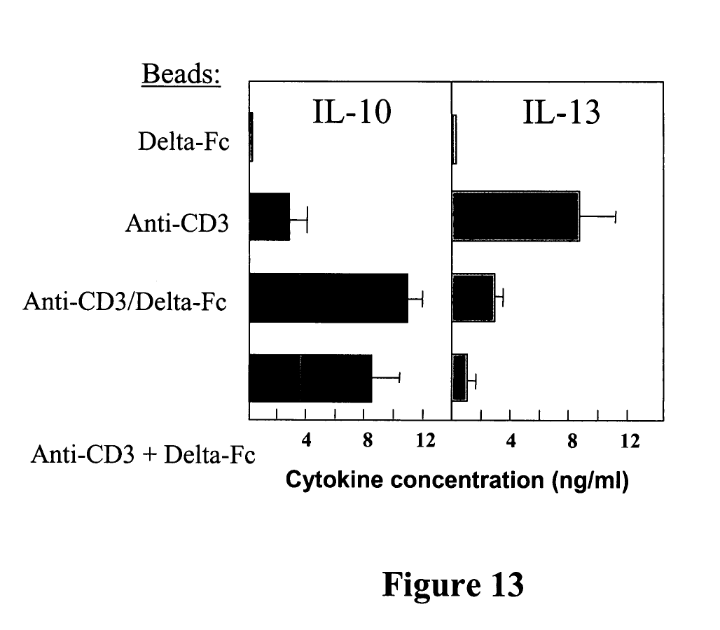

T-cell assays were carried out with CD4+ T-cells and the beads. Supernatants were removed after 72 hours and cytokines measured by ELISA as described in Example 3. Results are shown in FIG. 13.

### Example 11

### Modulation of Cytokine Production by Human CD4+ T Cells in the Presence of Delta1-hIgG4 Immobilised on Dynal Microbeads

Human peripheral blood mononuclear cells (PBMC) were purified from blood using Ficoll-Paque separation medium (Pharmacia). Briefly, 28 ml of blood were overlaid on 21 ml of Ficoll-Paque separation medium and centrifuged at 18-20° C. for 40 minutes at 400 g. PBMC were recovered from the interface and washed 3 times before use for CD4+ T cell purification.

Human CD4+ T cells were isolated by positive selection using anti-CD4 microbeads from Miltenyi Biotech according to the manufacturer's instructions.

The CD4+ T cells were incubated in triplicates in a 96-well-plate (flat bottom) at 10⁵ CD4/well/200 μl in RPMI medium containing 10% FCS, glutamine, penicillin, streptomycin and β₂-mercaptoethanol.

Cytokine production was induced by stimulating the cells with anti-CD3/CD28 T cell expander beads from Dynal at a 1:1 ratio (bead/cell) or plate bound anti-CD3 (clone UCHT1, BD Biosciences, 5 μg/ml) and soluble anti-CD28 (clone CD28.2, BD Biosciences, 2 μg/ml). Beads coated with mouse or human Delta1EC domain-hIgG4 fusion protein (prepared as described above with the modifications that incubation with human IgG4 was for 30-40 minutes at room temperature and incubation with Delta-Fc was for two hours at room temperature) or control beads were added in some of the wells at a 10:1 ratio (beads/cell). The supernatants were removed after 3 or 4 days of incubation at 37° C./5% CO₂/humidified atmosphere and cytokine production was evaluated by ELISA using Pharmingen kits OptEIA Set human IL10 (catalog No. 555157), OptEIA Set human IL-5 (catalog No. 555202) and OptEIA Set human IFNg (catalog No 555142) for IL-10, IL-5 and IFNg respectively and a human TNFa DuoSet from R&D Systems (catalog. No. DY210) for TNFa according to the manufacturer's instructions.

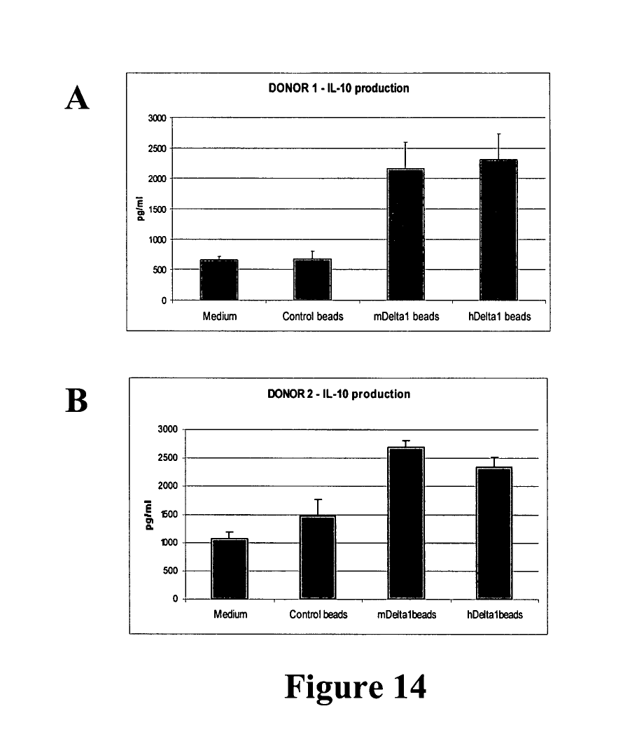

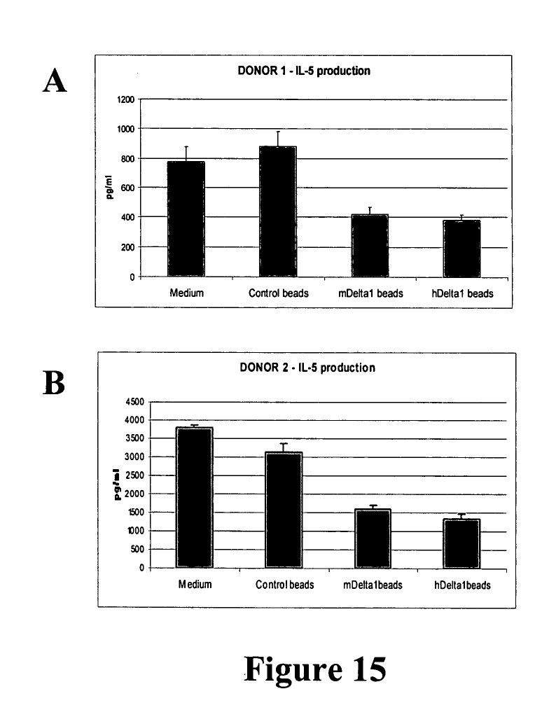

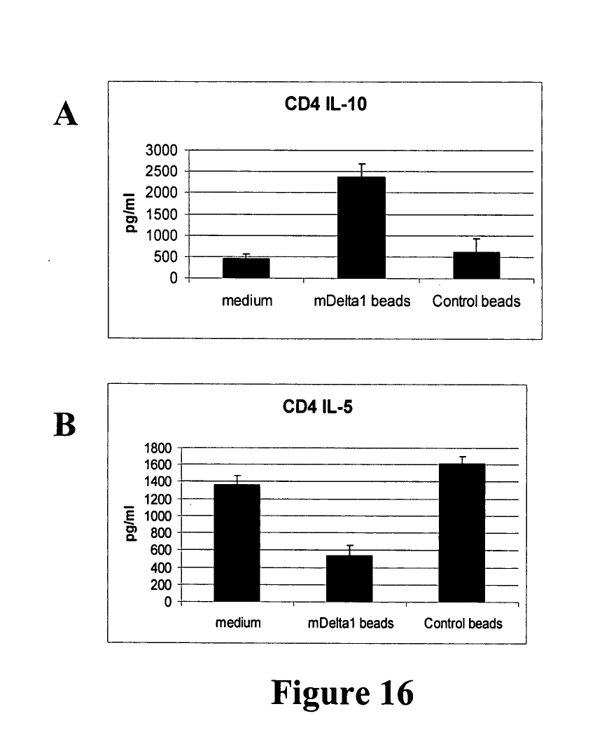

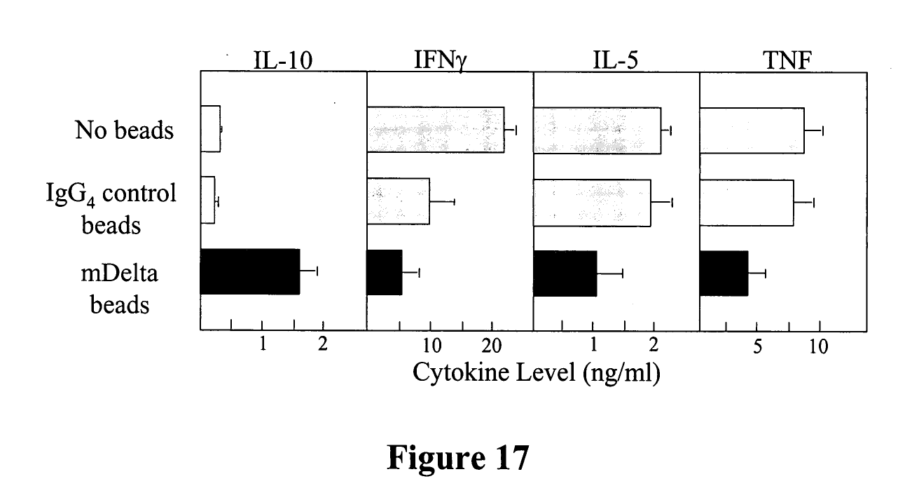

Results are shown in FIGS. 14 to 17.

### Example 12

### Variation of Bead:Cell Ratios

The procedure of Example 11 was repeated except that the ratio of control beads to cells and mouse Delta1-hIgG4 fusion protein coated beads to cells was varied between 16:1 and 0.25:1 (variously 16:1, 8:1, 4:1, 2:1, 1:1, 0.5:1, 0.25:1) and human Delta1-hIgG4 fusion protein coated beads were also used at the same ratios for comparison.

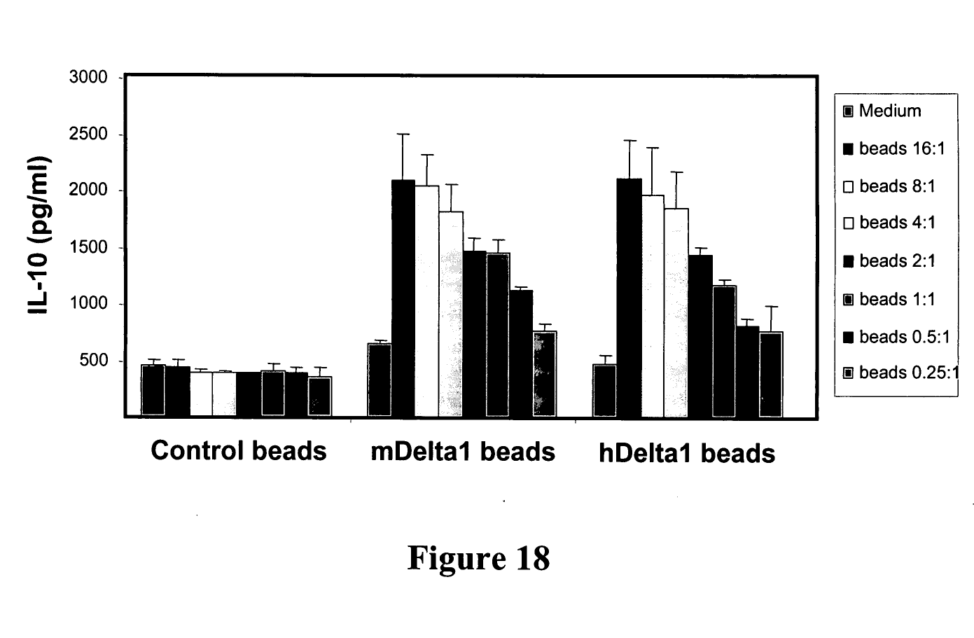

Results are shown in FIG. 18.

### Example 13

### Comparison of CD45RO+ (Memory Cells) and CD45RO− (Naive Cells)

The procedure of Example 11 was repeated except that prior to the stimulation the human CD4+ were separated into CD45RO+ (memory cells) and CD45RO− (naive cells, data not shown on the slide). The magnetic separation was done using anti-CD4 Multisort microbeads (cat. No. 551-01) and then anti-CD45RO microbeads (cat. No. 460-01) supplied by Miltenyi Biotech and following Miltenyi's protocol.

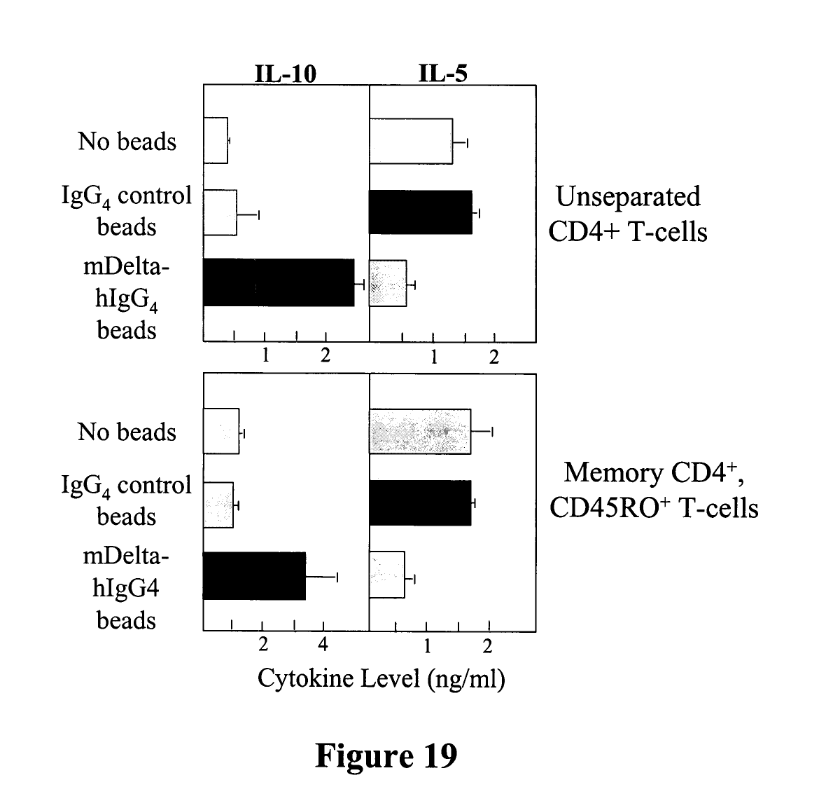

Results are shown in FIG. 19.

### Example 14

### Measurement of Cytokine Production in Stimulated Mouse CD4+ Cells Under Polarising Conditions

(i) CD4+ Cell Purification

Spleens were removed from mice (variously Balb/c females, 8-10 weeks, C57B/6 females, 8-10 weeks, CARD1 females, 8-10 weeks (D011.10 transgenic, CAR transgenic)) and passed through a 0.2 μM cell strainer into 20 ml R10F medium (R10F-RPMI 1640 media (Gibco Cat No 22409) plus 2 mM L-glutamine, 50 μg/ml Penicillin, 50 μg/ml Streptomycin, 5×10⁻⁵ M β-mercapto-ethanol in 10% fetal calf serum). The cell suspension was spun (1150 rpm 5 min) and the media removed.

The cells were incubated for 4 minutes with 5 ml ACK lysis buffer (0.15M NH₄Cl, 1.0M KHCO₃, 0.1 mM Na₂EDTA in double distilled water) per spleen (to lyse red blood cells). The cells were then washed once with R10F medium and counted. CD4+ cells were purified from the suspensions by positive selection on a Magnetic Associated Cell Sorter (MACS) column (Miltenyi Biotec, Bisley, UK: Cat No 130-042-401) using CD4 (L3T4) beads (Miltenyi Biotec Cat No 130-049-201), according to the manufacturer's directions.

(ii) Antibody Coating

96 well flat-bottomed plates were coated with Dulbecco's Phosphate Buffered Saline (DPBS) plus 1 μg/ml anti-CD3 antibody (Pharmingen, San Diego, US: Cat No 553058, Clone No 145-2C11) plus 1 μg/ml anti-IgG4 antibody (Pharmingen Cat No 555878). 100 μl of coating mixture was used per well. Plates were incubated overnight at 4° C. then washed with DPBS. Each well then received either 100 μl 1 DPBS or 100 μl DPBS plus 10 μg/ml Notch ligand (mouse Delta 1 extracellular domain/Ig4Fc fusion protein; Fc-delta). The plates were incubated for 2-3 hours at 37° C. then washed again with DPBS before cells (prepared as in (i)) were added.

(iii) Primary Polyclonal Stimulation

CD4+ cells were cultured in 96 well, flat-bottomed plates pre-coated as in (ii) above. Cells were re-suspended, following counting, at 2×10⁶/ml in R10F medium plus 4 μg/ml anti-CD28 antibody (Pharmingen, Cat No 553294, Clone No 37.51). 100 μl cell suspension was added per well. 100 μl of polarising or control medium was then added to each well to give a final volume of 200 μl (2×10⁵ cells/well, anti-CD28 final concentration 2 μg/ml) as follows:

Un-polarised cells: R10F medium.

Th1 polarised cells: R10F medium plus anti-IL-4 antibody (10 □g/ml, Pharmingen Cat No 554432) plus IL-12 (10 ng/ml, Peprotech 210-12).

Th2 polarised cells: R10F medium plus anti-IL-12 antibody (10 μg/ml, Pharmingen Cat No 554475) plus anti-IFNg antibody (1 μg/ml, Pharmingen Cat No 554408) plus IL-4 (10 ng/ml, Peprotech Cat No 214-14).

The plates were then incubated at 37° C. for 72 hours.

125 μl supernatant was then removed from each well and stored at −20° C. until tested by ELISA for IL-10 and TNFa using antibody pairs from R & D Systems (Abingdon, UK). The cells were then split 1 in 3 into new wells (not coated) and fed with R10F medium plus recombinant human IL-2 (2.5 ng/ml, PeproTech Inc, London, UK: Cat No 200-02).

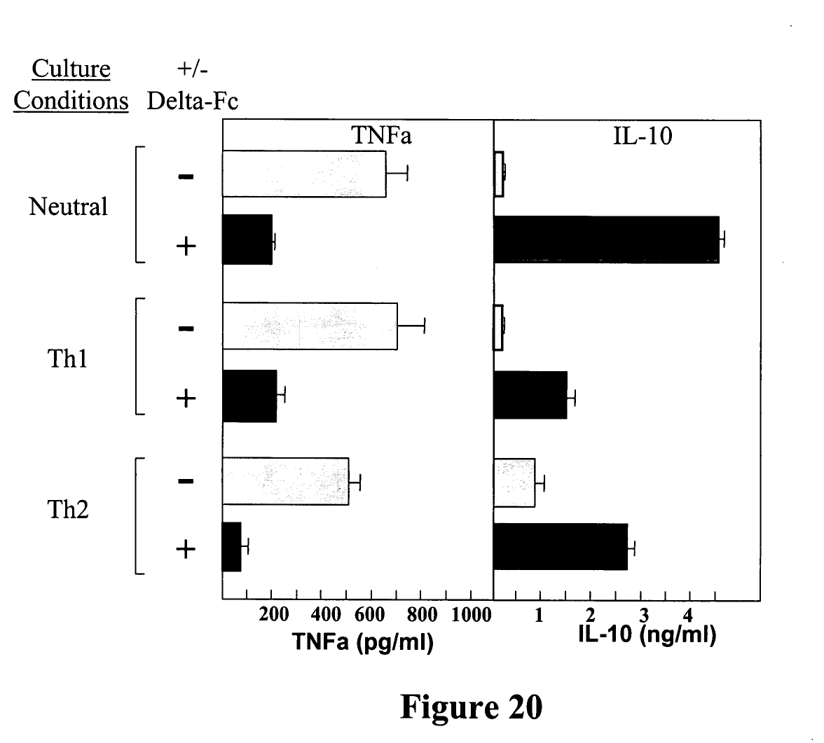

Results are shown in FIG. 20.

### Example 15

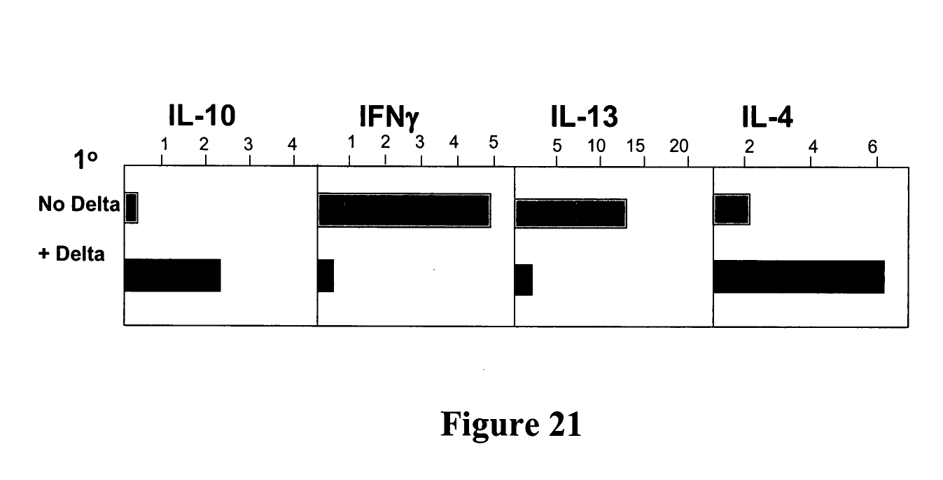

The effect of Notch activation by Delta protein on IL-4 expression was determined generally as described above. Results are shown in FIG. 21.

### Example 16

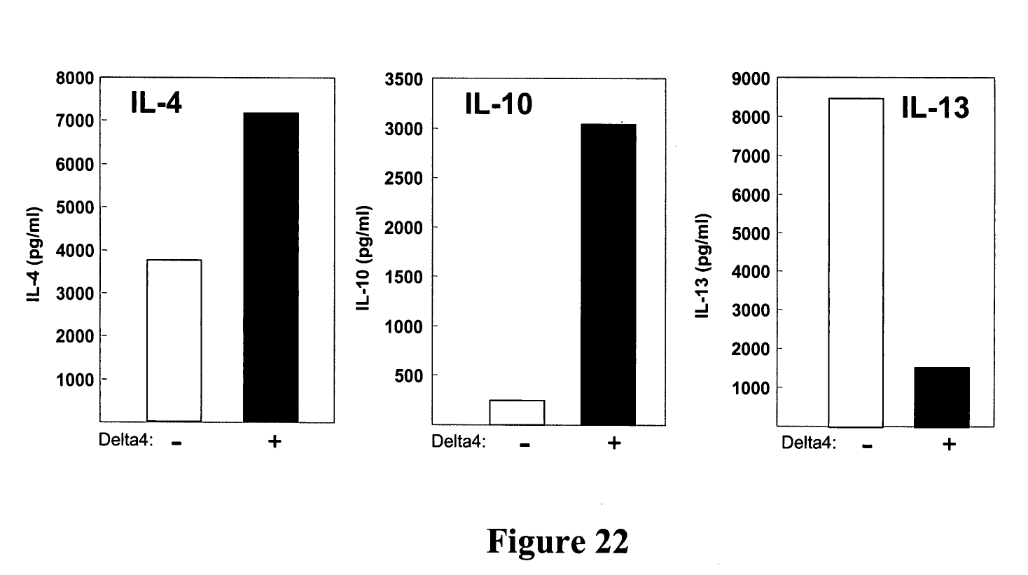

Delta4 protein (R& D Systems, 10 mg/ml) was bound to plates generally as described above and effects on IL-4, IL-10 and IL-13 secretion by anti-CD3/28 activated mouse T-cells were measured. Results are shown in FIG. 22.

### Example 17

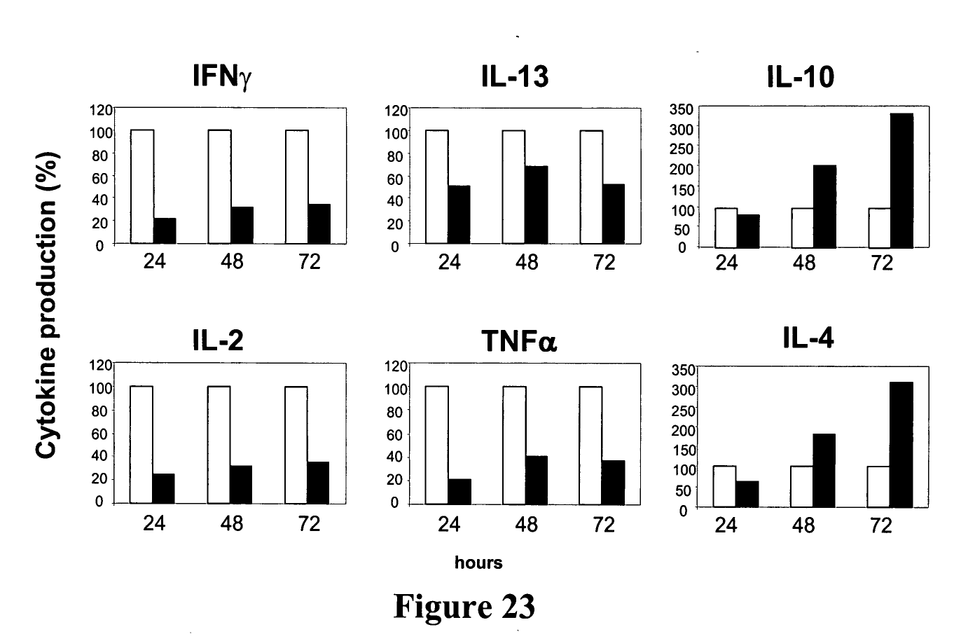

Cytokine up- or down-regulation by Delta1 protein (in anti-CD3/28 activated mouse T-cells) was measured over time. Cytokine levels sampled at each time point were normalized to levels in the absence of Delta. Results are shown in FIG. 23.

### Example 18

Transcription factor expression by anti-CD3/28 activated mouse T-cells activated under neutral, Th1 or Th2 culture conditions was measured with or without Delta1 protein.

RNA was isolated from cell samples using the Qiagen RNeasy system. First strand cDNA was synthesised from 500 ng of total RNA with SuperScriptII (InVitrogen) using a mixture of oligo-d(T) and random decamers (Invitrogen, Paisley, UK). Q-PCR was performed using SYBR green detection chemistry on the Roche Lightcycler system. Gene-specific quantification was achieved by comparing each cDNA to a serially-diluted standard plasmid containing the relevant amplicon. Relative expression levels for the gene of interest were expressed by dividing by the calculated expression level for the 18S rRNA housekeeping gene. Primer sequences used were as follows (5′ to 3′, forward followed by reverse):

| | | |
|---|---|---|
| 18S rRNA: | GTAACCCGTTGAACCCCATT | (SEQ ID NO:32) |
| and | | |
| | CCATCCAATCGGTAGTAGCG; | (SEQ ID NO:33) |
| IL-10: | CCAGGGAGATCCTTTGATGA | (SEQ ID NO:34) |
| and | | |
| | CATTCCCAGAGGAATTGCAT; | (SEQ ID NO:35) |
| IFN-γ: | GTGATTGCGGGGTTGTATCT | (SEQ ID NO:36) |
| and | | |
| | ACATCTCCTCCCATCAGCAG; | (SEQ ID NO:37) |
| Tbet: | GATCGTCCTGCAGTCTCTCC | (SEQ ID NO:38) |
| and | | |
| | AGTTCTCCCGGAATCCTTTG; | (SEQ ID NO:39) |
| c-Maf: | AGAGGGTGCAGCAGAGACAC | (SEQ ID NO:40) |
| and | | |
| | CATGAAAAATTCGGGAGAGG; | (SEQ ID NO:41) |
| GATA-3: | CTGGAGGAGGAACGCTAATG | (SEQ ID NO:42) |
| and | | |
| | GTTGAAGGAGCTCTTGG. | (SEQ ID NO:43) |

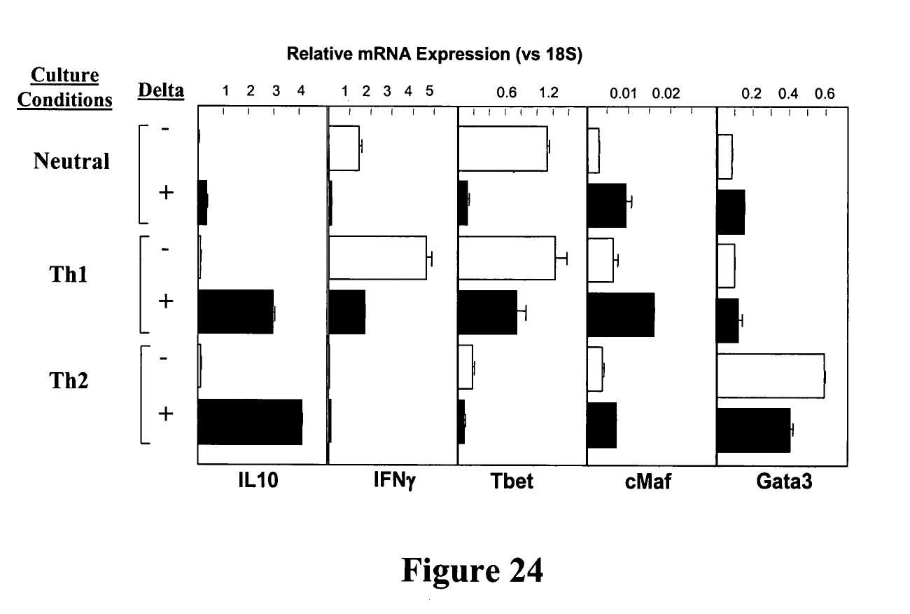

Results are shown in FIG. 24.

The invention is further described by the following numbered paragraphs:

1. A method for modifying IL-4 expression by administering a modulator of Notch signalling.

2. A method for increasing IL-4 expression by administering an activator of Notch signalling.

3. A method for reducing IL-4 expression by administering an inhibitor of Notch signalling.

4. A method for increasing IL-4 expression by administering a Notch receptor agonist.

5. A method for reducing IL-4 expression by administering a Notch receptor antagonist.

6. A method as described in any one of the preceding paragraphs wherein the modulator of Notch signalling modifies cytokine expression in leukocytes, fibroblasts or epithelial cells.

7. A method as described in paragraph 1 wherein the modulator of Notch signalling modifies cytokine expression in lymphocytes or macrophages.

8. A method for modifying the TH1/TH2 balance of an immune response away from a TH1 response by administering a modulator of Notch signalling.

9. A method for modifying the TH1/TH2 balance of an immune response away from a TH1 response by administering an activator of a Notch receptor.

10. A method for modifying the TH1/TH2 balance of an immune response in favour of a TH2 response by administering a modulator of Notch signalling.

11. A method for modifying the TH1/TH2 balance of an immune response in favour of a TH2 response by administering an activator of a Notch receptor.

12. A method as described in any one of the preceding paragraphs to treat graft rejection

13. A method as described in any one of the preceding paragraphs to treat autoimmune disease

14. A method as described in paragraph 13, wherein the autoimmune disorder is selected from the group consisting of multiple sclerosis, insulin-dependent diabetes, sympathetic ophthalmia, uveitis and psoriasis.

15. A method for generating an immune modulatory cytokine profile with increased IL-10 expression and increased IL-4 expression by administering a modulator of Notch signalling.

16. A method for generating an immune modulatory cytokine profile with increased IL-4 expression and reduced IL-5, IL-13 and TNFα expression by administering a modulator of Notch signalling.

17. A method for generating an immune modulatory cytokine profile with increased IL-4 expression and reduced IL-2, IFNγ, IL-5, IL-13 and TNFα expression by administering a modulator of Notch signalling.

18. A method as described in paragraph 16 or paragraph 17 wherein the cytokine profile also exhibits increased IL-10 expression.

19. A method for increasing a TH2 immune response by administering a modulator of Notch signalling to increase IL-4 expression.

20. A method for reducing a TH1 immune response by administering a modulator of Notch signalling to increase IL-4 expression.

21. A method for increasing a TH2 immune response and reducing a TH1 immune response by administering a modulator of Notch signalling to increase IL-4 expression.

22. A method for treating inflammation or an inflammatory condition by administering a modulator of Notch signalling to increase IL-4 expression and reduce a TH1 immune response.

23. A method for treating inflammation or an inflammatory or autoimmune condition by administering a modulator of Notch signalling to increase IL-4 expression and reduce a TH1 immune response.

24. A method as described in any one of the preceding paragraphs wherein the modulator of Notch signalling is administered to a patient in vivo.

25. A method as described in any one of paragraphs 1 to 24 wherein the modulator of Notch signalling is administered to a cell ex-vivo, after which the cell may be administered to a patient.

26. A method as described in any one of the preceding paragraphs to treat a disorder selected from the group consisting of: thyroiditis, insulitis, multiple sclerosis, iridocyclitis, uveitis, orchitis, hepatitis, Addison's disease, myasthenia gravis, rheumatoid arthritis, lupus erythematosus, immune hyperreactivity, insulin dependent diabetes mellitus, anemia (aplastic, hemolytic), autoimmune hepatitis, skleritis, idiopathic thrombocytopenic purpura, inflammatory bowel diseases (Crohn's disease, ulcerative colitis), juvenile arthritis, scleroderma and systemic sclerosis, sjogren's syndrom, undifferentiated connective tissue syndrome, antiphospholipid syndrome, vasculitis (polyarteritis nodosa, allergic granulomatosis and angiitis, Wegner's granulomatosis, Kawasaki disease, hypersensitivity vasculitis, Henoch-Schoenlein purpura, Behcet's Syndrome, Takayasu arteritis, Giant cell arteritis, Thrombangiitis obliterans), polymyalgia rheumatica, essentiell (mixed) cryoglobulinemia, Psoriasis vulgaris and psoriatic arthritis, diffus fasciitis with or without eosinophilia, polymyositis and other idiopathic inflammatory myopathies, relapsing panniculitis, relapsing polychondritis, lymphomatoid granulomatosis, erythema nodosum, ankylosing spondylitis, Reiter's syndrome, inflammatory dermatitis, unwanted immune reactions and inflammation associated with arthritis, including rheumatoid arthritis, inflammation associated with hypersensitivity and allergic reactions, systemic lupus erythematosus, collagen diseases, inflammation associated with atherosclerosis, arteriosclerosis, atherosclerotic heart disease, reperfusion injury, cardiac arrest, myocardial infarction, vascular inflammatory disorders, respiratory distress syndrome or other cardiopulmonary diseases, inflammation associated with peptic ulcer, ulcerative colitis and other diseases of the gastrointestinal tract, hepatic fibrosis, liver cirrhosis or other hepatic diseases, thyroiditis or other glandular diseases, glomerulonephritis or other renal and urologic diseases, otitis or other oto-rhino-laryngological diseases, dermatitis or other dermal diseases, periodontal diseases or other dental diseases, orchitis or epididimo-orchitis, infertility, orchidal trauma or other immune-related testicular diseases, placental dysfunction, placental insufficiency, habitual abortion, eclampsia, pre-eclampsia and other immune and/or inflammatory-related gynaecological diseases, posterior uveitis, intermediate uveitis, anterior uveitis, conjunctivitis, chorioretinitis, uveoretinitis, optic neuritis, intraocular inflammation, e.g. retinitis or cystoid macular oedema, sympathetic ophthalmia, scleritis, retinitis pigmentosa, immune and inflammatory components of degenerative fondus disease, inflammatory components of ocular trauma, ocular inflammation caused by infection, proliferative vitreo-retinopathies, acute ischaemic optic neuropathy, excessive scarring, e.g. following glaucoma filtration operation, immune and/or inflammation reaction against ocular implants and other immune and inflammatory-related ophthalmic diseases, inflammation associated with autoimmune diseases or conditions or disorders where, both in the central nervous system (CNS) or in any other organ, immune and/or inflammation suppression would be beneficial, Parkinson's disease, complication and/or side effects from treatment of Parkinson's disease, AIDS-related dementia complex HIV-related encephalopathy, Devic's disease, Sydenham chorea, Alzheimer's disease and other degenerative diseases, conditions or disorders of the CNS, inflammatory components of strokes, post-polio syndrome, immune and inflammatory components of psychiatric disorders, myelitis, encephalitis, subacute sclerosing pan-encephalitis, encephalomyelitis, acute neuropathy, subacute neuropathy, chronic neuropathy, Guillaim-Barre syndrome, Sydenham chora, pseudo-tumour cerebri, Down's Syndrome, Huntington's disease, amyotrophic lateral sclerosis, inflammatory components of CNS compression or CNS trauma or infections of the CNS, inflammatory components of muscular atrophies and dystrophies, and immune and inflammatory related diseases, conditions or disorders of the central and peripheral nervous systems, post-traumatic inflammation, septic shock, infectious diseases, inflammatory complications or side effects of surgery or organ, inflammatory and/or immune complications and side effects of gene therapy, e.g. due to infection with a viral carrier, or inflammation associated with AIDS, to suppress or inhibit a humoral and/or cellular immune response, to treat or ameliorate monocyte or leukocyte proliferative diseases, e.g. leukaemia, by reducing the amount of monocytes or lymphocytes, for the prevention and/or treatment of graft rejection in cases of transplantation of natural or artificial cells, tissue and organs such as cornea, bone marrow, organs, lenses, pacemakers, natural or artificial skin tissue.

27. Use of a modulator of Notch signalling to modify IL-4 expression.

28. Use of an activator of Notch signalling to increase IL-4 expression.

29. Use of an inhibitor of Notch signalling to reduce IL-4 expression.

30. Use of a Notch receptor agonist to increase IL-4 expression.

31. Use of a Notch receptor antagonist to reduce IL-4 expression.

32. A use as described in any one of paragraphs 27 to 31 wherein the modulator of Notch signalling modifies cytokine expression in leukocytes, fibroblasts or epithelial cells.

33. A use as described in paragraph 32 wherein the modulator of Notch signalling modifies cytokine expression in dendritic cells, lymphocytes or macrophages, or their progenitors or tissue-specific derivatives.

34. Use of a modulator of Notch signalling for modifying the TH1/TH2 balance of an immune response away from a TH1 response.

35. Use of a modulator of Notch signalling for modifying the TH1/TH2 balance of an immune response away from a TH1 response.

36. Use of a modulator of Notch signalling for modifying the TH1/TH2 balance of an immune response in favour of a TH2 response.

37. Use of an activator of Notch signalling for modifying the TH1/TH2 balance of an immune response in favour of a TH2 response.

38. A use as described in paragraph 37 wherein the activator of Notch signalling is a Notch receptor agonist or partial agonist.

39. A use as described in any one of paragraphs 27 to 38 to treat graft rejection

40. A use as described in any one of paragraphs 27 to 38 to treat autoimmune disease

41. A use as described in paragraph 40, wherein the autoimmune disorder is selected from the group consisting of multiple sclerosis, insulin-dependent diabetes, sympathetic ophthalmia, uveitis and psoriasis.

42. The use of a modulator of Notch signalling to generate an immune modulatory cytokine profile with increased IL-10 expression and increased IL-4 expression.

43. The use of a modulator of Notch signalling to generate an immune modulatory cytokine profile with increased IL-4 expression and reduced IL-5, IL-13 and TNFα expression.

44. The use of a modulator of Notch signalling to generate an immune modulatory cytokine profile with increased IL-4 expression and reduced IL-2, IFNγ, IL-5, IL-13 and TNFα expression.

45. A use as described in paragraph 43 or paragraph 44 wherein the cytokine profile also exhibits increased IL-10 expression.

46. The use of a modulator of Notch signalling for increasing a TH2 immune response.

47. The use of a modulator of Notch signalling for reducing a TH1 immune response.

48. The use of a modulator of Notch signalling for increasing a TH2 immune response and reducing a TH1 immune response.

49. The use of a modulator of Notch signalling for treating inflammation or an inflammatory condition by increasing IL-4 expression and reducing a TH1 immune response.

50. The use of a modulator of Notch signalling for treating inflammation or an inflammatory or autoimmune condition by increasing IL-4 expression and reducing a TH1 immune response.

51. A use as described in any one of paragraphs 27 to 50 wherein the modulator of Notch signalling is administered to a patient in vivo.

52. A use as described in any one of paragraphs 27 to 50 wherein the modulator of Notch signalling is administered to a cell ex-vivo, after which the cell may be administered to a patient.

53. A use as described in any one of the paragraphs 27 to 50 to treat a disorder selected from the group consisting of: thyroiditis, insulitis, multiple sclerosis, iridocyclitis, uveitis, orchitis, hepatitis, Addison's disease, myasthenia gravis, rheumatoid arthritis, lupus erythematosus, immune hyperreactivity, insulin dependent diabetes mellitus, anemia (aplastic, hemolytic), autoimmune hepatitis, skleritis, idiopathic thrombocytopenic purpura, inflammatory bowel diseases (Crohn's disease, ulcerative colitis), juvenile arthritis, scleroderma and systemic sclerosis, sjogren's syndrom, undifferentiated connective tissue syndrome, antiphospholipid syndrome, vasculitis (polyarteritis nodosa, allergic granulomatosis and angiitis, Wegner's granulomatosis, Kawasaki disease, hypersensitivity vasculitis, Henoch-Schoenlein purpura, Behcet's Syndrome, Takayasu arteritis, Giant cell arteritis, Thrombangiitis obliterans), polymyalgia rheumatica, essentiell (mixed) cryoglobulinemia, Psoriasis vulgaris and psoriatic arthritis, diffus fasciitis with or without eosinophilia, polymyositis and other idiopathic inflammatory myopathies, relapsing panniculitis, relapsing polychondritis, lymphomatoid granulomatosis, erythema nodosum, ankylosing spondylitis, Reiter's syndrome, inflammatory dermatitis, unwanted immune reactions and inflammation associated with arthritis, including rheumatoid arthritis, inflammation associated with hypersensitivity and allergic reactions, systemic lupus erythematosus, collagen diseases, inflammation associated with atherosclerosis, arteriosclerosis, atherosclerotic heart disease, reperfusion injury, cardiac arrest, myocardial infarction, vascular inflammatory disorders, respiratory distress syndrome or other cardiopulmonary diseases, inflammation associated with peptic ulcer, ulcerative colitis and other diseases of the gastrointestinal tract, hepatic fibrosis, liver cirrhosis or other hepatic diseases, thyroiditis or other glandular diseases, glomerulonephritis or other renal and urologic diseases, otitis or other oto-rhino-laryngological diseases, dermatitis or other dermal diseases, periodontal diseases or other dental diseases, orchitis or epididimo-orchitis, infertility, orchidal trauma or other immune-related testicular diseases, placental dysfunction, placental insufficiency, habitual abortion, eclampsia, pre-eclampsia and other immune and/or inflammatory-related gynaecological diseases, posterior uveitis, intermediate uveitis, anterior uveitis, conjunctivitis, chorioretinitis, uveoretinitis, optic neuritis, intraocular inflammation, e.g. retinitis or cystoid macular oedema, sympathetic ophthalmia, scleritis, retinitis pigmentosa, immune and inflammatory components of degenerative fondus disease, inflammatory components of ocular trauma, ocular inflammation caused by infection, proliferative vitreo-retinopathies, acute ischaemic optic neuropathy, excessive scarring, e.g. following glaucoma filtration operation, immune and/or inflammation reaction against ocular implants and other immune and inflammatory-related ophthalmic diseases, inflammation associated with autoimmune diseases or conditions or disorders where, both in the central nervous system (CNS) or in any other organ, immune and/or inflammation suppression would be beneficial, Parkinson's disease, complication and/or side effects from treatment of Parkinson's disease, AIDS-related dementia complex HIV-related encephalopathy, Devic's disease, Sydenham chorea, Alzheimer's disease and other degenerative diseases, conditions or disorders of the CNS, inflammatory components of strokes, post-polio syndrome, immune and inflammatory components of psychiatric disorders, myelitis, encephalitis, subacute sclerosing pan-encephalitis, encephalomyelitis, acute neuropathy, subacute neuropathy, chronic neuropathy, Guillaim-Barre syndrome, Sydenham chora, pseudo-tumour cerebri, Down's Syndrome, Huntington's disease, amyotrophic lateral sclerosis, inflammatory components of CNS compression or CNS trauma or infections of the CNS, inflammatory components of muscular atrophies and dystrophies, and immune and inflammatory related diseases, conditions or disorders of the central and peripheral nervous systems, post-traumatic inflammation, septic shock, infectious diseases, inflammatory complications or side effects of surgery or organ, inflammatory and/or immune complications and side effects of gene therapy, e.g. due to infection with a viral carrier, or inflammation associated with AIDS, to suppress or inhibit a humoral and/or cellular immune response, to treat or ameliorate monocyte or leukocyte proliferative diseases, e.g. leukaemia, by reducing the amount of monocytes or lymphocytes, for the prevention and/or treatment of graft rejection in cases of transplantation of natural or artificial cells, tissue and organs such as cornea, bone marrow, organs, lenses, pacemakers, natural or artificial skin tissue.

57. The use of a modulator of Notch signalling to treat a disease associated with excessive IL-4 production.

58. The method or use as described in any one of the preceding paragraphs wherein the modulator of Notch signalling comprises a protein or polypeptide comprising a Notch ligand DSL domain or a polynucleotide sequence coding for such a protein or polypeptide.

59. The method or use as described in any one of the preceding paragraphs wherein the modulator of Notch signalling comprises a protein or polypeptide comprising a Notch ligand DSL domain and at least one EGF-like domain or a polynucleotide sequence coding for such a protein or polypeptide.

60. The method or use as described in any one of the preceding paragraphs wherein DSL or EGF domains are from Delta or Jagged.

61. The method or use as described in any one of the preceding paragraphs wherein the modulator of the Notch signalling pathway comprises a fusion protein comprising a segment of a Notch ligand extracellular domain and an immunoglobulin F_{c} segment or a polynucleotide coding for such a fusion protein.

62. The method or use as described in any one of paragraphs 1 to 60 wherein modulator of the Notch signalling pathway comprises a Notch intracellular domain (Notch IC) or a polynucleotide sequence which codes for a Notch intracellular domain.

63. A method for detecting, measuring or monitoring Notch signalling comprising determining the amount of a Th1- or Th2-specific transcription factor protein, polypeptide or polynucleotide or determining the amount of a polynucleotide coding for such a protein or polypeptide.

64. A method as described in paragraph 63 wherein the amount of the Th1- or Th2-specific transcription factor protein, polypeptide or polynucleotide in a biological sample taken from a subject is determined.

65. A method as described in paragraph 64 wherein the sample comprises blood, serum, urine, lymphatic fluid, or tissue.

66. A method as described in paragraph 64 or 65 wherein the sample comprises an immune cell.

67. A method as described in paragraph 64 or paragraph 65 wherein the sample comprises a cancer cell.

68. A method as described in paragraph 64 or paragraph 65 wherein the sample comprises a stem cell.

69. A method as described in paragraph 66 wherein the sample comprises peripheral T-cells.

70. A method as described in any of paragraphs 63 to 69 comprising the steps of:

i) obtaining a biological sample from a subject; and

ii) contacting the biological sample with a binding agent that binds to a Th1- or Th2-specific transcription factor, polypeptide or polynucleotide.

71. A method as described in paragraph 70 comprising the steps of:

i) obtaining a biological sample from a subject;

ii) contacting the biological sample with a binding agent that binds to a Th1- or Th2-specific transcription factor protein, polypeptide or polynucleotide; and

iii) detecting in the sample an amount of Th1- or Th2-specific transcription factor protein, polypeptide or polynucleotide that binds to the binding agent.

72. A method as described in paragraph 71, comprising the steps of:

i) obtaining a biological sample from the patient;

ii) contacting the biological sample with a binding agent that binds to a Th1- or Th2-specific transcription factor protein, polypeptide or polynucleotide;

iii) detecting in the sample an amount of Th1- or Th2-specific transcription factor protein, polypeptide or polynucleotide that binds to the binding agent; and

iv) comparing the amount of Th1- or Th2-specific transcription factor protein, polypeptide or polynucleotide to a reference value and therefrom determining the degree of Notch signalling.

73. A method as described in any one of paragraphs 70 to 72 wherein the binding agent is a protein or polypeptide

74. A method as described in paragraph 73 wherein the binding agent is an antibody or antibody fragment.

75. A method as described in paragraph 74 wherein the antibody or antibody fragment is specific for a human Th1- or Th2-specific transcription factor.

76. A method as described in paragraph 75 wherein the antibody is raised against a human Th1- or Th2-specific transcription factor.

77. A method as described in any of paragraphs 70 to 72 wherein the binding agent is a polynucleotide.

78. A method as described in paragraph 77 comprising the further step of amplifying a Th1- or Th2-specific transcription factor polynucleotide in a sample and detecting the amplified polynucleotide.

79. A method as described in paragraph 78 wherein the amplification is by PCR.

80. A method as described in paragraph 79 wherein the amplification is by real-time PCR.

81. A method of detecting, measuring or monitoring Notch signalling in an immune cell by determining the amount of a Th1- or Th2-specific transcription factor protein, polypeptide or polynucleotide or a polynucleotide coding for such a protein or polypeptide in the cell.

82. A method as described in paragraph 81 wherein the immune cell is a T-cell.

83. A method as described in paragraph 81 wherein the immune cell is a B-cell.

84. A method as described in paragraph 81 wherein the immune cell is an antigen presenting cell.

85. A method for detecting, measuring or monitoring immunological tolerance or activity comprising determining the amount of a Th1- or Th2-specific transcription factor protein, polypeptide or polynucleotide or a polynucleotide coding for such a protein or polypeptide.

86. A method for detecting, measuring or monitoring T-cell activity comprising determining the amount of a Th1- or Th2-specific transcription factor protein, polypeptide or polynucleotide or a polynucleotide coding for such a protein or polypeptide.

87. A method for detecting, measuring or monitoring the reactivity of a T-cell to an antigen comprising determining the amount of a Th1- or Th2-specific transcription factor protein, polypeptide or polynucleotide or a polynucleotide coding for such a protein or polypeptide.

88. A method according to any of paragraphs 62 to 88 further comprising a step of comparing the amount of a Th1- or Th2-specific transcription factor protein, polypeptide or polynucleotide or a polynucleotide coding for such a protein or polypeptide with a reference amount.

89. A method according to any of paragraphs 62 to 88 wherein the amount of Th1- or Th2-specific transcription factor protein, polypeptide or polynucleotide or a polynucleotide coding for such a protein or polypeptide is detected using a nucleic acid assay.

90. A method according to any of paragraphs 62 to 88 wherein the amount of Th1- or Th2-specific transcription factor protein, polypeptide or polynucleotide or a polynucleotide coding for such a protein or polypeptide is detected using a protein assay.

91. A diagnostic kit comprising a binding agent that binds to a Th1- or Th2-specific transcription factor protein, polypeptide or polynucleotide for detecting, measuring or monitoring Notch signalling.

### REFERENCES (INCORPORATED HEREIN BY REFERENCE)

Artavanis-Tsakonas et al. (1995) Science 268:225-232.
Artavanis-Tsakonas et al. (1999) Science 284:770-776.
Atschul et al. (1990) J. Mol. Biol. 403-410.
Brucker et al. (2000) Nature 406:411-415.
Camilli et al. (1994) Proc Natl Acad Sci USA 91:2634-2638.
Caux et al. (1992) Nature 360:258-261.
Chee et al. (1996) Science 274:601-614.
Coffin et al. (1998) Gene Therapy 5:718-722.
Devereux et al. (1984) Nucleic Acid Research 12:87.
Dkuz et al. (1997) Cell 90: 271-280.
Hicks et al. (2000) Nat. Cell. Biol. 2:515-520.
Hoyne et al. (2000) Immunology 100:281-288.
Hoyne et al. (2001)—reference in press.
Inaba et al. (1992) J. Exp. Med. 175:1157-1167.
Irvine (1999) Curr. Opin. Genet. Devel. 9:434-441.
Ju et al. (2000) Nature 405:191-195.
Kroll et al. (1993) DNA Cell Biol. 12:441-453.
Leimeister et al. (1999) Mech Dev 85(1-2):173-7.
Li et al. (1998) Immunity 8(1):43-55.
Lieber et al. (1993) Genes Dev 7(10):1949-65.
Lu et al. (1996) Proc Natl Acad Sci 93(11):5663-7.
Maddox (1983) J. Exp. Med. 15(8):121 1
Matsuno et al. (1995) Development 121(8):2633-44
Matsuno et al. (1998) Nat. Genet. 19:74-78.
McGuinness T. et al. (1996) Genomics 35(3):473-85.
Moloney et al. (2000) Nature 406:369-375.
Munro and Freeman (2000) Curr. Biol. 10:813-820.
Ordentlich et al. (1998) Mol. Cell. Biol. 18:2230-2239.
Osborne and Miele (1999) Immunity 11:653-663.
Panin et al. (1997) Nature 387:908-912.
Sauber et al. (1995) Virol. 213:439-449
Schroeter et al. (1998) Nature 393(6683):382-6.
Struhl et al. (1998) Cell 93(4):649-60.
Takebayashi et al. (1994) J Biol Chem 269(7):150-6.
Tamura et al. (1995) Curr. Biol. 5:1416-1423.
Weinmaster (2000) Curr. Opin. Genet. Dev. 10:363-369.

Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in chemistry, biology or related fields are intended to be within the scope of the following claims.

We claim:

1. A method for modifying IL-4 expression in a cell comprising contacting the cell with a modulator of Notch signalling.

2. The method of claim 1, wherein the modulator is an activator of Notch signalling and wherein IL-4 expression is increased.

3. The method of claim 2, wherein the modulator is a Notch receptor agonist.

4. The method of claim 1, wherein the modulator is an inhibitor of Notch signalling and wherein IL-4 expression is decreased.

5. The method of claim 4, wherein the modulator is a Notch receptor antagonist.

6. The method of claim 1, wherein the modulator comprises a protein or polypeptide comprising a Notch ligand DSL domain or a polynucleotide sequence encoding the protein or polypeptide.

7. The method of claim 6, wherein the modulator comprises a protein or polypeptide further comprising at least one EGF-like domain or a polynucleotide sequence encoding the protein or polypeptide.

8. The method of claim 7, wherein DSL or EGF domains are from Delta or Jagged.

9. The method of claim 1, wherein the modulator comprises a fusion protein comprising a segment of a Notch ligand extracellular domain and an immunoglobulin Fc segment or a polynucleotide encoding the fusion protein.

10. The method of claim 1, wherein the modulator comprises a Notch intracellular domain (Notch IC) or a polynucleotide sequence encoding a Notch IC.

11. The method of claim 1, wherein the cell is a leukocyte, macrophage, fibroblast or epithelial cell.

12. The method of claim 6, wherein the leukocyte is a lymphocyte.

13. A method for generating, in a cell, an immune modulatory cytokine profile with increased IL-10 expression and increased IL-4 expression, the method comprising contacting the cell with an activator of Notch signalling.

14. A method for generating, in a cell, an immune modulatory cytokine profile with increased IL-4 expression and reduced IL-5, IL-13 and TNFα expression, the method comprising contacting the cell with a modulator of Notch signalling.

15. The method of claim 14, wherein IL-2 and IFNγ expression are decreased.

16. The method of claim 14, IL-10 expression is increased.

17. A method for increasing a TH2 immune response and/or decreasing a TH1 immune response in a cell, the method comprising contacting the cell with a modulator of Notch signalling.

18. A method for treating inflammation or an inflammatory or autoimmune condition in a subject comprising administering to the subject a modulator of Notch signalling to increase IL-4 expression and reduce a TH1 immune response.

19. The method of claim 18, wherein the modulator of Notch signalling is administered to a cell ex vivo, and the cell is subsequently administered to the subject.

20. The method of claim 18, wherein the inflammation or inflammatory or autoimmune condition is associated with excessive IL-4 production.
